(19) United States
(12) Patent Application Publication
(10) Pub. No.: US 2024/0150313 A1
(43) Pub. Date: May 9, 2024

(54) TRI-SUBSTITUTED INDOLE BINDING FUNCTION 3 (BF3) COMPOUNDS AND METHODS FOR THEIR USE

(71) Applicants: The University of British Columbia, Vancouver (CA); Simon Fraser University, Burnaby (CA)

(72) Inventors: Christophe ANDRE, Burnaby (CA); Gang CHEN, Burnaby (CA); Artem TCHERKASOV, Vancouver (CA); Nathan LACK, Istanbul (TR); Eric J. LeBLANC, Vancouver (CA); Sankar MOHAN, Burnaby (CA); Paul S. RENNIE, Richmond (CA); Fuqiang BAN, Vancouver (CA); Robert YOUNG, Burnaby (CA)

(21) Appl. No.: 18/554,759

(22) PCT Filed: Apr. 12, 2022
(86) PCT No.: PCT/IB2022/053446

§ 371 Date: Oct. 10, 2023

Related U.S. Application Data
(60) Provisional application No. 63/173,890, filed on Apr. 12, 2021.

(51) Int. Cl. C07D 401/04; A61P 35/00; C07D 413/14; C07D 417/04; C07D 417/14; C07F 9/6558

(52) U.S. Cl. CPC C07D 401/04; A61P 35/00; C07D 413/14; C07D 417/04; C07D 417/14; C07F 9/65583

(57) ABSTRACT

Provided herein are uses of the compounds for the treatment of various indications, including prostate cancer and Kennedy's disease.

### RELATED APPLICATIONS

This application claims priority to U.S. Provisional Application No. 63/173,890 filed on Apr. 12, 2022, the entire content of which is hereby incorporated by reference in its entirety.

### TECHNICAL FIELD

This present disclosure relates to therapeutics, their uses and methods for the treatment of various indications, including various androgen receptor mediated diseases. In particular, the present disclosure relates to therapies and methods of treatment for cancers such as prostate cancer, as well as Kennedy's disease.

### BACKGROUND

Prostate cancer is the second leading cause of male cancer-related death in Western countries (Damber, J. E. and Aus, G. Lancet (2008) 371:1710-1721). Numerous studies have shown that the androgen receptor (AR) is central not only to the development of prostate cancer, but also the progression of the disease to the castration resistance state (Taplin, M. E. et al. J. Clin. Oncol. (2003) 21:2673-8; and Tilley, W. D. et al. Cancer Res. (1994) 54:4096-4102).

Kennedy's disease or Spinal Bulbar Muscular Atrophy (SBMA) is an x-liked recessive motor neuron disease resulting from disruptions in the transmission of nerve cell signals in the brain stem and spinal cord. The nerve cells in a Kennedy's patient gradually become increasingly dysfunctional and eventually die, leaving the muscles unable to contract, resulting in atrophy of the muscles throughout the body, but most noticeably in the extremities, face and throat. The motor neuron disruptions are more noticeable relative to other cells because of the higher number of the androgen receptors residing in nerve cells. The binding of testosterone to the androgen receptor is thought to cause the disease. At present there is no treatment for Kennedy's disease.

There remains a need for effective treatments for cancer, such as prostate cancer, as well as Kennedy's disease.

### SUMMARY

Provided herein are compounds that modulate androgen receptor (AR) activity and therefore treat prostate cancer and Spinal Bulbar Muscular Atrophy (SBMA, or Kennedy's disease).

In an aspect, provided herein is a compound of Formula I:

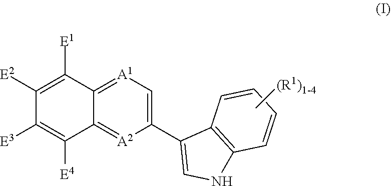

or a pharmaceutically acceptable salt thereof.

In another aspect, provided herein is a compound of Formula II:

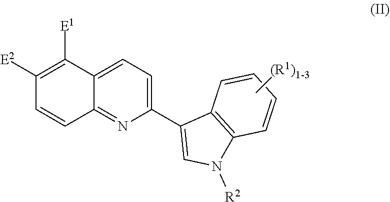

or a pharmaceutically acceptable salt thereof.

In yet another aspect, provided herein is a compound of Formula III:

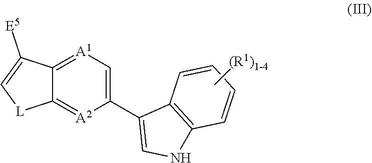

or a pharmaceutically acceptable salt thereof.

In still another aspect, provided herein is a pharmaceutical composition comprising a compound of any of Formulae I-III, or a pharmaceutically acceptable salt thereof, and a pharmaceutically acceptable carrier.

In an aspect, provided herein is a method of treating Spinal Bulbar Muscular Atrophy (SBMA) in a subject in need thereof comprising administering to the subject a therapeutically effective amount of a compound described herein.

In another aspect, provided herein is a method of treating prostate cancer in a subject in need thereof comprising administering to the subject a therapeutically effective amount of a compound described herein.

### BRIEF DESCRIPTION OF THE DRAWINGS

FIG. 1 shows the effect of compound 13789 and 13789 prodrug on AR transcriptional activity. A) Dose-response curve illustrating the inhibiting effect of compound 13789 on the AR transcriptional activity. B) compound 13789 inhibits AR-mediated PSA expression in LNCaP cells (in vitro).

FIG. 2 shows the effect of compound 13789 on cell proliferation in LNCaP, MR49F and PC3 (AR negative) cells.

FIG. 3 shows that treatment with 13789 prodrug inhibits PSA production in a LNCaP xenograft castration-resistant mouse model. Error bars indicate standard error of the mean. T-Test p-values for Vehicle vs. 13789 Prodrug are 0.016 at 3 weeks and 0.046 at 5 weeks. MDV=Enzalutamide.

FIG. 4 shows that treatment with 13789 prodrug reduces tumor volume in a LNCaP xenograft castration-resistant mouse model. Plots show tumour volumes from mice from each treatment group for up to 5 weeks of treatment. Error bars indicate standard error of the mean value. T-Test p-values for Vehicle vs. 13789 Prodrug are 0.024 at 4 weeks and 0.023 at 5 weeks. MDV=Enzalutamide.

FIG. 5 shows a representative chromatogram of a mixture of 13566, 13789, and 13822 (ca. 0.3 μg/mL in 50% ACN).

FIG. 6 shows a calibration curve for 13566, 13789 and 13822.

FIG. 7 shows the relative errors from mean for repeated injections, whereby QC blocks consisting two blanks and one injection of a 3-component mixture at 0.3 μg/mL were inserted throughout the calibration curve and sample analysis sequence, and the results of repeated injections were plotted (as percent differences from mean values—standard error of 2.2%, 1.5%, and 1.4% for 13566, 13789, and 13822, respectively).

FIG. 8 shows a summary of 13566 and 13789 blood (plasma) and brain levels for each of 13789 and 13566.

FIG. 9 shows a PK profile of 13789 (dosed as its prodrug 13822).

FIG. 10 shows representative serum and brain chromatograms for 13566 samples.

FIG. 11 shows representative serum and brain chromatograms for 13789 samples.

FIG. 12 shows the docked pose of the quinoline 5-amide-derived analog of 13789 demonstrating a favorable hydrogen-bonding interaction between amide-NH and the carbonyl of Asn833.

FIG. 13 shows in vitro AR antagonism of 13789 A) Dose-response curve illustrating the inhibiting effect of 13789 (IC₅₀=0.19 μM) and enzalutamide (IC₅₀=0.075 μM) on the AR transcriptional activity in LNCaP-eGFP cells. B) The inhibition effect of 13789 (IC₅₀=0.45 μM) on AR-mediated PSA expression in LNCaP cells. C) The effect of 13789 on cell proliferation in LNCaP and PC3 (AR negative) cells. 13789 showed IC₅₀ values of 0.32 μM and 0.38 μM respectively. All data are presented as mean±SEM.

FIG. 14 shows the characterization of 13789 AR antagonism. A) GSEA analysis of differentially expressed genes in LNCaP treated with 10 nM DHT and VPC-13789 or enzalutamide. B) Heatmap of genes from the GSEA-MySigDB “Hallmark androgen response”. C) ChIP-PCR of cells treated with 13789 or enzalutamide at KLK3 (AREIII) and FKBP5 AR binding sites. D) Nuclear translocation of exogenous (AR-eGFP in 293T) or endogenous (LNCaP) AR following treatment with DHT.

FIG. 15 shows the identification of co-regulatory proteins disrupted by 13789. A) Using RIME, 13789 was found to disrupt 34 AR protein-protein interactions on chromatin. B) ZMIZ1-AR interactions were docked and scored with PRISM and PRODIGY. C) 13789 is predicted to interact with critical A residues involved in ZMIZ1 protein-protein interactions.

FIG. 16 shows predicted metabolites of 13566 caused by CYP3A4, CYP2C9, CYP1A2, CYP2D6 enzymes.

### DETAILED DESCRIPTION

Androgens are known to mediate their effects through the androgen receptor (AR). Androgens play a role in a wide range of developmental and physiological responses, for example, male sexual differentiation, maintenance of spermatogenesis, and male gonadotropin regulation (R. K. Ross, G. A. Coetzee, C. L. Pearce, J. K. Reichardt, P. Bretsky, L. N. Kolonel, B. E. Henderson, E. Lander, D. Altshuler & G. Daley, Eur Urol 35, 355-361 (1999); A. A. Thomson, Reproduction 121, 187-195 (2001); N. Tanji, K. Aoki & M. Yokoyama, Arch Androl 47, 1-7 (2001)). Also, androgens are associated with the development of prostate carcinogenesis. Induction of prostatic carcinogenesis in rodent models has been associated with androgens (R. L. Noble, Cancer Res 37, 1929-1933 (1977); R. L. Noble, Oncology 34, 138-141 (1977)) and men receiving androgens in the form of anabolic steroids are reported to have a higher incidence of prostate cancer (J. T. Roberts & D. M. Essenhigh, Lancet 2, 742 (1986); J. A. Jackson, J. Waxman & A. M. Spiekerman, Arch Intern Med 149, 2365-2366 (1989); P. D. Guinan, W. Sadoughi, H. Alsheik, R. J. Ablin, D. Alrenga & I. M. Bush, Am J Surg 131, 599-600 (1976)). Furthermore, prostate cancer does not develop if humans or dogs are castrated before puberty (J. D. Wilson & C. Roehrborn, J Clin Endocrinol Metab 84, 4324-4331 (1999); G. Wilding, Cancer Surv 14, 113-130 (1992)). Castration of adult males causes involution of the prostate and apoptosis of prostatic epithelium (E. M. Bruckheimer & N. Kyprianou, Cell Tissue Res 301, 153-162 (2000); J. T. Isaacs, Prostate 5, 545-557 (1984)). This dependency on androgens provides the underlying rationale for treating prostate cancer with chemical or surgical castration (i.e. androgen ablation).

Effective inhibition of human AR remains one of the most effective therapeutic approaches to the treatment of advanced, metastatic prostate cancer. The AR possesses a modular organization characteristic of all nuclear receptors. It is comprised of an N-terminal domain, a central DNA binding domain, a short hinge region, and C-terminal domain that contains a hormone ligand binding pocket and the Activation Function-2 (AF2) site (Gao, W. Q. et al. Chem. Rev. (2005) 105:3352-3370). The latter represents a hydrophobic groove on the AR surface which is flanked with regions of positive and negative charges—“charge clamps” that are significant for binding AR activation factors (Zhou, X. E. et al. J. Biol. Chem. (2010) 285:9161-9171). Recent studies have identified a novel site on the AR called Binding Function 3 (BF3) that is involved into AR transcriptional activity.

Notably, the current anti-androgens such as bicalutamide, flutamide, nilutamide and MDV3100, all target this particular process. However, instead of affecting the AR-cofactor interaction directly, these anti-androgens act indirectly, by binding to the AR ligand binding site. Thus, by preventing androgens from binding they also prevent conformational changes of the receptor that are necessary for co-activator interactions. While treatment with these AR inhibitors can initially suppress the prostate cancer growth, long term hormone therapy becomes progressively less effective (Taplin, M. E. et al. J. Clin. Oncol. (2003) 21:2673-8; and Tilley, W. D. et al. Cancer Res. (1994) 54:4096-4102). Factors that make the AR less sensitive to conventional anti-androgens include resistance mutations at the ligand binding site that can even lead AR antagonists to act as agonists further contributing to cancer progression (Chen, Y. et al. Lancet Oncol. (2009) 10:981-991).

Androgens also play a role in female cancers. One example is ovarian cancer where elevated levels of androgens are associated with an increased risk of developing ovarian cancer (K. J. Helzlsouer, et al., JAMA 274, 1926-1930 (1995); R. J. Edmondson, et al, Br J Cancer 86, 879-885 (2002)). The AR has been detected in a majority of ovarian cancers (H. A. Risch, J Natl Cancer Inst 90, 1774-1786 (1998); B. R. Rao & B. J. Slotman, Endocr Rev 12, 14-26 (1991); G. M. Clinton & W. Hua, Crit Rev Oncol Hematol 25, 1-9 (1997)), whereas estrogen receptor-alpha (ERa) and the progesterone receptor are detected in less than 50% of ovarian tumors.

The BF3 site is an attractive target for direct inhibition of the androgen receptor (AR) co-activation. The inhibition of AR co-activation can aid in the treatment of prostate cancer and Kennedy's Disease. As such, the compounds provided herein are capable of inhibiting AR co-activation and therefore can be useful in the treatment of diseases and disorders associated with the AR.

### Definitions

As used herein, the term “alkyl,” by itself or as part of another substituent means, unless otherwise stated, a straight or branched chain hydrocarbon having the number of carbon atoms designated (i.e., C₁₋₆ alkyl means an alkyl having one to six carbon atoms) and includes straight and branched chains. In an embodiment, C₁₋₆ alkyl groups are provided herein. Examples include methyl, ethyl, propyl, isopropyl, butyl, isobutyl, tert-butyl, pentyl, neopentyl, and hexyl. Other examples of C₁₋₆ alkyl include ethyl, methyl, isopropyl, isobutyl, n-pentyl, and n-hexyl.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” means a non-aromatic carbocyclic system that is partially or fully saturated having 1, 2 or 3 rings wherein such rings may be fused. The term “fused” means that a second ring is present (i.e., attached or formed) by having two adjacent atoms in common (i.e., shared) with the first ring. Cycloalkyl also includes bicyclic structures that may be bridged or spirocyclic in nature with each individual ring within the bicycle varying from 3-8 atoms. The term “cycloalkyl” includes, but is not limited to, cyclopropyl, cyclobutyl, cyclopentyl, cyclohexyl, bicyclo[3.1.0]hexyl, spiro[3.3]heptanyl, and bicyclo[1.1.1]pentyl. In an embodiment, C₃₋₆ cycloalkyl groups are provided herein.

The term “haloalkyl” as used herein refers to an alkyl group in which one or more of the hydrogen atoms has been replaced by a halogen atom. The term “Cₙ₋ₘ haloalkyl” refers to a Cₙ₋ₘ alkyl group having n to m carbon atoms and from at least one up to {2(n to m)+1} halogen atoms, which may either be the same or different. In some embodiments, the halogen atoms are fluoro atoms. In some embodiments, the haloalkyl group has 1 to 6 or 1 to 4 carbon atoms. Example haloalkyl groups include CF₃, C₂F₅, CHF₂, CH₂F, CCl₃, CHCl₂, C₂Cl₅ and the like. In some embodiments, the haloalkyl group is a fluoroalkyl group.

The term “heteroaryl” or “heteroaromatic,” employed alone or in combination with other terms, refers to a monocyclic or polycyclic aromatic heterocycle having at least one heteroatom ring member selected from sulfur, oxygen and nitrogen. In some embodiments, the heteroaryl ring has 1, 2, 3 or 4 heteroatom ring members independently selected from nitrogen, sulfur and oxygen. In some embodiments, any ring-forming N in a heteroaryl moiety can be an N-oxide. In some embodiments, the heteroaryl has 5-14 ring atoms including carbon atoms and 1, 2, 3 or 4 heteroatom ring members independently selected from nitrogen, sulfur and oxygen. In some embodiments, the heteroaryl has 5-10 ring atoms including carbon atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. In other embodiments, the heteroaryl is an eight-membered, nine-membered or ten-membered fused bicyclic heteroaryl ring. Example heteroaryl groups include, but are not limited to, pyridinyl (pyridyl), pyrimidinyl, pyrazinyl, pyridazinyl, pyrrolyl, pyrazolyl, azolyl, oxazolyl, isoxazolyl, thiazolyl, imidazolyl, furanyl, thiophenyl, quinolinyl, isoquinolinyl, naphthyridinyl (including 1,2-, 1,3-, 1,4-, 1,5-, 1,6-, 1,7-, 1,8-, 2,3- and 2,6-naphthyridine), indolyl, isoindolyl, benzothiophenyl, benzofuranyl, benzisoxazolyl, imidazo[1,2-b]thiazolyl, purinyl, and the like. In some embodiments, the heteroaryl group is pyridone (e.g., 2-pyridone).

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S, In another embodiment, isotopically-labeled compounds are useful in drug or substrate tissue distribution studies. In another embodiment, substitution with heavier isotopes such as deuterium affords greater metabolic stability (for example, increased in vivo half-life or reduced dosage requirements). In yet another embodiment, the compounds described herein include a ²H (i.e., deuterium) isotope.

In still another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

It will be understood by a person of skill that COOH and NR₂ may include the corresponding ions, for example carboxylate ions and ammonium ions, respectively. Alternatively, where the ions are shown, a person of skill in the art will appreciate that the counter ion may also be present.

Those skilled in the art will appreciate that the point of covalent attachment of the moiety to the compounds as described herein may be, for example, and without limitation, cleaved under specified conditions. Specified conditions may include, for example, and without limitation, in vivo enzymatic or non-enzymatic means. Cleavage of the moiety may occur, for example, and without limitation, spontaneously, or it may be catalyzed, induced by another agent, or a change in a physical parameter or environmental parameter, for example, an enzyme, light, acid, temperature or pH. The moiety may be, for example, and without limitation, a protecting group that acts to mask a functional group, a group that acts as a substrate for one or more active or passive transport mechanisms, or a group that acts to impart or enhance a property of the compound, for example, solubility, bioavailability or localization.

Pharmaceutically acceptable salts of the compounds described herein are also provided. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 17.sup.th ed., Mack Publishing Company, Easton, Pa., 1985, p. 1418 and Journal of Pharmaceutical Science, 66, 2 (1977), each of which is incorporated herein by reference in its entirety.

Compounds described herein may be in the free form or in the form of a prodrug thereof. The term prodrug as used herein refers to a compound that undergoes a chemical conversion, through a metabolic process, enzymatic conversion, or otherwise, into its active form.

In some embodiments, compounds and all different forms thereof (e.g. free forms, prodrugs, salts, polymorphs, isomeric forms) as described herein may be in the solvent addition form, for example, solvates.

In some embodiments, compounds and all different forms thereof (e.g. free forms, prodrugs, salts, solvates, isomeric forms) as described herein may include crystalline and amorphous forms.

In some embodiments, compounds as described herein include isomers such as geometrical isomers, optical isomers based on asymmetric carbon, stereoisomers, tautomers, individual enantiomers, individual diastereomers, racemates, diastereomeric mixtures and combinations thereof, and are not limited by the description of the formula illustrated for the sake of convenience.

In some embodiments, pharmaceutical compositions as described herein may comprise a salt or prodrug of such a compound, preferably a pharmaceutically or physiologically acceptable salt or prodrug. Pharmaceutical preparations will typically comprise one or more carriers, excipients or diluents acceptable for the mode of administration of the preparation, be it by injection, inhalation, topical administration, lavage, or other modes suitable for the selected treatment. Suitable carriers, excipients or diluents (used interchangeably herein) are those known in the art for use in such modes of administration.

Suitable pharmaceutical compositions may be formulated by means known in the art and their mode of administration and dose determined by the skilled practitioner. For parenteral administration, a compound may be dissolved in sterile water or saline or a pharmaceutically acceptable vehicle used for administration of non-water soluble compounds such as those used for vitamin K. For enteral administration, the compound may be administered in a tablet, capsule or dissolved in liquid form. The tablet or capsule may be enteric coated, or in a formulation for sustained release. Many suitable formulations are known, including, polymeric or protein microparticles encapsulating a compound to be released, ointments, pastes, gels, hydrogels, or solutions which can be used topically or locally to administer a compound. A sustained release patch or implant may be employed to provide release over a prolonged period of time. Many techniques known to one of skill in the art are described in Remington: the Science & Practice of Pharmacy by Alfonso Gennaro, 20^{th} ed., Lippencott Williams & Wilkins, (2000). Formulations for parenteral administration may, for example, contain excipients, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Compounds or pharmaceutical compositions as described herein or for use as described herein may be administered by means of a medical device or appliance such as an implant, graft, prosthesis, stent, etc. Also, implants may be devised which are intended to contain and release such compounds or compositions. An example would be an implant made of a polymeric material adapted to release the compound over a period of time.

An “effective amount” of a pharmaceutical composition as described herein includes a therapeutically effective amount or a prophylactically effective amount. A “therapeutically effective amount” refers to an amount effective, at dosages and for periods of time necessary, to achieve the desired therapeutic result, such as reduced tumor size, increased life span or increased life expectancy. A therapeutically effective amount of a compound may vary according to factors such as the disease state, age, sex, and weight of the subject, and the ability of the compound to elicit a desired response in the subject. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as smaller tumors, increased life span, increased life expectancy or prevention of the progression of prostate cancer to an androgen-independent form. Typically, a prophylactic dose is used in subjects prior to or at an earlier stage of disease, so that a prophylactically effective amount may be less than a therapeutically effective amount.

As used herein, the term “treating” or “treatment” refers to one or more of (1) preventing the disease; for example, preventing a disease, condition or disorder in an individual who may be predisposed to the disease, condition or disorder but does not yet experience or display the pathology or symptomatology of the disease; (2) inhibiting the disease; for example, inhibiting a disease, condition or disorder in an individual who is experiencing or displaying the pathology or symptomatology of the disease, condition or disorder (i.e., arresting further development of the pathology and/or symptomatology); and (3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease. In some embodiments, the term “treating” or “treatment” refers to inhibiting or ameliorating the disease.

It is to be noted that dosage values may vary with the severity of the condition to be alleviated. For any particular subject, specific dosage regimens may be adjusted over time according to the individual need and the professional judgment of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the subject. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In some embodiments, compounds and all different forms thereof as described herein may be used, for example, and without limitation, in combination with other treatment methods for at least one indication selected from the group consisting of: prostate cancer, breast cancer, ovarian cancer, endometrial cancer, hair loss, acne, hirsutism, ovarian cysts, polycystic ovary disease, precocious puberty and age-related macular degeneration. For example, compounds and all their different forms as described herein may be used as neoadjuvant (prior), adjunctive (during), and/or adjuvant (after) therapy with surgery, radiation (brachytherapy or external beam), or other therapies (eg. HIFU).

In general, compounds as described herein should be used without causing substantial toxicity. Toxicity of the compounds as described herein can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be appropriate to administer substantial excesses of the compositions. Some compounds as described herein may be toxic at some concentrations. Titration studies may be used to determine toxic and non-toxic concentrations. Toxicity may be evaluated by examining a particular compound's or composition's specificity across cell lines using PC3 cells as a negative control that do not express AR. Animal studies may be used to provide an indication if the compound has any effects on other tissues. Systemic therapy that targets the AR will not likely cause major problems to other tissues since anti-androgens and androgen insensitivity syndrome are not fatal.

Compounds as described herein may be administered to a subject. As used herein, a “subject” may be a human, non-human primate, rat, mouse, cow, horse, pig, sheep, goat, dog, cat, etc. The subject may be suspected of having or at risk for having a cancer, such as prostate cancer, breast cancer, ovarian cancer or endometrial cancer, or suspected of having or at risk for having acne, hirsutism, alopecia, benign prostatic hyperplasia, ovarian cysts, polycystic ovary disease, precocious puberty, or age-related macular degeneration. Diagnostic methods for various cancers, such as prostate cancer, breast cancer, ovarian cancer or endometrial cancer, and diagnostic methods for acne, hirsutism, alopecia, benign prostatic hyperplasia, ovarian cysts, polycystic ovary disease, precocious puberty, or age-related macular degeneration and the clinical delineation of cancer, such as prostate cancer, breast cancer, ovarian cancer or endometrial cancer, diagnoses and the clinical delineation of acne, hirsutism, alopecia, benign prostatic hyperplasia, ovarian cysts, polycystic ovary disease, precocious puberty, or age-related macular degeneration are known to those of ordinary skill in the art.

Definitions used include ligand-dependent activation of the androgen receptor (AR) by androgens such as dihydrotestosterone (DHT) or the synthetic androgen (R1881) used for research purposes. Ligand-independent activation of the AR refers to transactivation of the AR in the absence of androgen (ligand) by, for example, stimulation of the cAMP-dependent protein kinase (PKA) pathway with forskolin (FSK).

### Compounds

In an aspect, provided herein is a compound of Formula I:

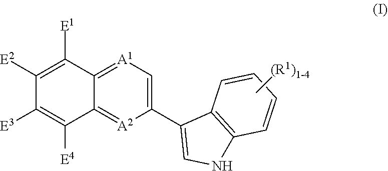

or a pharmaceutically acceptable salt thereof;
   wherein
each R¹ is independently selected from the group consisting of halo, C₁₋₆ alkyl, C₁₋₆ haloalkyl, and OC₁₋₆ alkyl;
A¹ is selected from C—OH, C(OCH₃), N, CH, CF, and CCF₃;
A² is selected from N and CH;
E¹ is selected from C(O)NH(C₁₋₆ alkyl), C(O)N(C₁₋₆ alkyl)₂, C(O)NH(C₃₋₆ cycloalkyl), halo, OH, CO₂H, and 5-membered heteroaryl, wherein alkyl and heteroaryl are each optionally substituted with 1, 2, or 3 substituents selected from halo, hydroxy, C₁₋₆ alkyl, C₁₋₆ haloalkyl, C₁₋₆ alkenyl, NO₂, CN, and C₃₋₆ cycloalkyl; and
E², E³, and E⁴ are each independently selected from H, halo, CN, NO₂, C₁₋₆ alkyl, C₂₋₆ alkenyl, OC₁₋₆ alkyl, and C₁₋₆ haloalkyl.

In an embodiment, the compound of Formula I is a compound of Formula Ia:

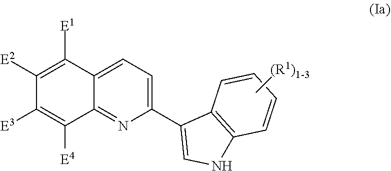

or a pharmaceutically acceptable salt thereof.

In another embodiment of Formulae I and Ia, each R¹ is independently halo or C₁₋₆ alkyl.

In yet another embodiment, the compound of Formula I is a compound of Formula Ib:

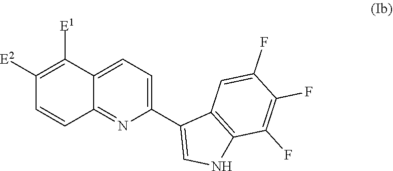

or a pharmaceutically acceptable salt thereof.

In an embodiment of Formulae I, Ia, and Ib, E² is H or halo.

In another embodiment, the compound of Formula I is

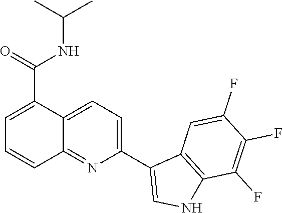 [SMILES: CC(C)NC(=O)c1cccc2nc(-c3c[nH]c4c(F)c(F)c(F)cc34)ccc12]

or a pharmaceutically acceptable salt thereof.

In another aspect, provided herein is a compound of Formula II:

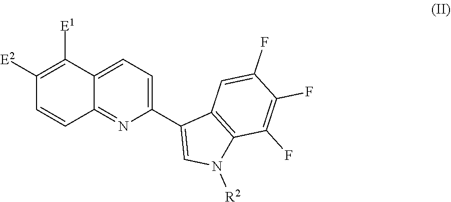

or a pharmaceutically acceptable salt thereof;
   wherein
E¹ is selected from C(O)NH(C₁₋₆ alkyl), C(O)N(C₁₋₆ alkyl)₂, C(O)NH(C₃₋₆ cycloalkyl), halo, OH, CO₂H, and 5-membered heteroaryl, wherein alkyl and heteroaryl are each optionally substituted with 1, 2, or 3 substituents selected from halo, hydroxy, C₁₋₆ alkyl, C₁₋₆ haloalkyl, C₁₋₆ alkenyl, NO₂, CN, and C₃₋₆ cycloalkyl; and
E² is selected from H, halo, CN, NO₂, C₁₋₆ alkyl, C₂₋₆ alkenyl, OC₁₋₆ alkyl, and C₁₋₆ haloalkyl; and
R² is C₁₋₆ alkyl optionally substituted with OPO₃H₂.

In an embodiment of Formula II, E¹ is C(O)NH(C₁₋₆ alkyl).

In another embodiment of Formula II, E² is H or halo. In yet another embodiment, E² is H. In still another embodiment, E² is halo.

In an embodiment, the compound of Formula II is

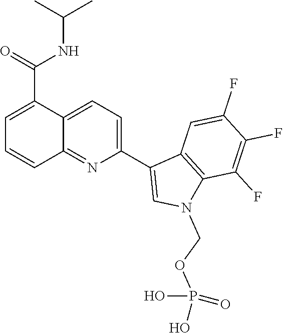 [SMILES: CC(C)NC(=O)c1cccc2nc(-c3cn(COP(=O)(O)O)c4c(F)c(F)c(F)cc34)ccc12]

or a pharmaceutically acceptable salt thereof.

In yet another aspect, provided herein is a compound of Formula III:

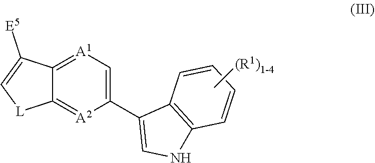

or a pharmaceutically acceptable salt thereof;
   wherein
each R¹ is independently selected from the group consisting of halo, C₁₋₆ alkyl, C₁₋₆ haloalkyl, and OC₁₋₆ alkyl;
A¹ is selected from C—OH, C(OCH₃), N, CH, CF, and CCF₃;
A² is selected from N and CH;
L is selected from NH, O, and S; and
E⁵ is C(O)NH(C₁₋₆ alkyl).

In an embodiment, the compound of Formula III is a compound of Formula IIIa:

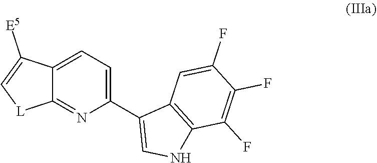

or a pharmaceutically acceptable salt thereof.

In still another aspect, provided herein is a pharmaceutical composition comprising a compound of any one of the compounds described herein, or a pharmaceutically acceptable salt thereof, and a pharmaceutically acceptable carrier.

Some compounds and compositions as described herein may interfere with a mechanism specific to ligand-dependent activation (e.g., accessibility of the ligand binding domain (LBD) to androgen) or to ligand-independent activation of the AR.

### Methods of Treatment

In an aspect, provided herein is a method of treating Spinal Bulbar Muscular Atrophy (SBMA) in a subject in need thereof comprising administering to the subject a therapeutically effective amount of a compound described herein or a pharmaceutically acceptable salt thereof.

In another aspect, provided herein is a method of treating prostate cancer in a subject in need thereof comprising administering to the subject a therapeutically effective amount of a compound disclosed herein or a pharmaceutically acceptable salt thereof.

In an embodiment, the prostate cancer is castration-resistant prostate cancer (CRPC).

In yet another aspect, provided herein is a method of modulating androgen receptor (AR) activity in a subject in need thereof comprising administering to the subject a therapeutically effective amount of a compound disclosed herein, or a pharmaceutically acceptable salt thereof.

In still another aspect, provided herein is a method of treating cancer in a subject in need thereof comprising administering to the subject a therapeutically effective amount of a compound disclosed herein or a pharmaceutically acceptable salt thereof.

In an embodiment, the cancer is selected from the group consisting of prostate cancer, breast cancer, ovarian cancer, and endometrial cancer.

In an aspect, provided herein is a method of treating a disease or disorder associated with the androgen receptor (AR) in a subject in need thereof comprising administering to the subject a therapeutically effective amount of a compound disclosed herein or a pharmaceutically acceptable salt thereof.

In an embodiment, the disease or disorder associated with the androgen receptor (AR) is selected from the group consisting of hair loss, acne, hirsutism, ovarian cysts, polycystic ovary disease, precocious puberty, and age-related macular degeneration.

In some embodiments the compound of Formula I may be used in the preparation of a medicament or a composition for systemic treatment of an indication described herein. In some embodiments, methods of systemically treating any of the indications described herein are also provided.

Various alternative embodiments and examples of the present disclosure are described herein. These embodiments and examples are illustrative and should not be construed as limiting in scope.

### EXAMPLES

### General Synthesis and Characterization of Compounds

General Methods: ¹H and ¹³C NMR spectra were recorded at 600 or 500 MHz and 150 or 125 MHz, respectively. All assignments were confirmed with the aid of two-dimensional ¹H-¹H (COSY) and/or ¹H-¹³C (HSQC) and ¹H-¹³C (HMBC) experiments and attached proton test (APT) using standard pulse programs. Processing of the spectra was performed with MestReNova software. Analytical thin-layer chromatography (TLC) was performed on aluminum plates precoated with silica gel 60E-254 as the adsorbent. The developed plates were air-dried, exposed to UV light and/or dipped in KMnO₄ solution and heated. Column chromatography was performed using Biotage automated purification system and Silicycle columns. High resolution mass spectra were obtained by the electrospray ionization method, using an Agilent 6210 TOF LC/MS high resolution mass spectrometer.

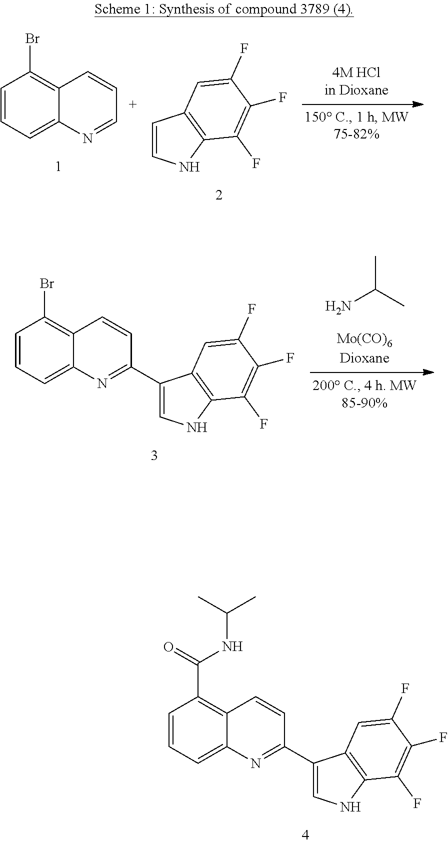

5-Bromo-2-(5,6,7-trifluoro-1H-indol-3-yl) quinoline (3): 5-Bromoquinoline 1 (2.8 g, 13.44 mmol) and trifluoroindole 2 (1.0 g, 5.84 mmol) were mixed well in a 20 mL microwave vial. Mixed solids melted spontaneously and formed a brown gum. Then, 4M HCl solution in 1,4-dioxane (2.0 mL, 8.18 mmol) was added to the vial which resulted in a reddish orange suspension. Reaction vial was sealed and irradiated in a Biotage microwave reactor at 150° C. for 1 h without stirring. Resulting reddish orange brick-like solid was suspended in MeOH (90 mL) using sonication and the slurry was transferred to a separatory funnel and diluted with EtOAc (300 mL) and washed with saturated NaHCO₃ (150 mL). The aqueous layer was extracted with EtOAc (100 mL). Combined EtOAc layers were concentrated to dryness and the crude was purified by flash chromatography (Silica gel, a gradient from 30% DCM/Hexanes to 50% DCM and then 100% DCM) to give the title compound 3 as a pale yellow solid (1.8 g, 80%).

¹H NMR (500 MHz, DMSO-d₆): δ 12.57 (s, 1H, —NH), 8.65 (br-ddd-like, J_{H-F}=11.1, 7.2 Hz, 1H, H₄), 8.62 (s, 1H, H₂), 8.40 (d, J=9.0 Hz, 1H, H_{4′}), 8.23 (d, J=9.0 Hz, 1H, H_{3′}), 8.14 (d, J=8.4 Hz, 1H, H_{6′}), 7.85 (d, J=7.5 Hz, 1H, H_{8′}), 7.66 (t, J=8.0 Hz, 1H, H_{7′}).

HRMS: calculated for C₁₇H₉BrF₃N₂ [M+H]⁺: 376.9896 and 378.9877, found: 376.9894 and 378.9875.

TLC: 0.3 R_{f} in 20% EtOAc/Hex

N-isopropyl-2-(5,6,7-trifluoro-1H-indol-3-yl)quinoline-5-carboxamide (4): A suspension of 5-bromo-2-(5,6,7-trifluoro-1H-indol-3-yl) quinoline 3 (1.65 g, 4.37 mmol) in 1,4-dioxane (12 mL) in a 20 mL microwave vial was warmed until a clear solution was obtained. To this warm solution under stirring, Mo(CO)₆ (1.2 g, 4.37 mmol) was added in one portion followed by isopropyl amine (2.2 mL, 26.22 mmol). The reaction vial was then sealed and irradiated in Biotage microwave reactor at 200° C. for 4 h (Caution: pressure builds up during reaction (15-17 bar), protective shield surrounding the reactor is highly recommended. Scale up was done in batches of 2 g scale). After this time, the reaction mixture was directly loaded on a silica gel column and purified using a gradient of 30% EtOAc/Hex to 100% EtOAc and then 20% MeOH/EtOAc. Fractions eluted were found to be slightly impure, contained small amounts of both polar and non-polar impurities. All fractions containing product were combined and concentrated to give the pale brown solid. This solid was suspended in 50% DCM/Hex (100 mL) and sonicated for 10 min, filtered and dried under high vacuum. It was then dissolved in 50 mL of DMSO and precipitated by the addition of water (150 mL). This slurry was extracted with EtOAc (150 mL×3). Combined EtOAc layer was washed with water (150 mL×3), dried over Na₂SO₄, filtered and concentrated to dryness to give the title compound 4 as an off-white powder (1.55 g, 92%).

¹H NMR (600 MHz, DMSO-d₆): δ 12.51 (s, 1H, indole-NH), 8.68 (dd, J_{H-F}=11.3, 7.1 Hz, 1H, H₄), 8.55 (s, 1H, H₂), 8.53 (d, J=9.0 Hz, 1H, H_{4′}), 8.47 (d, J=7.8 Hz, 1H, —CONH), 8.17 (d, J=8.4 Hz, 1H, H_{8′}), 8.13 (d, J=9.0 Hz, 1H, H_{3′}), 7.74 (dd, J=8.4, 7.0 Hz, 1H, H_{7′}), 7.58 (d, J=7.0 Hz, 1H, H_{6′}), 4.18 (m, 1H, —CH(CH₃)₂), 1.22 (d, J=6.6 Hz, 6H, —CH(CH₃)₂).

¹⁹F NMR (375 MHz, DMSO-d₆): δ−144.50 (d, J=21.9 Hz, F₅), −155.32 (d, J=20.6 Hz, F₇), −169.45 (t, J=21.3 Hz, F₆).

¹³C NMR (150 MHz, DMSO): δ 166.74 (—CONH), 154.53 (C_{2′}), 147.46 (C_{5′}), 146.11 (dd, J_{C-F}=236.7, 11.4 Hz, C₇), 137.35 (ddd, J_{C-F}=248.6, 12.9, 4.5 Hz, C₅), 135.50 (ddd, J_{C-F}=240.3, 18.74, 12.2 Hz, C₆), 134.88 (C_{9′}), 133.67 (C_{4′}), 130.51 (C₂), 130.34 (C_{6′}), 128.58 (C_{7′}), 124.49 (C_{8′}), 123.15 (C_{10′}), 121.71 (dd, J_{C-F}=9.7, 2.0 Hz, C₉), 121.54 (dd, J_{C-F}=10.5, 5.1 Hz, C₈), 119.42 (C_{3′}), 116.04 (d, J_{C-F}=4.5 Hz, C₃), 104.24 (dd, J_{C-F}=20.3, 3.5 Hz, C₄), 41.03 (—CH(CH₃)₂), 22.32 (—CH(CH₃)₂).

HRMS: calculated for C₂₁H₁₇F₃N₃O [M+H]⁺: 384.1318, found: 384.1306.

TLC: 0.5 R_{f} in 75% EtOAc/Hex

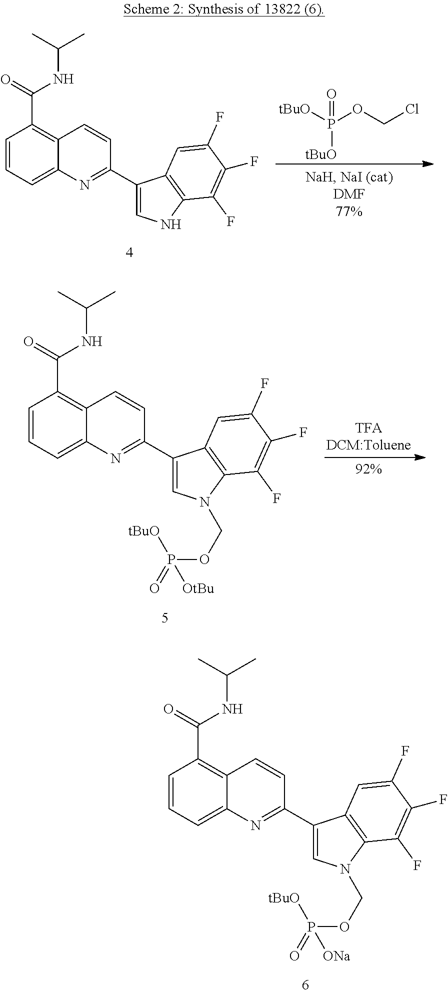

### Di-tert-butyl Ester Derivative (5)

NaH (190 mg, 60% suspension in oil, 4.7 mmol) was suspended in 10 mL of dry DMF at RT. The parent compound 4 (1.12 g, 2.9 mmol) was suspended in 3 mL of DMF, and added to the reaction mixture as a slurry, and rinsed over with 1 mL×2 DMF. Reaction mixture was stirred at RT for 10 min, at which point the solids had mostly dissolved. Catalytic amount of NaI (50 mg, 0.3 mmol) was added as solid, followed by the addition of chloromethyl phosphate reagent (neat, 850 μL, 3.3 mmol). After stirring at RT overnight, the reaction mixture was dumped into a stirred NH₄H₂PO₄—NH₃ buffer (1.5 M×50 mL), and rinsed over with DCM. The mixture was extracted with DCM (100 mL in 3 portions), washed once with NH₄Cl, and the aqueous was back extracted with DCM (20 mL). The combined organic was concentrated to dryness by rotary evaporation. The residue was triturated in MTBE (˜20 mL), and the mixture was aged at RT for 0.5 h. The mixture was filtered, and the filter cake was washed with 5 mL MTBE. The solid was dried under high vacuum for 1 h at RT to give the product as a pale pinkish-brown powder (1.363 g, 76.7% yield).

¹H NMR (500 MHz, DMSO-d₆): δ 8.78 (dd, J_{H-F}=11.0, 7.0 Hz, 1H, H₄), 8.64 (s, 1H, H₂), 8.61 (d, J=9.0 Hz, 1H, H_{4′}), 8.51 (d, J=7.8 Hz, 1H, —CONH), 8.23 (d, J=8.4 Hz, 1H, H_{8′}), 8.09 (d, J=9.0 Hz, 1H, H_{3′}), 7.78 (dd, J=8.4, 7.1 Hz, 1H, H_{7′}), 7.63 (dd, J=7.0, 1.1 Hz, 1H, H_{6′}), 6.12 (d, J_{H-P}=11.7 Hz, 2H, —CH₂P(O)(OtBu)₂), 4.28-4.12 (m, 1H, —CH(CH₃)₂), 1.33 (s, 18H, —OtBu×2), 1.23 (d, J=6.6 Hz, 6H, —CH(CH₃)₂).

¹⁹F NMR (470 MHz, DMSO-d₆): δ−142.36 (d, J=21.7 Hz, F₅), −156.21 (d, J=20.5 Hz, F₇), −167.78 (t, J=21.5 Hz, F₆).

HRMS: calculated for C₃₀H₃₆F₃N₃O₅P [M+H]⁺: 606.2339, found: 606.2342.

TLC: 0.2 R_{f} in 60% EtOAc/Hex

### 13822 (6)

The di-tert-butyl ester derivative 5 (1.33 g, 2.2 mmol) was suspended in a mixture of 7 mL DCM and 13 mL toluene in a 50 mL RBF cooled in a tap water bath. TFA (1.1 mL, 6.5 eq.) was added over 2 min. The resulting clear solution was stirred at RT for 6 h, at which point the reaction mixture had turned into a suspension. The mixture was added to a stirred mixture of MeOH (20 mL), water (30 mL), sat. Na₂CO₃ (30 mL), and EtOAc (50 mL). A few drops of 1% phenylphthalein in ethanol was added as pH indicator. Additional solid Na₂CO₃ was added as needed to keep the system basic. The solids slowly dissolved and the aqueous was visually uniformly turbid. Layers were separated and the organic layer was back-extracted with half-saturated Na₂CO₃. The aqueous layers were combined and purified on 120 g C18 in multiple 50-100 mL portions using a gradient of unbuffered MeOH-water. Pure fractions were concentrated to remove most of MeOH. The remaining aqueous solution, ˜200 mL, was passed through a 20 mL Amberlite IR120-Na ion exchange resin column, eluted with water, to remove any ammonium cation. The eluted liquid was concentrated to ˜20 mL, filtered through 0.45 um PTFE syringe filter, rinsed over with more water (10 mL×2). The filtrate was lyophilized overnight to give the title compound 6 as a white powder (92% Yield).

¹H NMR (500 MHz, D₂O): δ 8.37 (dd, J=9.0, 0.8 Hz, 1H, H_{4′}), 8.16 (s, 1H, H₂), 8.16 (dd, J_{H-F}=10.7, 7.1 Hz, 1H, H₄), 8.11 (d, J=8.5 Hz, 1H, H_{8′}), 7.80 (d, J=9.0 Hz, 1H, H_{3′}), 7.80 (dd, J=8.5, 7.1 Hz, 1H, H_{7′}), 7.67 (dd, J=7.1, 1.2 Hz, 1H, H_{6′}), 5.82 (d, J_{H-P}=7.1 Hz, 2H, —CH₂P(O)(OtBu)₂), 4.28 (m, 1H, —CH(CH₃)₂), 1.34 (d, J=6.6 Hz, 3H, —CH(CH₃)₂).

¹³C NMR (151 MHz, D₂O): δ 170.13 (—CONH), 154.38 (C_{2′}), 147.28 (m, C₇), 146.79 (C_{5′}), 138.53 (m, C₅), 136.9 (m, C₆), 134.15 (C_{4′}), 133.52 (C_{9′}), 133.33 (C₂), 129.92 (C_{6′}), 129.36 (C_{7′}), 125.21 (C_{8′}), 123.11 (C_{10′}), 122.29 (m, C₈), 121.16 (m, C₉), 120.67 (C_{3′}), 116.22 (m, C₃), 103.42 (m, C₄), 72.62 (—CH₂P(O)(OH)₂), 42.63 (—CH(CH₃)₂), 21.47 (—CH(CH₃)₂).

¹⁹F NMR (375 MHz, D₂O): δ−143.49 (d, J=21.2 Hz, F₅), −156.65 (d, J=20.2 Hz, F₇), −167.65 (t, J=20.5 Hz, F₆).

³¹P NMR (160 MHz, D₂O): δ 2.08 (s).

HRMS: calculated for C₂₂H₂₀F₃N₃O₅P [M+H]⁺: 494.1087, found: 494.1091, calc'd for C₂₂H₁₉F₃N₃NaO₅P [M+Na]⁺: 516.0907, found: 516.0911.

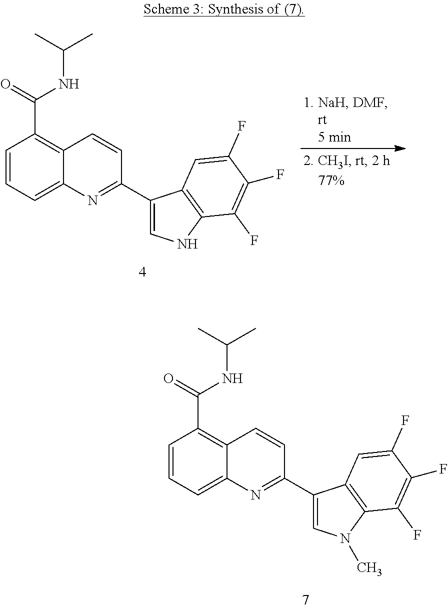 [SMILES: CC(C)NC(=O)c1cccc2nc(-c3c[nH]c4c(F)c(F)c(F)cc34)ccc12 ; CC(C)NC(=O)c1cccc2nc(-c3cn(C)c4c(F)c(F)c(F)cc34)ccc12]

### N-isopropyl-2-(5,6,7-trifluoro-1-methyl-1H-indol-3-yl)quinoline-5-carboxamide (7)

To a stirred solution of compound 4 (50 mg, 0.13 mmol) in DMF (1 mL), sodium hydride (60% in mineral oil, 9.0 mg, 0.21 mmol) was added at room temperature. Change in color from pale yellow to fluorescent yellow was observed. After stirring 5 min at room temperature, methyl iodide (11 μL, 0.17 mmol) was added. Immediately following the addition, color of the reaction mixture was changed from fluorescent yellow to pale yellow. Stirring was continued at room temperature for 2 h at which time TLC indicated completion of starting material. Mass spec analysis of the crude reaction mixture confirmed product formation. Reaction mixture was diluted with ethyl acetate (10 mL) and washed with water (5 mL×2). Ethyl acetate layer was dried over Na₂SO₄ and concentrated to dryness under reduced pressure to afford the crude. Crude material was column purified using a gradient of 0-80% EtOAc/Hex to give the title compound 7 as a white solid (40 mg, 77%).

¹H NMR (400 MHz, DMSO-d₆): δ 8.68 (ddd, J_{H-F}=11.0, 7.4, 1.4 Hz, 1H, H₄), 8.54 (d, J=9.0 Hz, 1H, H_{4′}), 8.48 (d, J=7.8 Hz, 1H, —CONH), 8.47 (s, 1H, H₂), 8.16 (d, J=8.4 Hz, 1H, H_{8′}), 8.0 (d, J=9.0 Hz, 1H, H_{3′}), 7.74 (dd, J=8.4, 7.3 Hz, 1H, H_{7′}), 7.58 (dd, J=7.1, 0.9 Hz, 1H, H_{6′}), 4.18 (m, 1H, —CH(CH₃)₂), 4.05 (d-like, 3H, N—CH₃), 1.22 (d, J=6.6 Hz, 6H, —CH(CH₃)₂).

HRMS: calculated for C₂₂H₁₈F₃N₃NaO [M+Na]⁺: 420.1294, found: 420.1280.

TLC: 0.6 R_{f} in 5% MeOH/DCM

The required coupling partner, 5,6,7-trifluoroindole 11 was synthesized from 2,3,4-trifluoroaniline 6 as shown in Scheme 4. Iodination of aniline 6 followed by carbamate protection gave the intermediate 8. Mono deprotection of the N,N-diethyl carbamate group followed by Sonogashira coupling with trimethylsilyl acetylene gave the indole precursor 10. Compound 10 was then converted in to 5,6,7-trifluoroindole 11 using sodium ethoxide.

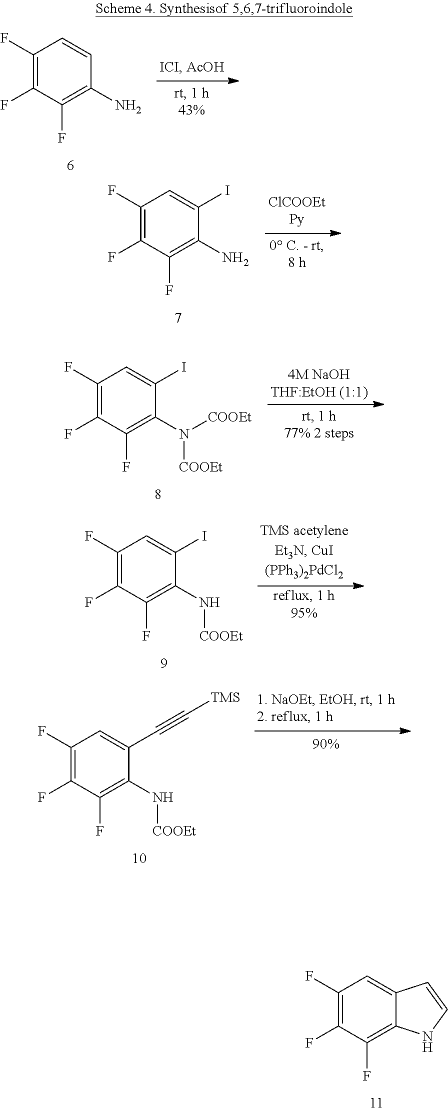 [SMILES: Nc1ccc(F)c(F)c1F ; Nc1c(I)cc(F)c(F)c1F ; CCOC(=O)N(C(=O)OCC)c1c(I)cc(F)c(F)c1F ; CCOC(=O)Nc1c(I)cc(F)c(F)c1F ; CCOC(=O)Nc1c(C#C[Si](C)(C)C)cc(F)c(F)c1F ; Fc1cc2cc[nH]c2c(F)c1F]

The construction of indolyl-quinoline scaffold was achieved by a slight modification of literature procedure a shown in Scheme 5. Thus, microwave irradiation of a mixture of 5-Bromoquinoline 12 and 5,6,7-trifluoroindole 11 in the presence of 4M HCl dioxane gave the key intermediate 13. The carboxylic acid 14 was obtained by bubbling carbon dioxide into the dianion generated by the sequential treatment of bromo intermediate 13 with methyl lithium and n-butyl lithium. The carboxylic acid 14 was then coupled with appropriate amine using hexafluorophosphate azabenzotriazole tetramethyl uronium (HATU) and N,N-diisopropylethylamine (DIPEA) to give the final compounds (4 and 15b-h, Scheme 5).

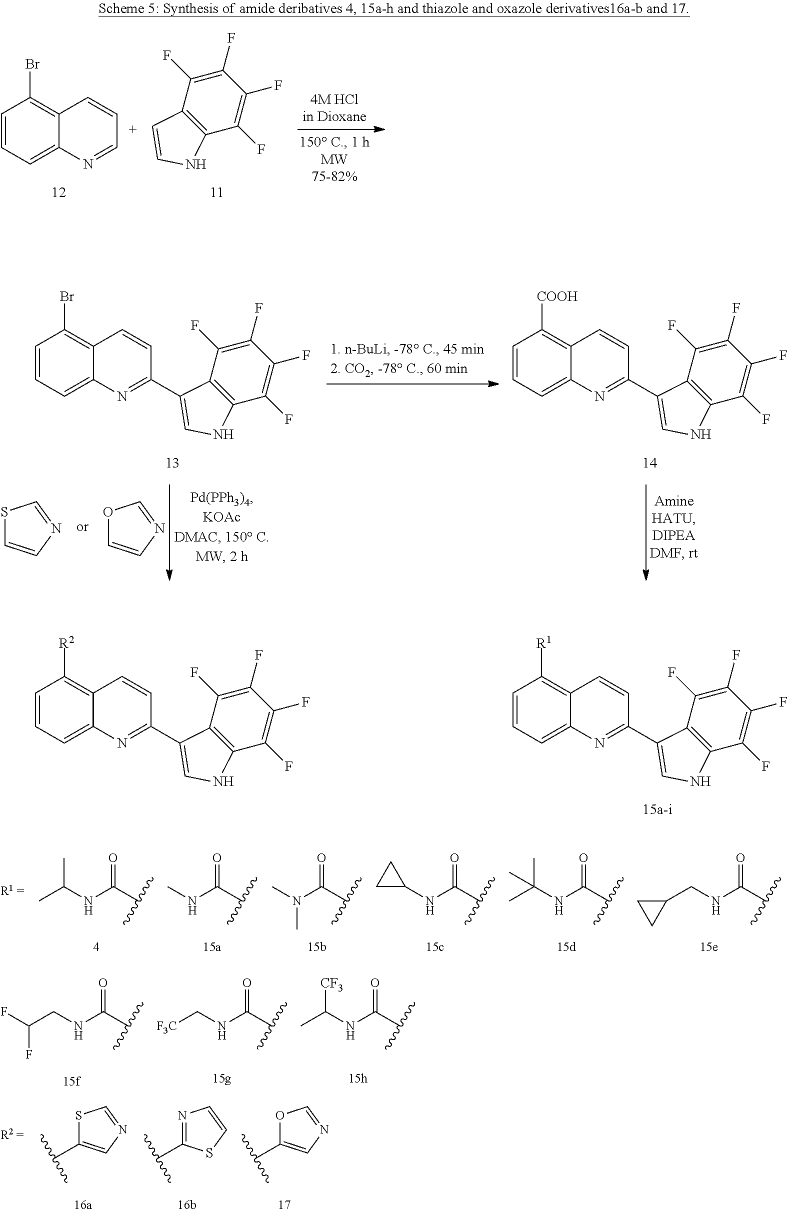

The key bromo intermediate 13 was also used to synthesize the thiazole and oxazole derivatives 16a-b and 17 (Scheme 5). Thus, thiazole and oxazole were coupled with the bromo compound 13 using Pd-catalyzed C—H activation protocol. In both cases, C-5 arylated regioisomers (16a and 17) were the major product. In the case of thiazole, the minor C-2 regioisomer (16b) was also isolated and evaluated for inhibitory activity. Following a similar sequence of reactions, 6-fluoroquinoline series target compounds 22a-b, 23a-d were synthesized starting from 5-bromo-6-fluoroquinoline as shown in Scheme 6. The starting material, 5-bromo-6-fluoroquinoline 19, was in turn synthesized via bromination of commercially available 6-flouroquinoline 18 (Scheme 6).

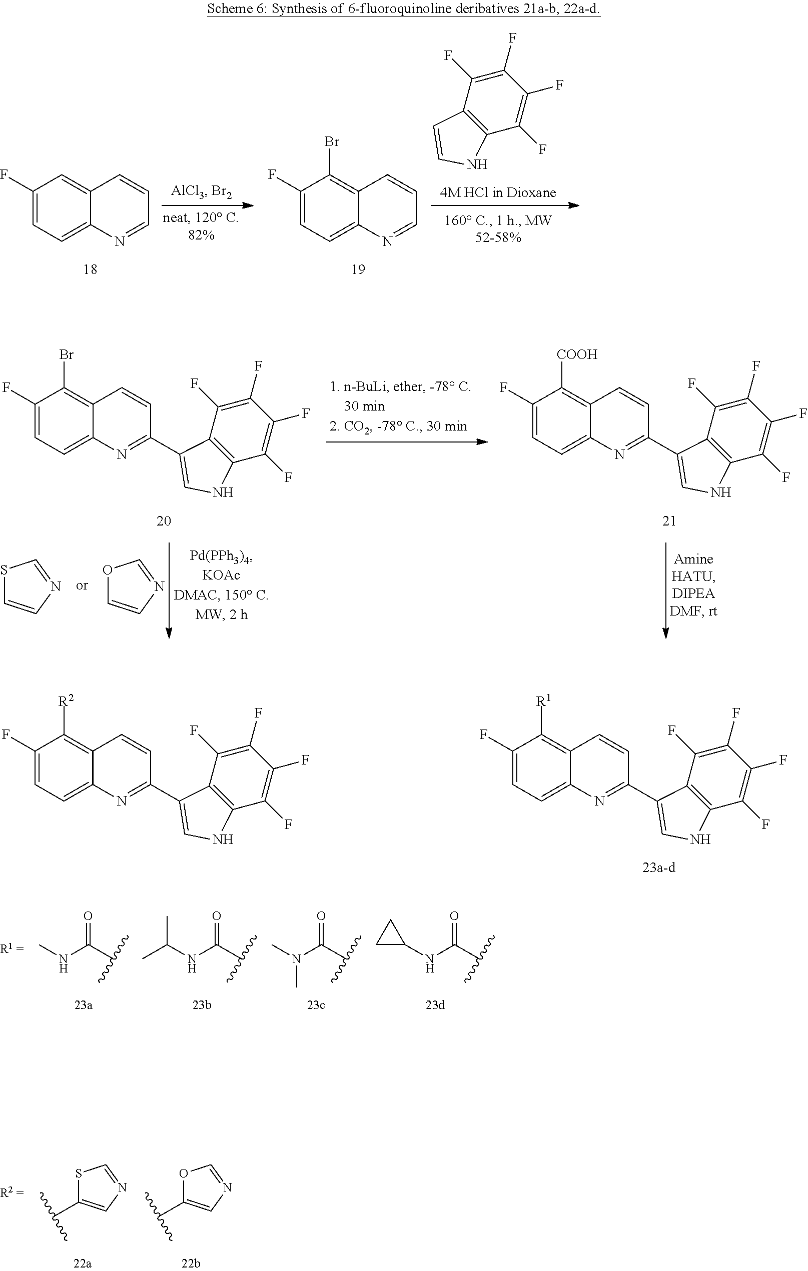

13789 (4) was prepared using molybdenum-mediated carbonylation of the key bromo intermediate 13 with isopropylamine as shown in Scheme 1. This route gave easy access to gram quantities of 4 without going through the low-yielding carboxylic acid route (Scheme 5). The N-methyl phosphate prodrug of 13789 (5) was synthesized as shown in Scheme 1. Thus, the N-alkylation of compound 4 with di-t-butyl-protected chloromethyl phosphate reagent using sodium hydride followed by the deprotection of t-butyl groups using TFA gave the N-methyl phosphate prodrug 5.

The following compounds were prepared using the methods described below.

General Chemistry Methods. ¹H and ¹³C NMR spectra were recorded with a Bruker Avance II 600 MHz spectrometer using a TCI cryoprobe, an Avance III 500 MHz spectrometer using a TXI inverse probe, or an Avance III 400 MHz spectrometer using a BBOF+ATM probe. All assignments were confirmed with the aid of two-dimensional ¹H-¹H (COSY) and/or ¹H-¹³C (HSQC) and ¹H-¹³C (HMBC) experiments and attached proton test (APT) using standard pulse programs. NMR data processing was performed with MestReNova software (MestreLab Research, ver. 12.0.3). The spectra were referenced to the corresponding solvent signals. LC-MS were recorded with an ESI ion source on an Agilent 6200 Time-of-Flight spectrometer coupled with Agilent 1200 series HPLC. Analytical thin-layer chromatography (TLC) was performed on aluminum plates precoated with silica gel 60E-254 as the adsorbent (EMD). The developed plates were air-dried, exposed to UV light and/or dipped in KMnO4 solution, and heated. Flash chromatography was performed on a BioTage Isolera instrument using HP-silica cartridges from BioTage or SiliCycle Inc. All reagents and solvents were obtained from commercial vendors and used as received. The purities of all final products were 95% or higher as determined by HPLC analysis. Compounds were analyzed using an Agilent 1100 HPLC and PDA detector at 254 nm with conditions 1: Agilent Zorbax SB-C8 column (3.0×150 mm, 5 μm) with a gradient of acetonitrile:0.1% formic acid from 5:95 to 95:5 over 5 min with a flow rate of 2 mL/min or conditions 2: Halo C18 column with gradient acetonitrile:5 mM ammonium acetate, pH 7, from 5:95 to 95:5 over 5 min with a flow rate of 2 mL/min.

2,3,4-trifluro-6-iodoaniline (7): 2,3,4-trifluoroaniline (20 mL, 189.4 mmol) was diluted in AcOH (600 mL). Under stirring, ICI (9.02 mL, 180.0 mmol) was added and the mixture was stirred at r.t. for 1 h. HPLC profile showed 90% of conversion. In conical flask containing mixture of sat. NaHCO₃ solution (250 mL)/EtOAc (250 mL), the reaction mixture was added drop-wise. pH of aqueous solution was checked and adjusted to basic with 1M NaOH solution. Then, resulting mixture was extracted with EtOAc (2×500 mL). Combined organic layer was washed with saturated NaHCO₃ solution, dried over Na₂SO₄ and concentrated to dryness under reduced pressure to afford the crude (dark oil, TLC: 0.5 Rf in 10% EtOAc/Hex). It was column purified using a gradient of 0-10% EtOAc/Hex to afford the title compound as dark waxy solid (22.0 g, 43% yield). Care must be taken while concentrating the column fractions as the product seems to sublime.

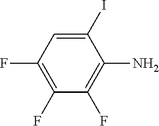 [SMILES: Nc1c(I)cc(F)c(F)c1F]

¹H NMR (600 MHz, CDCl₃): δ 7.33 (ddd, J=9.3, 7.6, 2.5 Hz, 1H), 4.07 (s, 2H).

¹³C NMR (150 MHz, CDCl₃): δ 143.20 (ddd, J_{C-F}=244.0, 10.8, 2.8 Hz), 139.98 (ddd, J_{C-F}=250.5, 16.1, 13.7 Hz), 138.48 (ddd, J_{C-F}=247.1, 12.6, 3.3 Hz), 133.43 (dd, J_{C-F}=11.2, 2.4 Hz), 119.96 (dd, J_{C-F}=20.1, 3.9 Hz), 73.21 (ddd, J_{C-F}=8.2, 4.5, 2.1 Hz).

HRMS calc'd for C₆H₄F₃IN [M+H]⁺: 273.9341, found: 273.9338.

Ethyl (ethoxycarbonyl)(2,3,4-trifluoro-6-iodophenyl) carbamate (8): Ethyl chloroformate (11.5 mL, 120.9 mmol) was added dropwise to a stirred solution of aniline 7 (22.0 g, 80.5 mmol) in pyridine (200 mL) at 0° C. Rate of addition was adjusted to maintain the pot temperature below 5° C. After stirring for 2 h at 0° C., the reaction mixture was diluted with EtOAc and washed with water. Organic layer was dried over Na₂SO₄ and concentrated to dryness under reduced pressure to afford compound 8 (33.0 g, dark brown oil which solidifies into a waxy solid upon storing). Crude material was used in the next step without any purification.

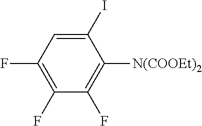

HRMS calc'd for C₁₂H₁₂F₃INO₄ [M+H]⁺: 417.9758, found: 417.9747, calc'd for C₁₂H₁₁F₃INNaO₄ [M+Na]⁺: 439.9577, found: 439.9569

TLC: 0.5 R_{f} in 20% EtOAc/Hex

Ethyl (2,3,4-trifluoro-6-iodophenyl) carbamate (9): To a stirred solution of carbamate 8 (33.0 g) in a mixture of THF (200 mL) and EtOH (200 mL), 4M NaOH (36 mL) was added and the reaction mixture was stirred at rt for 1 h. After this time, the reaction was complete as indicated by HPLC and Mass spec analysis. Ethanol was removed under reduced pressure and extracted with EtOAc (200 mL×2). Combined organic layers were dried over Na₂SO₄ and concentrated to dryness under reduced pressure to afford the crude. Crude material was column purified to give the title compound 9 as a pale yellow waxy solid (21.5 g, 77% yield).

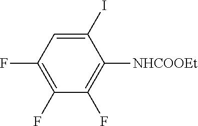

¹H NMR (600 MHz, CDCl₃): δ 7.51(ddd, J=9.8, 7.6, 2.4, 1H), 6.01 (s, 1H), 4.25 (q, J=7.1 Hz, 2H), 1.32 (t, J=7.1 Hz, 3H).

¹³C NMR (151 MHz, CDCl₃): δ 153.05, 149.38 (ddd, J_{C-F}=254.2, 10.9, 3.4 Hz), 146.70 (ddd, J_{C-F}=257.7, 14.8, 4.4 Hz), 140.09 (ddd, J_{C-F}=254.5, 15.5, 15.3 Hz), 124.67 (dd-like), 120.64 (dd, J_{C-F}=20.9, 3.9 Hz), 89.53, 61.94, 29.23, 13.96.

HRMS calc'd for C₉H₇F₃INNaO₂ [M+Na]³⁰ : 367.9371, found: 367.9392.

TLC: 0.5 R_{f} in 20% EtOAc/Hex. Note: there is no Rf difference between starting material and product.

Ethyl (2,3,4-trifluoro-6-((trimethylsilyl)ethynyl)phenyl) carbamate (10): To a stirred solution of carbamate 9 (21.5 g, 62.3 mmol) in CH₃CN (200 mL) under N₂ atmosphere was added, PdCl₂(PPh₃)₂ (4.4 g, 6.23 mmol), CuI (297 mg, 1.56 mmol), Et₃N (17.5 mL, 125 mmol) and TMS-acetylene (13.5 mL, 93.5 mmol). The reaction mixture was then refluxed for 1 h. TLC indicated completion of starting material. The reaction mixture was filtered through celite and washed with CH₃CN. Acetonitrile was removed by rota vapor and the crude diluted with EtOAc (150 mL×2) and washed with water (150 mL) and brine (100 mL). EtOAc layer was once again filtered through celite and concentrated to give the crude compound which was column purified to give the title compound 10 as a pale brown waxy solid (19.1 g, 97% yield).

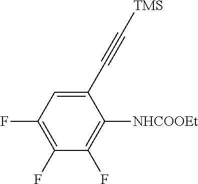

¹H NMR (600 MHz, CDCl₃): δ 7.8 (ddd, J=9.6, 7.4, 1.8 Hz, 1H), 6.25 (s, 1H), 4.25 (q, J=7.1 Hz, 2H), 1.31 (t, J=7.1 Hz, 3H), 0.26 (s, 9H)

¹³C NMR (150 MHz, CDCl₃): δ 153.7, 148.85 (ddd, J_{C-F}=249.4, 10.4, 3.7 Hz), 146.94 (ddd, J_{C-F}=254.6, 11.3, 4.5 Hz), 141.19 (dt, J_{C-F}=255.7, 15.2 Hz), 124.8 (dd, J_{C-F}=10.5, 3.4 Hz), 115.6 (br-d-like), 114.8 (dd, J_{C-F}=19.5, 3.5 Hz), 103.1, 97.9, 62.4, 14.6, 0.16 (3C).

HRMS calc'd for C₁₄H₁₇F₃NO₂Si [M+H]⁺: 316.0981, found: 316.0982.

TLC: 0.7 R_{f} in 20% EtOAc/Hex

5,6,7-trifluoro-1H-indole (11): To a stirred solution of NaOEt (16.4 g, 241 mmol) in EtOH (250 mL) at rt, a premade solution of carbamate 10 (19.0 g, 60.3 mmol) in EtOH (250 mL) was added under N₂ atmosphere. After the addition, stirring was continued for 1 h at rt until starting material is fully consumed as indicated by TLC and a new spot was formed at 0.2 R_{f}. The reaction mixture was then refluxed for 1 h at 85° C. At which point TLC indicated completion of reaction and formation of a non-polar spot at 0.5 R_{f}. EtOH was removed under reduced pressure and the crude was diluted with ether (225 mL) and washed with water (100 mL×2). Ether layer was concentrated to give the crude compound which was column purified to give the title compound 11 as a brown crystalline solid (8.9 g) and slightly impure material (0.32 g, 89% yield in total).

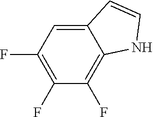 [SMILES: Fc1cc2cc[nH]c2c(F)c1F]

¹H NMR (600 MHz, CDCl₃): δ 8.30 (br-s, 1H), 7.27 (m, 1H), 7.17 (ddd, J=9.7, 6.5, 1.4 Hz, 1H), 6.56 (q, J=3.2 Hz, 1H)

¹⁹F NMR (470 MHz, CDCl₃): δ−145.0 (d, J=20.0 Hz), −156.9 (d, J=19.0 Hz), −168.9 (t, J=19.9 Hz).

¹³C NMR (150 MHz, CDCl₃): δ 147.12 (dd, J_{C-F}=239.5, 12.2 Hz), 138.10 (ddd, J_{C-F}=247.7, 13.5, 4.6 Hz), 136.61 (ddd, J_{C-F}=242.4, 18.7, 12.2 Hz), 126.26 (br-dd-like, J_{C-F}=3.2 Hz), 123.56 (dd, J_{C-F}=9.7, 5.0 Hz), 120.86 (d, J_{C-F}=8.2 Hz), 103.70 (dt, J_{C-F}=4.2, 1.9 Hz), 101.96 (dd, J_{C-F}=19.4, 3.9 Hz).

MS [(M−H)⁻]=170.0244

TLC: 0.6 R_{f} in 20% EtOAc/Hex

5-Bromo-2-(5,6,7-trifluoro-1H-indol-3-yl) quinoline (13): 5-Bromoquinoline (2.8 g, 13.44 mmol) and trifluoroindole 11 (1.0 g, 5.84 mmol) were mixed well in a 20 mL microwave vial. Mixed solids melted spontaneously and formed a brown gum. Then, 4M HCl solution in 1,4-dioxane (2.0 mL, 8.18 mmol) was added to the vial which resulted in a reddish orange suspension. Reaction vial was sealed and irradiated in a Biotage microwave reactor at 150° C. for 1 h without stirring. Resulted reddish orange brick-like solid was suspended in MeOH (90 mL) using sonication and the slurry was transferred to a separatory funnel and diluted with EtOAc (300 mL) and washed with saturated NaHCO₃ (150 mL). The aqueous layer was extracted with EtOAc (100 mL). Combined EtOAc layers were concentrated to dryness and the crude was purified by flash chromatography (Silica gel, a gradient from 30% DCM/Hexanes to 50% DCM and then 100% DCM) to give the title compound as a pale yellow solid (1.8 g, 80% yield).

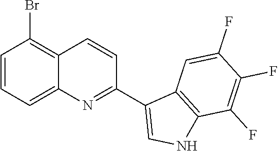 [SMILES: Fc1cc2c(-c3ccc4c(Br)cccc4n3)c[nH]c2c(F)c1F]

¹H NMR (500 MHz, DMSO-d₆): δ 12.57 (s, 1H), 8.65 (br-ddd-like, J=11.1, 7.2 Hz, 1H), 8.62 (s, 1H), 8.40 (d, J=9.0 Hz, 1H), 8.23 (d, J=9.0 Hz, 1H), 8.14 (d, J=8.4 Hz, 1H), 7.85 (d, J=7.5 Hz, 1H), 7.66 (t, J=8.0 Hz, 1H).

HRMS calc'd for C₁₇H₉BrF₃N₂ [M+H]⁺: 376.9896 and 378.9877, found: 376.9894 and 378.9875.

TLC: 0.3 R_{f} in 20% EtOAc/Hex

2-(5,6,7-Trifluoro-1H-indol-3-yl)quinoline-5-carboxylic acid (14): n-BuLi (1.6M in hexanes, 9.0 mL, 14.31 mmol) was added dropwise to a stirred solution of 5-bromo-2-(5,6,7-trifluoro-1H-indol-3-yl) quinoline (13, 1.8 g, 4.77 mmol) in dry diethyl ether (200 mL) at −78° C. over 10 min period. The resulting dark yellow slurry was stirred at −78° C. for 30 min. A stream of carbon dioxide gas was bubbled through the solution at −78° C. for 30 min. Reaction mixture was then poured into freshly crushed dry ice and carefully quenched with water (50 mL). pH of the biphasic mixture was adjusted to 4 using 1N HCl. The resulting biphasic mixture was extracted with EtOAc (100 mL×3). Combined EtOAc layer was dried over Na₂SO₄, filtered and concentrated to dryness to give the crude product (1.5 g). TLC analysis of the crude indicated a mixture of product and unreacted starting material and this material was used as such in the next step without any further purification.

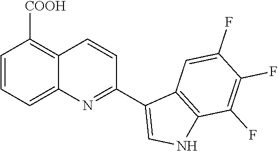 [SMILES: O=C(O)c1cccc2nc(-c3c[nH]c4c(F)c(F)c(F)cc34)ccc12]

N-isopropyl-2-(5,6,7-trifluoro-1H-indol-3-yl)quinoline-5-carboxamide (4 (13789)): To a stirred solution of carboxylic acid 14 (85 mg, 0.24 mmol) in DMF (3 mL) at 22° C. was added DIPEA (0.12 mL, 0.69 mmol), isopropylamine (0.1 mL, 1.22 mmol) and HATU (89 mg, 0.24 mmol). The reaction mixture was then stirred at 22° C. for overnight. DMF was removed under high vacuum and the crude was the purified by flash chromatography (Biotage, a gradient from 0 to 4% MeOH/DCM). The isolated solid (60 mg) was further purified by trituration with DCM (6 mL) to yield the compound 4 as off-white solid (16 mg, 17% yield).

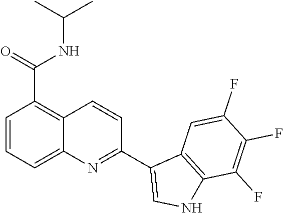 [SMILES: CC(C)NC(=O)c1cccc2nc(-c3c[nH]c4c(F)c(F)c(F)cc34)ccc12]

¹H NMR (600 MHz, CD₃OD): δ 8.53 (dd, J=8.9, 0.9 Hz, 1H), 8.50 (ddd, J=11.3, 7.0, 1.8 Hz, 1H), 8.19 (s, 1H), 8.16 (d, J=8.4 Hz, 1H), 7.96 (d, J=8.9 Hz, 1H), 7.73 (dd, J=8.5, 7.1 Hz, 1H), 7.60 (dd, J=7.1, 1.3 Hz, 1H), 4.31 (m, J=6.6 Hz, 1H), 1.32 (d, J=6.6 Hz, 6H).

¹³C NMR (150 MHz, CD₃OD): δ 170.56, 156.54, 149.41, 148.40 (dd, J=237.6, 11.6 Hz), 139.16 (ddd, J=247.8, 13.1, 4.5 Hz), 137.8 (ddd, J=241.1, 18.7, 12.0 Hz), 135.90, 134.92, 131.91, 130.12 (d, J=3.0 Hz), 129.72, 125.67, 124.84, 123.52 (d, J=9.9 Hz), 123.16 (dd, J=10.5, 4.8 Hz), 120.81, 118.14 (d, J=4.7 Hz), 105.13 (dd, J=20.8, 3.8 Hz), 43.26, 22.60 (2C).

HRMS calc'd for C₂₁H₁₇F₃N₃O [M+H]⁺: 384.1318, found: 384.1300.

TLC: 0.5 R_{f} in 75% EtOAc/Hex

### N,N-dimethyl-2-(5,6,7-trifluoro-1H-indol-3-yl)quinoline-5-carboxamide (15b)

Compound 15b was obtained from carboxylic acid 14 (175 mg, 0.51 mmol), DIPEA (0.36 mL, 2.04 mmol), dimethylamine (2 M in THF, 0.51 mL, 1.02 mmol), HATU (387 mg, 1.02 mmol) and DMF (4 mL) using the procedure as that described to obtain 4. The crude product was the purified by flash chromatography (Biotage, a gradient from 0 to 3% MeOH/DCM). The isolated solid (120 mg) was further purified by trituration with DCM (5 mL) to yield the compound 15b as off-white solid (55 mg, 29% yield).

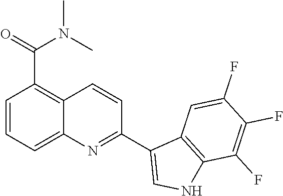 [SMILES: CN(C)C(=O)c1cccc2nc(-c3c[nH]c4c(F)c(F)c(F)cc34)ccc12]

¹H NMR (600 MHz, DMSO-d₆): δ 8.67 (br-ddd-like, J=11.0, 7.3 Hz, 1H), 8.56 (s, 1H), 8.14 (dt, J=8.4, 0.9 Hz, 1H), 8.12 (d, J=9.0 Hz, 1H), 8.04 (dd, J=8.9, 0.8 Hz, 1H), 7.76 (dd, J=8.4, 7.1 Hz, 1H), 7.43 (dd, J=7.0, 1.0 Hz, 1H), 3.14 (s, 3H), 2.78 (s, 3H).

¹³C NMR (150 MHz, DMSO-d₆): δ 168.5, 154.8, 147.3, 146.1 (dd, J_{C-F}=236.8, 11.3 Hz), 138.3-136.4 (m), 136.5-134.7 (m), 134.9, 133.1, 130.6 (d, J_{C-F}=2.7 Hz), 129.3, 129.0, 123.1, 122.3, 121.7 (d, J_{C-F}=9.2 Hz), 121.5 (dd, J_{C-F}=10.6, 5.1 Hz), 119.7, 116.0 (d, J_{C-F}=4.6 Hz), 104.2 (d, J_{C-F}=20.3, 3.7 Hz), 38.4, 34.3.

HRMS calc'd for C₂₀H₁₅F₃N₃O [M+H]⁺: 370.1167, found: 370.1168.

TLC: 0.3 R_{f} in 5% MeOH/DCM

N-Cyclopropyl-2-(5,6,7-trifluoro-1H-indol-3-yl)quinoline-5-carboxamide (15c (13795)): Compound 15c was obtained from carboxylic acid 14 (175 mg, 0.51 mmol), DIPEA (0.36 mL, 2.04 mmol), cyclopropylamine (71 μL, 1.02 mmol), HATU (387 mg, 1.02 mmol) and DMF (4 mL) using the procedure as that described to obtain 4. The crude product was the purified by flash chromatography (Biotage, a gradient from 0 to 4% MeOH/DCM). The isolated solid was re-purified by another flash chromatography (Biotage, a gradient from 10 to 80% EtOAc/Hexanes) to yield the compound 15c as pale yellow solid (85 mg, 44% yield).

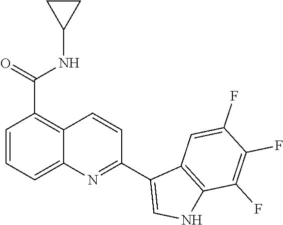 [SMILES: O=C(NC1CC1)c1cccc2nc(-c3c[nH]c4c(F)c(F)c(F)cc34)ccc12]

¹H NMR (600 MHz, DMSO-d₆): δ 12.51 (s, 1H), 8.67 (ddd, J=11.3, 7.2, 1.6 Hz, 1H), 8.64 (d, J=4.4 Hz, 1H), 8.58-8.53 (m, 2H), 8.17 (dt, J=8.4, 1.1 Hz, 1H), 8.13 (d, J=9.0 Hz, 1H), 7.73 (dd, J=8.4, 7.1 Hz, 1H), 7.58 (dd, J=7.1, 1.2 Hz, 1H), 3.01-2.91 (m, 1H), 0.80-0.69 (m, 2H), 0.66-0.56 (m, 2H).

¹³C NMR (150 MHz, DMSO-d₆): δ 168.8, 154.6, 147.5, 146.1 (dd, J_{C-F}=236.7, 11.3 Hz), 138.5-136.4 (m), 136.5-134.5 (m), 134.3, 133.7, 130.6 (br-d-like), 130.5, 128.5, 124.6, 123.1, 121.7 (d, J_{C-F}=9.7 Hz), 121.5 (dd, J_{C-F}=10.5, 5.0 Hz), 119.5, 116.0 (d, J_{C-F}=4.6 Hz), 104.2 (dd, J_{C-F}=20.3, 3.6 Hz), 23.0, 5.8 (2C).

HRMS calc'd for C₂₁H₁₅F₃N₃O [M+H]⁺: 382.1167, found: 382.1174.

TLC: 0.4 R_{f} in 5% MeOH/DCM

### N-(tert-Butyl)-2-(5,6,7-trifluoro-1H-indol-3-yl)quinoline-5-carboxamide (15d (13806))

Compound 15d was obtained from carboxylic acid 14 (60 mg, 0.17 mmol), DIPEA (0.12 mL, 0.68 mmol), t-butylamine (40 μL, 0.35 mmol), HATU (133 mg, 0.35 mmol) and DMF (3 mL) using the procedure as that described to obtain 4. The crude product was the purified by flash chromatography (Biotage, a gradient from 0 to 50% EtOAc/Hexanes) to yield the compound 15d as pale yellow solid (7 mg, 10% yield).

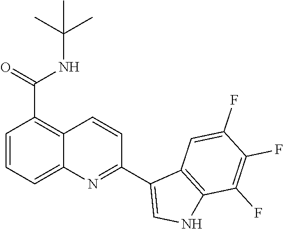 [SMILES: CC(C)(C)NC(=O)c1cccc2nc(-c3c[nH]c4c(F)c(F)c(F)cc34)ccc12]

¹H NMR (600 MHz, DMSO-d₆): δ 12.51 (s, 1H), 8.73-8.62 (m, 1H), 8.54 (s, 1H), 8.44 (d, J=8.8 Hz, 1H), 8.23-8.07 (m, 3H), 7.72 (t, J=7.7 Hz, 1H), 7.53 (d, J=7.0 Hz, 1H), 1.45 (s, 9H).

¹³C NMR (150 MHz, DMSO-d₆): δ 167.6, 154.5, 147.4, 146.1 (dd, J_{C-F}=236.5, 11.3 Hz), 138.5-136.4 (m), 136.5-134.6 (m), 135.9, 133.6, 130.5, 130.5, 129.9, 128.6, 124.3, 123.1, 121.7 (d, J_{C-F}=9.1 Hz), 121.5 (dd-like, J_{C-F}=6.2 Hz), 119.4, 116.1, 104.5-104.1 (m), 51.1, 28.6 (3C).

HRMS calc'd for C₂₂H₁₉F₃N₃O [M+H]⁺: 398.1480, found: 398.1464.

TLC: 0.5 R_{f} in 50% EtOAc/Hexanes

N-(Cyclopropylmethyl)-2-(5,6,7-trifluoro-1H-indol-3-yl)quinoline-5-carboxamide (15e (13807)): Compound 15e was obtained from carboxylic acid 14 (85 mg, 0.25 mmol), DIPEA (0.17 mL, 1.0 mmol), cyclopropanemethylamine (45 μL, 0.50 mmol), HATU (190 mg, 0.50 mmol) and DMF (3 mL) using the procedure as that described to obtain 4. The crude product was the purified by flash chromatography (Biotage, a gradient from 0 to 4% MeOH/DCM). The isolated solid was further purified by trituration with DCM (3 mL) to yield the compound 15e as off-white solid (35 mg, 35% yield).

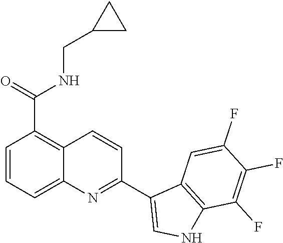 [SMILES: O=C(NCC1CC1)c1cccc2nc(-c3c[nH]c4c(F)c(F)c(F)cc34)ccc12]

¹H NMR (600 MHz, DMSO-d₆): δ 12.51 (s, 1H), 8.77-8.63 (m, 2H), 8.62-8.52 (m, 2H), 8.18 (dt, J=8.4, 1.1 Hz, 1H), 8.14 (d, J=9.0 Hz, 1H), 7.75 (dd, J=8.4, 7.1 Hz, 1H), 7.61 (dd, J=7.1, 1.2 Hz, 1H), 3.24 (t, J=6.2 Hz, 2H), 1.15-1.06 (m, 1H), 0.54-0.43 (m, 2H), 0.35-0.24 (m, 2H).

¹³C NMR (150 MHz, DMSO-d₆): δ 167.5, 154.6, 147.5, 146.1 (dd, J_{C-F}=236.8, 11.4 Hz), 138.3-136.4 (m), 136.5-134.6 (m), 134.7, 133.7, 130.6 (d, J_{C-F}=2.6 Hz), 130.5, 128.6, 124.5, 123.2, 121.8-121.6 (m), 121.5 (dd, J_{C-F}=10.5, 5.0 Hz), 119.4, 116.0 (d, J_{C-F}=4.6 Hz), 104.2 (dd, J_{C-F}=20.3, 3.6 Hz), 43.3, 11.0, 3.3 (2C).

HRMS calc'd for C₂₂H₁₇F₃N₃O [M+H]⁺: 396.1324, found: 396.1318.

TLC: 0.4 R_{f} in 10% MeOH/DCM

N-(2,2-Difluoroethyl)-2-(5,6,7-trifluoro-1H-indol-3-yl)quinoline-5-carboxamide (15f (13808)): Compound 15f was obtained from carboxylic acid 14 (85 mg, 0.25 mmol), DIPEA (0.17 mL, 1.0 mmol), 2,2-difluoroethylamine (36 μL, 0.50 mmol), HATU (190 mg, 0.50 mmol) and DMF (3 mL) using the procedure as that described to obtain 4. The crude product was the purified by flash chromatography (Biotage, a gradient from 0 to 4% MeOH/DCM). The isolated solid was further purified by trituration with DCM (3 mL) to yield the compound 15f as off-white solid (55 mg, 54% yield).

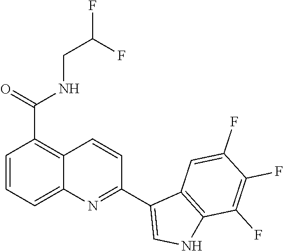 [SMILES: O=C(NCC(F)F)c1cccc2nc(-c3c[nH]c4c(F)c(F)c(F)cc34)ccc12]

¹H NMR (600 MHz, DMSO-d₆): δ 12.52 (s, 1H), 9.02 (t, J=6.0 Hz, 1H), 8.68 (ddd, J=11.3, 7.1, 1.6 Hz, 1H), 8.57 (s, 1H), 8.55 (d, J=9.1 Hz, 1H), 8.21 (d, J=8.4 Hz, 1H), 8.15 (d, J=9.0 Hz, 1H), 7.77 (dd, J=8.4, 7.1 Hz, 1H), 7.65 (dd, J=7.1, 1.2 Hz, 1H), 6.24 (tt, J_{H-F}=55.9 Hz, J=3.9 Hz, 1H), 3.77 (tdd, J_{H-F}=15.8 Hz, J=6.0, 3.9 Hz, 2H).

¹³C NMR (150 MHz, DMSO-d₆): δ 168.3, 154.7, 147.5, 146.1 (dd, J_{C-F}=236.7, 11.3 Hz), 138.3-136.4 (m), 136.5-134.6 (m), 133.6, 133.5, 131.0, 130.7 (d, J_{C-F}=2.6 Hz), 128.6, 124.8, 123.1, 121.7 (d, J_{C-F}=9.6 Hz), 121.5 (dd, J_{C-F}=10.4, 5.1 Hz), 119.6, 116.0 (d, J_{C-F}=4.6 Hz), 114.6 (t, J_{C-F}=240.0 Hz), 104.2 (dd, J_{C-F}=20.1, 3.6 Hz), 41.4 (t, J_{C-F}=25.8 Hz).

HRMS calc'd for C₂₀H₁₃F₅N₃O [M+H]⁺: 406.0979, found: 406.0984.

TLC: 0.4 R_{f} in 10% MeOH/DCM

2-(5,6,7-Trifluoro-1H-indol-3-yl)-N-(2,2,2-trifluoroethyl)quinoline-5-carboxamide (15g): Compound 15g was obtained from carboxylic acid 14 (200 mg, 0.58 mmol), DIPEA (0.41 mL, 2.34 mmol), 2,2,2-trifluoroethylamine (92 μL, 1.17 mmol), HATU (444 mg, 1.17 mmol) and DMF (3 mL) using the procedure as that described to obtain 4. The reaction mixture was diluted with water (25 mL) and extracted with EtOAc (40 mL×2). Combined organic layer was concentrated to dryness and purified by flash chromatography (Biotage, a gradient from 0 to 50% EtOAc/Hexanes) to yield the compound 15g as off-white solid (70 mg, 28% yield).

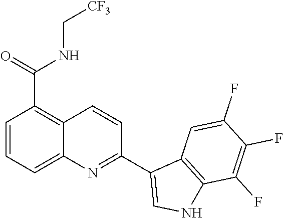 [SMILES: O=C(NCC(F)(F)F)c1cccc2nc(-c3c[nH]c4c(F)c(F)c(F)cc34)ccc12]

¹H NMR (600 MHz, DMSO-d₆): δ 12.53 (s, 1H), 9.33 (t, J=6.3 Hz, 1H), 8.68 (ddd, J=11.3, 7.1, 1.6 Hz, 1H), 8.57 (s, 1H), 8.49 (d, J=9.0 Hz, 1H), 8.24 (d, J=8.3 Hz, 1H), 8.18 (d, J=9.0 Hz, 1H), 7.79 (dd, J=8.4, 7.1 Hz, 1H), 7.66 (dd, J=7.2, 1.2 Hz, 1H), 4.24-4.14 (m, 2H).

¹³C NMR (150 MHz, DMSO-d₆): δ 168.3, 154.8, 147.5, 146.1 (dd, J_{C-F}=236.7, 11.4 Hz), 138.3-136.5 (m), 136.5-134.7 (m), 133.2, 133.1, 131.2, 130.7 (d, J_{C-F}=2.6 Hz), 128.6, 124.9, 124.9 (q, J_{C-F}=279.4 Hz), 123.1, 121.7 (d, J_{C-F}=9.5 Hz), 121.5 (dd, J_{C-F}=10.5, 5.0 Hz), 119.8, 116.0 (d, J_{C-F}=4.7 Hz), 104.3 (dd, J_{C-F}=20.3, 3.8 Hz), 40.10 (q, J_{C-F}=33.4 Hz).

HRMS calc'd for C₂₀H₁₂F₆N₃O [M+H]⁺: 424.0885, found: 424.0871.

TLC: 0.5 R_{f} in 50% EtOAc/Hexanes

2-(5,6,7-Trifluoro-1H-indol-3-yl)-N-(1,1,1-trifluoropropan-2-yl)quinoline-5-carboxamide (15h): Compound 15h was obtained from carboxylic acid 14 (200 mg, 0.58 mmol), DIPEA (0.41 mL, 2.34 mmol), 1-(trifluoromethyl)ethylamine (101 μL, 1.17 mmol), HATU (444 mg, 1.17 mmol) and DMF (3 mL) using the procedure as that described to obtain 4. The reaction mixture was diluted with water (25 mL) and extracted with ether (40 mL×2). Combined organic layer was concentrated to dryness and purified by flash chromatography (Biotage, a gradient from 0 to 50% EtOAc/Hexanes) to yield the compound 15h as off-white solid (50 mg, 20% yield).

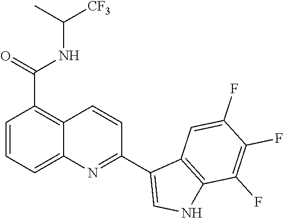 [SMILES: CC(NC(=O)c1cccc2nc(-c3c[nH]c4c(F)c(F)c(F)cc34)ccc12)C(F)(F)F]

¹H NMR (600 MHz, DMSO-d₆): δ 12.52 (s, 1H), 9.20 (d, J=8.8 Hz, 1H), 8.68 (dd, J=11.1, 7.1 Hz, 1H), 8.57 (s, 1H), 8.45 (dd, J=9.0, 0.9 Hz, 1H), 8.23 (d, J=8.3 Hz, 1H), 8.17 (d, J=9.0 Hz, 1H), 7.79 (dd, J=8.4, 7.1 Hz, 1H), 7.63 (dd, J=7.1, 1.2 Hz, 1H), 4.93 (m, 1H), 1.39 (d, J=7.1 Hz, 3H).

¹³C NMR (150 MHz, DMSO-d₆): δ 167.7, 154.7, 147.4, 146.1 (dd, J_{C-F}=236.8, 11.3 Hz), 138.4-136.4 (m), 136.5-134.6 (m), 133.4, 133.2, 131.0, 130.7 (d, J_{C-F}=2.7 Hz), 128.7, 125.9 (q, J_{C-F}=282.1 Hz), 124.9, 123.0, 121.7 (d, J_{C-F}=9.6 Hz), 121.5 (dd, J_{C-F}=10.7, 5.1 Hz), 119.7, 115.9 (m), 104.2 (dd, J_{C-F}=20.0, 3.6 Hz), 45.8 (q, J_{C-F}=30.5 Hz), 13.3.

HRMS calc'd for C₂₁H₁₄F₆N₃O [M+H]⁺: 438.1041, found: 438.1016.

TLC: 0.5 R_{f} in 50% EtOAc/Hexanes

5-(2-(5,6,7-Trifluoro-1H-indol-3-yl)quinolin-5-yl)thiazole (16a (13793)) and 2-(2-(5,6,7-Trifluoro-1H-indol-3-yl)quinolin-5-yl)thiazole (16b (13801)): In a microwave vial, thiazole (94 μL, 1.32 mmol) and Pd(PPh₃)₄ (16 mg, 0.013 mmol) were added to the solution of bromo compound 13 (100 mg, 0.26 mmol) and KOAc (78 mg, 0.79 mmol) in DMAc (1.1 mL). The microwave vial was then sealed and irradiated in a Biotage microwave reactor at 150° C. for 2 h. Reaction mixture was diluted with EtOAc (30 mL) and washed with water (15 mL). Organic layer was concentrated to dryness and purified by flash chromatography (Biotage, a gradient from 0-50% EtOAc/Hexanes). Two regioisomers (C-2 and C-5 substituted thiazole derivatives) were isolated and both compounds were further purified by trituration with 50% DCM/Hexanes. The non-polar compound was identified as the C-2 regioisomer (off-white solid, 5 mg) and polar compound was identified as the C-5 regioisomer (off-white solid, 33 mg, 38% yield for both isomers).

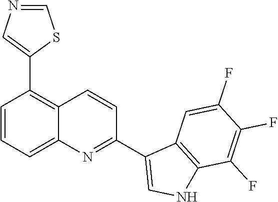 [SMILES: Fc1cc2c(-c3ccc4c(-c5cncs5)cccc4n3)c[nH]c2c(F)c1F]

Data for 16a: ¹H NMR (600 MHz, DMSO-d₆): δ 9.31 (d, J=0.8 Hz, 1H), 8.69 (br-dd, J=11.0, 7.3 Hz, 1H), 8.57 (s, 1H), 8.34 (dd, J=9.0, 0.9 Hz, 1H), 8.19 (dt, J=8.4, 0.9 Hz, 1H), 8.17 (d, J=0.8 Hz, 1H), 8.14 (d, J=9.0 Hz, 1H), 7.80 (dd, J=8.4, 7.1 Hz, 1H), 7.63 (dd, J=7.2, 1.2 Hz, 1H).

¹³C NMR (150 MHz, DMSO-d₆): δ 155.0, 154.7, 147.8, 146.1 (dd, J_{C-F}=236.8, 11.3 Hz), 142.6, 138.3-136.4 (m), 136.6-134.7 (m), 134.5, 133.0, 130.7 (d, J_{C-F}=2.6 Hz), 129.6, 129.2, 128.2, 127.8, 124.1, 121.7 (d, J_{C-F}=9.5 Hz), 121.6 (dd, J_{C-F}=10.8, 5.0 Hz), 119.8, 115.9 (d, J_{C-F}=4.1, 11.3 Hz), 104.2 (dd, J_{C-F}=20.3, 3.6 Hz).

HRMS calc'd for C₂₀H₁₁F₃N₃S [M+H]⁺: 382.0626, found: 382.0628.

TLC: 0.2 R_{f} in 50% EtOAc/Hexanes

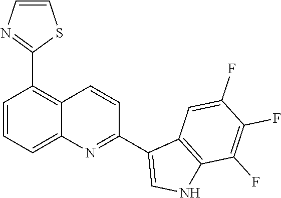 [SMILES: Fc1cc2c(-c3ccc4c(-c5nccs5)cccc4n3)c[nH]c2c(F)c1F]

Data for 16b: ¹H NMR (600 MHz, DMSO-d₆): δ 12.54 (s, 1H), 9.23 (d, J=9.1 Hz, 1H), 8.70 (dd, J=11.2, 7.0 Hz, 1H), 8.58 (s, 1H), 8.24 (d, J=8.2 Hz, 1H), 8.19 (d, J=9.1 Hz, 1H), 8.13 (d, J=3.3 Hz, 1H), 7.96 (d, J=3.3 Hz, 1H), 7.93 (d, J=7.2 Hz, 1H), 7.83 (t, J=7.8, 1.2 Hz, 1H).

¹³C NMR (150 MHz, DMSO-d₆): δ 166.0, 154.8, 148.0, 146.2 (dd, J_{C-F}=236.6, 11.1 Hz), 143.9, 138.3-136.4 (m), 136.6-134.7 (m), 134.0, 130.9, 130.7 (br-s), 129.9, 129.2, 127.4, 122.9, 121.8-121.7 (m), 121.7-121.5 (m), 121.4, 120.0, 115.9 (br-s), 104.3 (br-dd-like, J_{C-F}=20.2 Hz).

HRMS calc'd for C₂₀H₁₁F₃N₃S [M+H]⁺: 382.0626, found: 382.0577.

TLC: 0.5 R_{f} in 50% EtOAc/Hexanes

5-(2-(5,6,7-Trifluoro-1H-indol-3-yl)quinolin-5-yl)oxazole (17): In a microwave vial, oxazole (87 μL, 1.32 mmol) and Pd(PPh₃)₄ (16 mg, 0.013 mmol) were added to the solution of bromo compound 13 (100 mg, 0.26 mmol) and KOAc (78 mg, 0.79 mmol) in DMAc (1.2 mL). The microwave vial was then sealed and irradiated in a Biotage microwave reactor at 150° C. for 2 h. Reaction mixture was diluted with EtOAc (30 mL) and washed with water (15 mL). Organic layer was concentrated to dryness and purified by flash chromatography (Biotage, a gradient from 0-50% EtOAc/Hexanes) to yield compound 17 as off-white solid (33 mg, 34% yield).

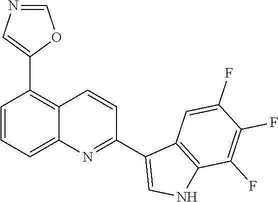 [SMILES: Fc1cc2c(-c3ccc4c(-c5cnco5)cccc4n3)c[nH]c2c(F)c1F]

¹H NMR (600 MHz, DMSO-d₆): δ 12.54 (s, 1H), 8.69 (dd, J=11.2, 7.4 Hz, 1H), 8.63 (s, 1H), 8.63-8.56 (m, 2H), 8.23-8.11 (m, 2H), 7.87-7.74 (m, 3H).

¹³C NMR (150 MHz, DMSO-d₆): δ 155.2, 152.9, 149.4, 148.3, 146.6 (dd, J_{C-F}=236.9, 11.0 Hz), 138.8-136.9 (m), 137.0-135.1 (m), 133.5, 131.2 (d, J_{C-F}=2.6 Hz), 130.5, 129.8, 125.7, 125.6, 125.2, 122.9, 122.2 (br-dd-like, J_{C-F}=9.4 Hz), 122.0 (br-dd-like, J_{C-F}=10.6, 5.3 Hz), 120.3, 116.3(br-d, J_{C-F}=4.3 Hz), 104.7 (br-dd-like, J_{C-F}=20.3, 3.0 Hz).

HRMS calc'd for C₂₀H₁₁F₃N₃S [M+H]⁺: 366.0854, found: 366.0858.

TLC: 0.2 R_{f} in 50% EtOAc/Hexanes

5-Bromo-6-fluoroquinoline (19): A mixture of 6-fluoroquinoline 18 (5.0 g, 34.0 mmol) and aluminium trichloride (13.6 g, 101.9 mmol) was immersed in a preheated oil bath at 120° C. Bromine (1.75 mL, 34.0 mmol) was added dropwise at 120° C. over 1 h period. After the addition, stirring was continued at 120° C. for 2 h. Reaction mixture was cooled down to rt and carefully quenched with a mixture of methanol and water (250 mL, 1:1 ratio) and stirred at rt for 1 h. Methanol was then removed by rotavapor and extracted the aqueous layer with DCM (150 mL×2). Combined organic layer was concentrated to dryness and purified by flash column chromatography (Biotage, a gradient from 0-20% EtOAc/Hexanes) to give compound 19 as pale brown solid (6.2 g, 80% yield).

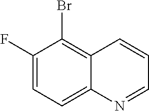 [SMILES: Fc1ccc2ncccc2c1Br]

¹H NMR (500 MHz, DMSO-d₆): δ 8.98 (dd, J=4.2, 1.6, 1H), 8.57-8.49 (m, 1H), 8.15 (ddd, J=9.3, 5.3, 0.8 Hz, 1H), 7.86 (t, J=8.9 Hz, 1H), 7.75 (ddd, J=8.6, 4.2, 0.8 Hz, 1H).

¹³C NMR (150 MHz, CDCl₃): δ 157.4 (d, J_{C-F}=249.0 Hz), 150.2 (d, J_{C-F}=2.9 Hz), 145.8, 135.0 (d, J_{C-F}=6.3 Hz), 131.2 (d, J_{C-F}=8.7 Hz), 128.5 (d, J_{C-F}=2.6 Hz), 122.8, 119.7 (d, J_{C-F}=26.6 Hz), 105.9 (d, J_{C-F}=22.0 Hz)

HRMS calc'd for C₉H₆BrFN [M+H]⁺: 225.9668 and 227.9647, found: 225.9682 and 225.9659.

TLC: 0.5 R_{f} in 20% EtOAc/Hexanes

5-Bromo-6-fluoro-2-(5,6,7-trifluoro-1H-indol-3-yl)quinoline (20): 5-Bromo-6-fluoroquinoline 19 (2.65 g, 11.70 mmol) and trifluoroindole 11 (1.0 g, 5.85 mmol) were mixed well in a 20 mL microwave vial. Then, 4M HCl solution in 1,4-dioxane (1.76 mL, 7.02 mmol) was added to the vial which resulted in a reddish orange suspension. Reaction vial was sealed and irradiated in a Biotage microwave reactor at 160° C. for 1 h without stirring. Resulted reddish orange brick-like solid was suspended in a mixture of MeOH and DCM (100 mL, 1:1 ratio) using sonication and the slurry was transferred to a separatory funnel. The slurry was dissolved with DCM (200 mL) and washed with saturated NaHCO₃ (150 mL). Organic layer was concentrated to dryness and the crude was purified by flash chromatography (Biotage, 100% DCM). The isolated solid was further purified by trituration with hexanes to give the title compound as off-white solid (1.2 g, 52% yield).

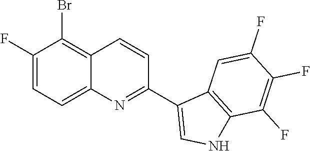 [SMILES: Fc1cc2c(-c3ccc4c(Br)c(F)ccc4n3)c[nH]c2c(F)c1F]

¹H NMR (500 MHz, DMSO-d₆): δ 12.56 (s, 1H), 8.65-8.57 (m, 2H), 8.40 (d, J=8.9 Hz, 1H), 8.26 (d, J=9.0 Hz, 1H), 8.21 (dd, J=9.2, 5.2 Hz, 1H), 7.79 (t, J=8.9 Hz, 1H).

¹⁹F NMR (470 MHz, DMSO-d₆): δ−106.8, −144.3 (d, J_{C-F}=22.2 Hz), −155.2 (d, J_{C-F}=20.5 Hz), −169.2 (t, J_{C-F}=21.5 Hz).

¹³C NMR (150 MHz, DMSO-d₆): δ 155.9 (d, J_{C-F}=244.7 Hz), 154.6 (d, J_{C-F}=2.7 Hz), 146.2 (dd, J_{C-F}=237.0, 11.4 Hz), 145.1, 138.3-136.4 (m), 136.7-134.7 (m), 133.9 (d, J_{C-F}=5.8 Hz), 130.8 (br-d-like), 130.6 (d, J_{C-F}=8.6 Hz), 125.3 (d, J_{C-F}=2.5 Hz), 121.7 (d, J_{C-F}=9.7 Hz), 121.4 (dd, J_{C-F}=10.5, 5.0 Hz), 121.0, 119.4 (d, J_{C-F}=26.1 Hz), 115.5 (d, J_{C-F}=4.6 Hz), 105.0 (d, J_{C-F}=22.0 Hz), 104.2 (dd, J_{C-F}=20.2, 3.6 Hz).

HRMS calc'd for C₁₇H₈BrF₄N₂ [M+H]⁺: 394.9807 and 396.9787, found: 394.9626 and 396.9637.

TLC: 0.6 R_{f} in 100% DCM

6-Fluoro-2-(5,6,7-trifluoro-1H-indol-3-yl)quinoline-5-carboxylic acid (21). Bromo compound 20 (260 mg, 0.66 mmol) was dissolved in dry ether (25 mL) and cooled down to −78° C. n-Butyl lithium (1.6 M in hexanes, 1.23 mL, 1.97 mmol) was added dropwise and stirring was continued for 30 min at −78° C. Then a slow stream of CO₂ gas was bubbled into the reaction mixture for 30 min. The reaction mixture was poured into crushed dry ice, quenched with slow addition of water (30 mL) and the pH was adjusted to 4 and extracted with EtOAc (50 mL×2). Combined organic layer was dried over Na₂SO₄ and concentrated to dryness under reduced pressure to afford the crude carboxylic acid as pale yellow foam (250 mg). TLC analysis of the crude indicated a mixture of product and unreacted starting material and this material was used as such in the next step without any further purification.

### 6-Fluoro-N-methyl-2-(5,6,7-trifluoro-1H-indol-3-yl)quinoline-5-carboxamide (23a)

Compound 23a was obtained from carboxylic acid 21 (90 mg, 0.25 mmol), DIPEA (0.17 mL, 1.0 mmol), methylamine (2 M in THF, 0.25 mL, 0.50 mmol), HATU (189 mg, 0.50 mmol) and DMF (4 mL) using the procedure as that described to obtain 4. The crude product was the purified by flash chromatography (Biotage, a gradient from 0 to 80% EtOAc/Hexanes) to yield the compound 23a as off-white solid (90 mg, 96% yield).

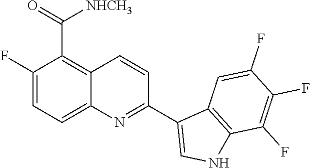 [SMILES: CNC(=O)c1c(F)ccc2nc(-c3c[nH]c4c(F)c(F)c(F)cc34)ccc12]

¹H NMR (600 MHz, DMSO-d₆): δ 12.52 (s, 1H), 8.71 (q, J=4.6 Hz, 1H), 8.65 (ddd, J=11.2, 7.1, 1.6 Hz, 1H), 8.56 (s, 1H), 8.21 (dd, J=9.2, 5.2 Hz, 1H), 8.19-8.12 (m, 2H), 7.68 (t, J=9.1, 1H), 2.89 (d, J=4.6 Hz, 3H).

¹³C NMR (150 MHz, DMSO-d₆): δ 163.3, 155.0 (d, J_{C-F}=245.8 Hz), 154.1 (d, J_{C-F}=2.3 Hz), 146.1 (dd, J_{C-F}=236.8, 11.4 Hz), 144.5, 138.3-136.4 (m), 136.7-134.6 (m), 133.2 (d, J_{C-F}=5.3 Hz), 131.9 (d, J_{C-F}=9.2 Hz), 130.6 (d, J_{C-F}=2.6 Hz), 123.7 (d, J_{C-F}=6.1 Hz), 121.7 (d, J_{C-F}=9.5 Hz), 121.5 (dd, J_{C-F}=10.3, 5.0 Hz), 120.6 (d, J_{C-F}=19.6 Hz), 120.2, 119.2 (d, J_{C-F}=26.2 Hz), 115.8 (d, J_{C-F}=4.6 Hz), 104.2 (dd, J_{C-F}=20.2, 3.6 Hz), 26.1.

HRMS calc'd for C₁₉H₁₂F₄N₃O [M+H]⁺: 374.0916, found: 374.0922.

TLC: 0.4 R_{f} in 75% EtOAc/Hexanes

### 6-Fluoro-N-isopropyl-2-(5,6,7-trifluoro-1H-indol-3-yl)quinoline-5-carboxamide (23b)

Compound 23b was obtained from carboxylic acid 21 (160 mg, 0.44 mmol), DIPEA (0.31 mL, 1.78 mmol), isopropylamine (75 μL, 0.89 mmol), HATU (337 mg, 0.89 mmol) and DMF (6 mL) using the procedure as that described to obtain 4. The crude product was the purified by flash chromatography (Biotage, a gradient from 0 to 80% EtOAc/Hexanes) to yield the compound 23b as off-white solid (115 mg, 62% yield).

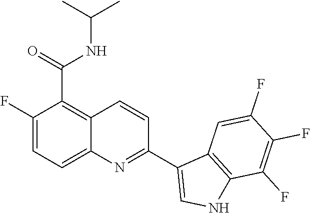 [SMILES: CC(C)NC(=O)c1c(F)ccc2nc(-c3c[nH]c4c(F)c(F)c(F)cc34)ccc12]

¹H NMR (600 MHz, DMSO-d₆): δ 12.51 (s, 1H), 8.70 (d, J=7.7 Hz, 1H), 8.65 (ddd, J=10 11.3, 7.1, 1.6 Hz, 1H), 8.55 (s, 1H), 8.21 (dd, J=9.2, 5.3 Hz, 1H), 8.18 (d, J=8.9 Hz, 1H), 8.14 (d, J=8.9 Hz, 1H), 7.67 (t, J=9.0, 1H), 4.26-4.15 (m, 1H), 1.22 (d, J=6.6 Hz, 6H).

¹³C NMR (150 MHz, DMSO-d₆): δ 161.8, 154.9 (d, J_{C-F}=245.6 Hz), 154.1 (d, J_{C-F}=2.3 Hz), 146.1 (dd, J_{C-F}=236.8, 11.6 Hz), 144.3, 138.3-136.5 (m), 136.5-134.6 (m), 132.9 (d, J_{C-F}=5.2 Hz), 131.6 (d, J_{C-F}=9.2 Hz), 130.5, 123.7 (d, J_{C-F}=6.3 Hz), 121.7 (d, J_{C-F}=9.8 Hz), 121.5 (dd, J_{C-F}=10.6, 5.0 Hz), 120.9 (d, J_{C-F}=19.8 Hz), 120.3, 119.2 (d, J_{C-F}=26.1 Hz), 115.9 (d, J_{C-F}=5.1 Hz), 104.2 (dd, J_{C-F}=20.0, 3.6 Hz), 41.2, 22.3 (2C).

HRMS calc'd for C₂₁H₁₆F₄N₃O [M+H]⁺: 402.1229, found: 402.1220.

TLC: 0.5 R_{f} in 75% EtOAc/Hexanes

6-Fluoro-N,N-dimethyl-2-(5,6,7-trifluoro-1H-indol-3-yl)quinoline-5-carboxamide (23c): Compound 23c was obtained from carboxylic acid 21 (160 mg, 0.44 mmol), DIPEA (0.31 mL, 1.78 mmol), dimethylamine (2 M in THF, 0.44 mL, 0.89 mmol), HATU (337 mg, 0.89 mmol) and DMF (5 mL) using the procedure as that described to obtain 4. The crude product was the purified by flash chromatography (Biotage, a gradient from 0 to 100% EtOAc/Hexanes) to yield the compound 23c as off-white solid (149 mg, 87% yield).

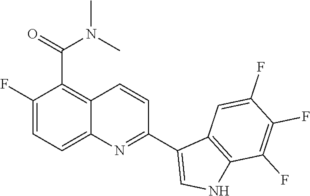 [SMILES: CN(C)C(=O)c1c(F)ccc2nc(-c3c[nH]c4c(F)c(F)c(F)cc34)ccc12]

¹H NMR (600 MHz, DMSO-d₆): δ 12.52 (s, 1H), 8.64 (dd, J=11.2, 7.1 Hz, 1H), 8.57 (d, J=2.3 Hz, 1H), 8.23 (dd, J=9.2, 5.3 Hz, 1H), 8.16 (d, J=8.9 Hz, 1H), 7.99 (d, J=9.0 Hz, 1H), 7.73-7.67 (m, 1H), 3.16 (s, 3H), 2.83 (s, 3H).

¹³C NMR (150 MHz, DMSO-d₆): δ 163.7, 154.3 (d, J_{C-F}=2.3 Hz), 154.1 (d, J_{C-F}=244.7 Hz), 146.1 (dd, J_{C-F}=236.8, 11.4 Hz), 144.5, 138.3-136.5 (m), 136.4-134.7 (m), 132.9 (d, J_{C-F}=5.3 Hz), 131.8 (d, J_{C-F}=9.1 Hz), 130.6 (d, J_{C-F}=2.7 Hz), 123.0 (d, J_{C-F}=6.6 Hz), 121.8-121.6 (m), 121.45 (dd, J_{C-F}=10.6, 5.0 Hz), 120.5, 119.3 (d, J_{C-F}=20.4 Hz), 119.1 (d, J_{C-F}=25.6 Hz), 115.8 (d, J_{C-F}=4.6 Hz), 104.2 (dd, J_{C-F}=20.2, 3.6 Hz), 37.5, 34.2.

HRMS calc'd for C₂₀H₁₄F₄N₃O [M+H]⁺: 388.1073, found: 388.1067; calc'd for C₂₀H₁₃F₄N₃NaO [M+Na]⁺: 410.0892, found: 410.0879.

TLC: 0.5 R_{f} in 75% EtOAc/Hexanes

N-Cyclopropyl-6-fluoro-2-(5,6,7-trifluoro-1H-indol-3-yl)quinoline-5-carboxamide (23d (13795)): Compound 23d was obtained from carboxylic acid 21 (110 mg, 0.28 mmol), DIPEA (0.19 mL, 1.11 mmol), cyclopropylamine (40 μL, 0.56 mmol), HATU (212 mg, 0.56 mmol) and DMF (4 mL) using the procedure as that described to obtain 4. The crude product was the purified by flash chromatography (Biotage, a gradient from 0 to 100% EtOAc/Hexanes). The isolated solid was re-purified by another flash chromatography (Biotage, isocratic 60% EtOAc/Hexanes) followed by trituration with DCM (3 mL) to yield the compound 23d as off-white solid (16 mg, 14% yield).

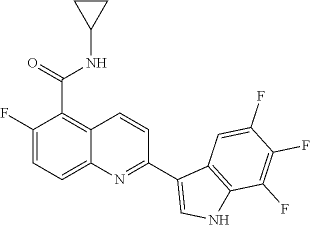 [SMILES: O=C(NC1CC1)c1c(F)ccc2nc(-c3c[nH]c4c(F)c(F)c(F)cc34)ccc12]

¹H NMR (600 MHz, DMSO-d₆): δ 12.52 (s, 1H), 8.85 (d, J=4.4 Hz, 1H), 8.64 (dd, J=11.2, 6.9 Hz, 1H), 8.55 (s, 1H), 8.21 (dd, J=9.2, 5.2 Hz, 1H), 8.17 (d, J=9.0 Hz, 1H), 8.14 (d, J=9.0 Hz, 1H), 7.67 (t, J=9.1, 1H), 3.01-2.94 (m, 1H), 0.81-0.73 (m, 2H), 0.61-0.54 (m, 2H).

¹³C NMR (150 MHz, DMSO-d₆): δ 163.9, 155.0 (d, J_{C-F}=245.8 Hz), 154.1, 146.1 (dd, J_{C-F}=236.7, 11.3 Hz), 144.3, 138.3-136.4 (m), 136.7-134.6 (m), 133.0 (d, J_{C-F}=5.3 Hz), 131.8 (d, J_{C-F}=9.1 Hz), 130.5 (d, J_{C-F}=2.6 Hz), 123.7 (d, J_{C-F}=6.1 Hz), 121.7 (d, J_{C-F}=9.4 Hz), 121.4 (dd, J_{C-F}=10.7, 5.1 Hz), 120.5 (d, J_{C-F}=19.7 Hz), 120.3, 119.1 (d, J_{C-F}=26.1 Hz), 115.8 (d, J_{C-F}=4.7 Hz), 104.2 (dd, J_{C-F}=20.1, 3.6 Hz), 22.8, 5.8 (2C).

HRMS calc'd for C₂₁H₁₄F₄N₃O [M+H]⁺: 400.1073, found: 400.1080.

TLC: 0.5 R_{f} in 75% EtOAc/Hexanes

5-(6-Fluoro-2-(5,6,7-trifluoro-1H-indol-3-yl)quinolin-5-yl)thiazole (22a): In a microwave vial, thiazole (180 μL, 2.53 mmol) and Pd(PPh₃)₄ (30 mg, 0.025 mmol) were added to the solution of bromo compound 20 (200 mg, 0.51 mmol) and KOAc (150 mg, 1.52 mmol) in DMAc (2 mL). The microwave vial was then sealed and irradiated in a Biotage microwave reactor at 150° C. for 2 h. Reaction mixture was diluted with EtOAc (30 mL) and washed with water (15 mL). Organic layer was concentrated to dryness and purified by flash chromatography (Biotage, a gradient from 0-50% EtOAc/Hexanes) to yield compound 22a as off-white solid (130 mg, 64% yield).

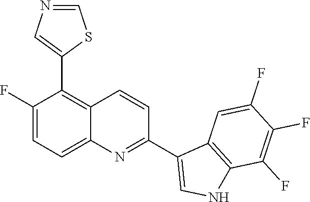 [SMILES: Fc1cc2c(-c3ccc4c(-c5cncs5)c(F)ccc4n3)c[nH]c2c(F)c1F]

¹H NMR (600 MHz, DMSO-d₆): δ 12.51 (s, 1H), 9.42 (d, J=0.8 Hz, 1H), 8.65 (ddd, J=11.3, 7.1, 1.6 Hz, 1H), 8.54 (s, 1H), 8.29 (dd, J=9.3, 5.2 Hz, 1H), 8.15 (d, J=0.8 Hz, 1H), 8.14 (d, J=9.3 Hz, 1H), 8.08 (dd, J=8.9, 0.8 Hz, 1H), 7.78 (t, J=9.3 Hz, 1H).

¹³C NMR (150 MHz, DMSO-d₆): δ 156.5 (d, J_{C-F}=246.6 Hz), 156.4, 154.1 (d, J_{C-F}=2.4 Hz), 146.1 (dd, J_{C-F}=236.8, 11.3 Hz), 144.7, 144.6, 138.3-136.4 (m), 136.6-134.6 (m), 132.8 (d, J_{C-F}=5.7 Hz), 132.2 (d, J_{C-F}=9.4 Hz), 130.5 (d, J_{C-F}=2.6 Hz), 126.2, 125.7 (d, J_{C-F}=3.9 Hz), 121.8-121.6 (m), 121.5 (dd, J_{C-F}=10.5, 5.0 Hz), 120.4, 119.1 (d, J_{C-F}=26.4 Hz), 115.7 (d, J_{C-F}=4.6 Hz), 112.8 (d, J_{C-F}=16.1 Hz), 104.2 (dd, J_{C-F}=20.3, 3.6 Hz).

HRMS calc'd for C₂₀H₁₀F₄N₃S [M+H]⁺: 400.0532, found: 400.0544.

TLC: 0.2 R_{f} in 50% EtOAc/Hexanes

5-(6-Fluoro-2-(5,6,7-trifluoro-1H-indol-3-yl)quinolin-5-yl)oxazole (22b): In a microwave vial, oxazole (166 μL, 2.53 mmol) and Pd(PPh₃)₄ (30 mg, 0.025 mmol) were added to the solution of bromo compound 20 (200 mg, 0.51 mmol) and KOAc (150 mg, 1.52 mmol) in DMAc (2 mL). The microwave vial was then sealed and irradiated in a Biotage microwave reactor at 150° C. for 2 h. Reaction mixture was diluted with EtOAc (30 mL) and washed with water (15 mL). Organic layer was concentrated to dryness and purified by flash chromatography (Biotage, a gradient from 0-50% EtOAc/Hexanes) to yield compound 22b as off-white solid (90 mg, 46% yield).

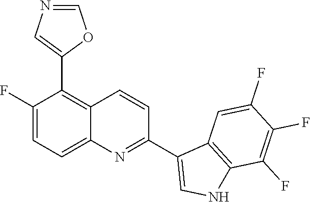 [SMILES: Fc1cc2c(-c3ccc4c(-c5cnco5)c(F)ccc4n3)c[nH]c2c(F)c1F]

¹H NMR (600 MHz, DMSO-d₆): δ 12.52 (s, 1H), 8.71 (s, 1H), 8.64 (dd, J=11.2, 7.0 Hz, 1H), 8.57 (d, J=2.5 Hz, 1H), 8.42 (d, J=9.0 Hz, 1H), 8.31-8.25 (m, 1H), 8.17 (d, J=9.1 Hz, 1H), 7.79 (t, J=9.6 Hz, 1H), 7.69 (d, J=1.7 Hz, 1H).

¹³C NMR (150 MHz, DMSO-d₆): δ 156.5 (d, J_{C-F}=249.8 Hz), 154.2 (d, J_{C-F}=2.2 Hz), 153.1, 146.2 (dd, J_{C-F}=236.8, 11.3 Hz), 144.7, 142.5, 138.3-136.4 (m), 136.7-134.7 (m), 133.1 (d, J_{C-F}=5.6 Hz), 132.8 (d, J_{C-F}=9.5 Hz), 130.6, 128.0 (d, J_{C-F}=5.9 Hz), 124.0 (d, J_{C-F}=3.9 Hz), 121.7 (d, J_{C-F}=9.3 Hz), 121.5 (dd, J_{C-F}=10.5, 5.0 Hz), 120.5, 119.3 (d, J_{C-F}=26.1 Hz), 115.7 (d, J_{C-F}=4.7 Hz), 110.0 (d, J_{C-F}=14.4 Hz), 104.2 (dd, J_{C-F}=19.9, 3.8 Hz).

HRMS calc'd for C₂₀H₁₀F₄N₃O [M+H]⁺: 384.0760, found: 384.0772.

TLC: 0.2 R_{f} in 50% EtOAc/Hexanes

Di-tert-butyl ester derivative (24)

NaH (190 mg, 60% suspension in oil, 4.7 mmol) was suspended in 10 mL of dry DMF at RT. The parent drug 4 (1.12 g, 2.9 mmol) was suspended in 3 mL of DMF, and added to the reaction mixture as a slurry, and rinsed over with 1 mL×2 DMF. Reaction mixture was stirred at RT for 10 min, at which point the solids had mostly dissolved. Catalytic amount of NaI (50 mg, 0.3 mmol) was added as solid, followed by the addition of chloromethyl phosphate reagent (neat, 850 μL, 3.3 mmol). After stirring at RT overnight, the reaction mixture was dumped into a stirred NH₄H₂PO₄—NH₃ buffer (1.5 M×50 mL), and rinsed over with DCM. The mixture was extracted with DCM (100 mL in 3 portions), washed once with NH₄Cl, and the aqueous was back extracted with DCM (20 mL). The combined organic was concentrated to dryness by rotary evaporation. The residue was triturated in MTBE (˜20 mL), and the mixture was aged at RT for 0.5 h. The mixture was filtered, and the filter cake was washed with 5 mL MTBE. The solid was dried under high vacuum for 1 h at RT to give the product as a pale pinkish-brown powder (1.36 g, 76.7% yield).

¹H NMR (500 MHz, DMSO-d₆): δ 8.78 (dd, J=11.0, 7.0 Hz, 1H), 8.64 (s, 1H), 8.61 (d, J=9.0 Hz, 1H), 8.51 (d, J=7.8 Hz, 1H), 8.23 (d, J=8.4 Hz, 1H), 8.09 (d, J=9.0 Hz, 1H), 7.78 (dd, J=8.4, 7.1 Hz, 1H), 7.63 (dd, J=7.0, 1.1 Hz, 1H), 6.12 (d, J_{H-P}=11.7 Hz, 2H), 4.28-4.12 (m, 1H), 1.33 (s, 18H), 1.23 (d, J=6.6 Hz, 6H).

¹⁹F NMR (470 MHz, DMSO-d₆): δ−142.36 (d, J=21.7 Hz, F₅), −156.21 (d, J=20.5 Hz, F₇), −167.78 (t, J=21.5 Hz, F₆).

HRMS calc'd for C₃₀H₃₆F₃N₃O₅P [M+H]⁺: 606.2339, found: 606.2342.

TLC: 0.2 R_{f} in 60% EtOAc/Hex

### Methylene Phosphate Prodrug (5)

The di-tert-butyl ester derivative 13 (1.33 g, 2.20 mmol) was suspended in a mixture of 7 mL DCM and 13 mL toluene in a 50 mL RBF cooled in a tap water bath. TFA (1.1 mL, 6.5 eq.) was added over 2 min. The resulting clear solution was stirred at RT for 6 h, at which point the reaction mixture had turned into a suspension. The mixture was added to a stirred mixture of MeOH (20 mL), water (30 mL), sat. Na₂CO₃ (30 mL), and EtOAc (50 mL). A few drops of 1% phenylphthalein in ethanol was added as pH indicator. Additional solid Na₂CO₃ was added as needed to keep the system basic. The solids slowly dissolved and the aqueous was visually uniformly turbid. Layers were separated and the organic layer was back-extracted with half-saturated Na₂CO₃. The aqueous layers were combined and purified on 120 g C18 in multiple 50-100 mL portions using a gradient of unbuffered MeOH-water. Pure fractions were concentrated to remove most of MeOH. The remaining aqueous solution, ˜200 mL, was passed through a 20 mL Amberlite IR120-Na ion exchange resin column, eluted with water, to remove any ammonium cation. The eluted liquid was concentrated to ˜20 mL, filtered through 0.45 um PTFE syringe filter, rinsed over with more water (10 mL×2). The filtrate was lyophilized overnight to give the title compound 5 as a white powder (1.09 g, 92% Yield).

¹H NMR (500 MHz, D₂O): δ 8.37 (dd, J=9.0, 0.8 Hz, 1H), 8.16 (s, 1H), 8.16 (dd, J=10.7, 7.1 Hz, 1H), 8.11 (d, J=8.5 Hz, 1H), 7.80 (d, J=9.0 Hz, 1H), 7.80 (dd, J=8.5, 7.1 Hz, 1H), 7.67 (dd, J=7.1, 1.2 Hz, 1H), 5.82 (d, J_{H-P}=7.1 Hz, 2H), 4.28 (m, 1H), 1.34 (d, J=6.6 Hz, 3H).

¹³ C NMR (150 MHz, D₂O): δ 170.13, 154.38, 147.28, 146.79, 138.53 (m), 136.9 (m), 134.15, 133.52, 133.33, 129.92, 129.36, 125.21, 123.11, 122.29 (m), 121.16 (m), 120.67, 116.22 (m), 103.42 (m), 72.62, 42.63, 21.47 (2C).

¹⁹F NMR (375 MHz, D₂O): δ−143.49 (d, J=21.2 Hz, F₅), −156.65 (d, J=20.2 Hz, F₇), −167.65 (t, J=20.5 Hz, F₆).

³¹P NMR (160 MHz, D₂O): δ 2.08 (s).

HRMS calc'd for C₂₂H₂₀F₃N₃O₅P [M+H]⁺: 494.1087, found: 494.1091, calc'd for C₂₂H₁₉F₃N₃NaO₅P [M+Na]⁺: 516.0907, found: 516.0911.

### General Procedure for Compounds

In microwave vessel, quinoline (2.0 eq.) and indole (1.0 eq.) were mixed together and the system was purged with N₂/vacuum (3 times) and sealed. Then, HCl solution 4 M in 1,4-dioxane (1.2 eq.) was added using syringe (exothermic reaction). The reaction mixture was irradiated in a microwave reactor at 160° C. for 2 h. The reaction mixture was taken up with a minimum of MeOH and poured in to a mixture of EtOAc and saturated NaHCO₃ solution. The resulting biphasic solution was extracted. The aqueous phase was separated and extracted with EtOAc twice. The organic layers were combined, washed with 0.01 M critic acid solution, saturated NaHCO₃ solution, dried over Na₂SO₄ and concentrated to dryness under reduced pressure. Finally, the crude was purified by automated combi-flash to afford the pure compound.

### 2-(7-Methyl-1H-indol-3-yl)quinoline

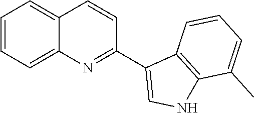 [SMILES: Cc1cccc2c(-c3ccc4ccccc4n3)c[nH]c12]

¹H NMR (500 MHz, DMSO-d₆): δ (ppm)=11.63 (s, 1H), 8.72 (d, J=7.9 Hz, 1H), 8.35 (d, J=2.8 Hz, 1H), 8.25 (d, J=8.7 Hz, 1H), 8.09 (d, J=8.7 Hz, 1H), 8.03 (d, J=8.3 Hz, 1H), 7.88 (d, J=7.8 Hz, 1H), 7.74-7.68 (m, 1H), 7.48 (t, J=7.4 Hz, 1H), 7.12 (t, J=7.5 Hz, 1H), 7.02 (d, J=7.0 Hz, 1H), 2.54 (s, 3H).

¹³C NMR (125 MHz, DMSO-d₆): δ (ppm)=156.14, 148.27, 137.19, 135.98, 129.77, 128.86, 128.08, 128.02, 126.28, 125.82, 125.31, 123.10, 121.27, 120.98, 120.70, 119.79, 116.41, 17.27.

HRMS calc'd for C₁₈H₁₅N₂ [M+H]⁺: 259.1230, found: 259.1229

### 2-(5-Methyl-1H-indol-3-yl)quinoline

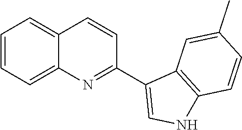 [SMILES: Cc1ccc2[nH]cc(-c3ccc4ccccc4n3)c2c1]

¹H NMR (400 MHz, DMSO-d₆): δ 12.64 (s, 1H), 8.97 (s, 1H), 8.89 (d, J=8.1 Hz, 1H), 8.58 (d, J=8.2 Hz, 1H), 8.42 (d, J=8.9 Hz, 1H), 8.22 (d, J=8.0 Hz, 1H), 8.10 (s, 1H), 8.02 (t, J=7.6 Hz, 1H), 7.77 (t, J=7.5 Hz, 1H), 7.52 (d, J=8.3 Hz, 1H), 7.18 (dd, J=8.3, 0.9 Hz, 1H), 2.51 (s, 3H).

HRMS calc'd for C₁₈H₁₅N₂ [M+H]⁺: 259.1230, found: 259.1218

### 2-(7-Ethyl-1H-indol-3-yl)quinoline

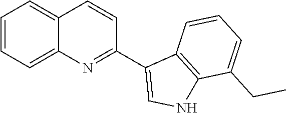 [SMILES: CCc1cccc2c(-c3ccc4ccccc4n3)c[nH]c12]

¹H NMR (500 MHz, DMSO-d₆): δ 11.63 (s, 1H), 8.73 (d, J=7.9 Hz, 1H), 8.34 (d, J=2.9 Hz), 8.25 (d, J=8.7 Hz, 1H), 8.09 (d, J=8.7 Hz, 1H), 8.03 (d, J=8.4 Hz, 1H), 7.88 (d, J=7.2 Hz, 1H), 7.71 (ddd, J=8.3, 7.0, 1.4 Hz, 1H), 7.48 (ddd, J=7.5, 6.5, 1.0 Hz, 1H), 7.15 (t, J=7.8 Hz, 1H), 7.05 (d, J=6.9 Hz, 1H), 2.93 (q, J=7.5 Hz, 2H), 1.32 (t, J=7.5 Hz, 3H).

¹³C NMR (125 MHz, DMSO-d₆): δ 156.14, 148.27, 136.38, 135.97, 129.76, 128.86, 128.07, 127.98, 127.62, 126.28, 126.00, 125.30, 121.28, 121.08, 120.74, 119.80, 116.39, 24.09, 15.01.

HRMS calc'd for C₁₉H₁₇N₂ [M+H]⁺: 273.1386, found: 273.1389

### 2-(7-Bromo-1H-indol-3-yl)quinoline

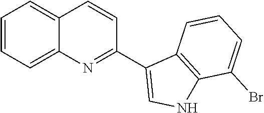 [SMILES: Brc1cccc2c(-c3ccc4ccccc4n3)c[nH]c12]

¹H NMR (600 MHz, DMSO-d₆): δ 11.87 (s, 1H), 8.94 (d, J=7.9 Hz, 1H), 8.42 (d, J=2.8 Hz, 1H), 8.29 (d, J=8.6 Hz, 1H), 8.13 (d, J=8.7 Hz, 1H), 8.06 (d, J=8.4 Hz, 1H), 7.91 (d, J=8.0 Hz, 1H), 7.73 (ddd, J=8.4, 7.2, 1.2 Hz, 1H), 7.51 (ddd, J=7.8, 6.6, 0.6 Hz, 1H), 7.45 (dd, J=7.5, 0.5 Hz, 1H), 7.17 (t, J=7.8 Hz, 1H).

¹³C NMR (150 MHz, DMSO-d₆): δ 155.00, 147.65, 135.81, 135.51, 129.43, 128.78, 128.46, 127.65, 127.36, 125.96, 125.19, 124.73, 122.17, 121.76, 119.31, 116.67, 104.34.

HRMS calc'd for C₁₇H₁₂BrN₂ [M+H]⁺: 323.0178 and 325.0159, found: 323.0172 and 325.0153

### 2-(7-Fluoro-1H-indol-3-yl)quinoline

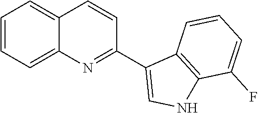 [SMILES: Fc1cccc2c(-c3ccc4ccccc4n3)c[nH]c12]

¹H NMR (500 MHz, DMSO-d₆): δ 12.17 (s, 1H), 8.71 (d, J=8.0 Hz, 1H), 8.44 (d, J=2.8 Hz, 1H), 8.29 (d, J=8.7 Hz, 1H), 8.10 (d, J=8.7 Hz, 1H), 8.05 (d, J=8.3 Hz, 1H), 7.91 (d, J=7.5 Hz, 1H), 7.73 (t, J=7.5 Hz, 1H), 7.51 (t, J=7.5 Hz, 1H), 7.21-7.15 (m, 1H), 7.06 (dd, J=11.4, 7.8 Hz, 1H).

¹³C NMR (125 MHz, DMSO-d₆): δ 155.50, 149.63 (d, J_{C-F}=243.4 Hz), 148.13, 136.29, 129.92, 129.85 (d, J_{C-F}=5.2 Hz), 129.18, 128.90, 128.14, 126.41, 125.63, 125.52 (d, J_{C-F}=13.2 Hz), 121.14 (d, J_{C-F}=6.2 Hz), 119.76, 119.30 (d, J_{C-F}=3.0 Hz), 116.97 (d, J_{C-F}=1.3 Hz), 107.43 (d, J_{C-F}=15.6 Hz).

HRMS calc'd for C₁₇H₁₂FN₂ [M+H]⁺: 263.0979, found: 263.0976

### 5-Bromo-2-(7-methyl-1H-indol-3-yl)quinoline

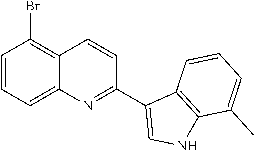 [SMILES: Cc1cccc2c(-c3ccc4c(Br)cccc4n3)c[nH]c12]

¹H NMR (400 MHz, DMSO-d₆): δ 11.74 (s, 1H), 8.69 (d, J=8.0 Hz, 1H), 8.42 (d, J=3.0 Hz, 1H), 8.36 (dd, J=9.0, 0.7 Hz, 1H), 8.23 (d, J=9.0 Hz, 1H), 8.06 (dt, J=8.4, 0.9 Hz, 1H), 7.81 (dd, J=7.5, 1.0 Hz, 1H), 7.64 (dd, J=8.4, 7.6 Hz, 1H), 7.13 (t, J=7.6 Hz, 1H), 7.03 (d, J=7.0 Hz, 1H), 2.54 (s, 3H).

¹³C NMR (100 MHz, DMSO-d₆): δ 156.97, 149.20, 137.24, 134.47, 130.54, 129.10, 129.01, 128.88, 125.74, 125.10, 123.32, 121.45, 121.43, 121.37, 121.24, 120.60, 115.71, 17.25.

HRMS calc'd for C₁₈H₁₄BrN₂ [M+H]⁺: 337.0335 and 339.0316, found: 337.0345 and 339.0331.

### 8-Bromo-2-(7-methyl-1H-indol-3-yl)quinoline

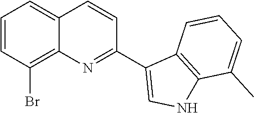 [SMILES: Cc1cccc2c(-c3ccc4cccc(Br)c4n3)c[nH]c12]

¹H NMR (400 MHz, DMSO-d₆): δ 11.72 (s, 1H), 9.04 (d, J=8.0 Hz, 1H), 8.47 (d, J=3.0 Hz, 1H), 8.30 (d, J=8.8 Hz, 1H), 8.20 (d, J=8.7 Hz, 1H), 8.09 (dd, J=7.5, 1.3 Hz, 1H), 7.92 (dd, J=8.1, 1.2 Hz, 1H), 7.39 (t, J=7.8 Hz, 1H), 7.14 (t, J=7.4 Hz, 1H), 7.03 (d, J=7.0 Hz, 1H), 2.54 (s, 3H).

¹³C NMR (100 MHz, DMSO-d₆): δ 156.88, 144.96, 137.23, 136.61, 133.25, 128.92, 128.20, 127.68, 125.91, 125.77, 123.97, 123.36, 121.38, 121.33, 121.23, 120.45, 116.31, 17.25.

HRMS calc'd for C₁₈H₁₄BrN₂ [M+H]⁺: 337.0335 and 339.0316, found: 337.0341 and 339.0324.

### 2-(5,6,7-Trifluoro-1H-indol-3-yl)quinoline

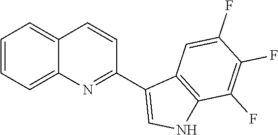 [SMILES: Fc1cc2c(-c3ccc4ccccc4n3)c[nH]c2c(F)c1F]

¹H NMR (600 MHz, CDCl₃): δ 9.99 (s, 1H), 8.21 (d, J=8.4 Hz, 1H), 8.12 (d, J=8.1 Hz, 1H), 7.87 (d, J=8.4 Hz, 1H), 7.84 (d, J=8.4 Hz, 1H), 7.77 (t, J=7.5 Hz, 1H), 7.57 (t, J=7.3 Hz, 1H), 7.23 (ddd, J=9.7, 6.5, 1.6 Hz, 1H), 7.12 (s, 1H).

¹³C NMR (150 MHz, CDCl₃): δ 148.30, 147.32, 146.70 (dd, J_{C-F}=240.3, 12.2 Hz), 138.72, 137.53 (ddd, J_{C-F}=249.3, 13.6, 4.5 Hz), 136.93 (ddd, J_{C-F}=244.8, 17.1, 11.3 Hz), 136.35, 129.70, 128.58, 127.22, 127.06, 126.14, 124.01 (dd, J_{C-F}=6.9, 5.5 Hz), 121.44 (dd, J_{C-F}=7.7, 2.9 Hz), 117.52, 101.92 (m).

HRMS calc'd for C₁₇H₁₀F₃N₂ [M+H]⁺: 299.0791, found: 299.0791.

### General Procedure for Compounds

Halogenated-compound (1.0 eq.), boronic acid (or boronate) (1.1 eq.) and Pd(PPh₃)₄ (10 mol %) was added in microwave vessel (10-20 mL). The vial was sealed with a cap and the system was purged with vacuum and placed under N₂. Then, the solids were dissolved in a mixture of Toluene/EtOH (4.5: 2.0, 0.11 M) and purged with vacuum/N₂ one more time. Then, a solution of K₂CO₃ (3.0 eq) in H₂O (1.8 M) was added. The reaction mixture was stirred and heated overnight at 95° C. with oil bath. The reaction mixture was filtered on plug of celite. The filtrate was poured into water and was extracted with AcOEt three times. The organic layers were combined, washed with brine, dried over Na₂SO₄ and evaporated under reduced pressure. The residue was taken up in DCM/TFA (5:5) for 2 h at rt. The solvents were evaporated under reduced pressure. The residue was then neutralized with saturated NaHCO₃ solution and extracted with AcOEt (×2). The organic layers were washed, dried and concentrated in vacuo. The residue was triturated in DCM or purified by automated combi-flash to afford the pure compound.

### 3-(7-Methyl-1H-indol-3-yl)quinoline

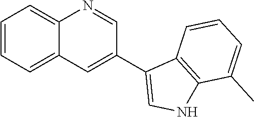 [SMILES: Cc1cccc2c(-c3cnc4ccccc4c3)c[nH]c12]

¹H NMR (400 MHz, DMSO-d₆): δ 11.59 (s, 1H), 9.34 (d, J=2.3 Hz, 1H), 8.62 (d, J=2.1 Hz, 1H), 8.07 (m, 2H), 8.01 (d, J=8.4 Hz, 1H), 7.92 (d, J=7.9 Hz, 1H), 7.69 (td, J=8.4, 1.5 Hz, 1H), 7.61 (td, J=8.1, 1.2 Hz, 1H), 7.12 (t, J=7.6 Hz, 1H), 7.03 (d, J=7.0 Hz, 1H), 2.54 (s, 3H).

¹³C NMR (100 MHz, DMSO-d₆): δ 150.68, 146.10, 136.96, 130.41, 129.74, 129.08, 128.72, 128.64, 128.37, 127.19, 125.16, 125.01, 122.86, 121.80, 120.77, 117.24, 113.06, 17.30.

HRMS calc'd for C₁₈H₁₅N₂ [M+H]⁺: 259.1230, found: 259.1230

### 7-(7-Methyl-1H-indol-3-yl)quinoline

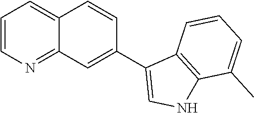 [SMILES: Cc1cccc2c(-c3ccc4cccnc4c3)c[nH]c12]

¹H NMR (600 MHz, DMSO-d₆): δ 11.54 (s, 1H), 8.89 (dd, J=4.2, 1.7 Hz, 1H), 8.34 (dd, J=8.2, 0.9 Hz, 1H), 8.30 (s, 1H), 8.04-8.00 (m, 2H), 7.98 (d, J=2.4 Hz, 1H), 7.87 (d, J=8.0 Hz, 1H), 7.47 (dd, J=8.2, 4.2 Hz, 1H), 7.10 (t, J=7.5 Hz, 1H), 7.02 (d, J=7.0 Hz, 1H), 2.54 (s, 3H).

¹³C NMR (100 MHz, DMSO-d₆): δ 150.65, 148.56, 137.35, 136.61, 135.55, 128.24, 126.41, 125.92, 124.71, 124.62, 124.05, 122.23, 121.36, 120.40, 120.28, 116.64, 115.27, 16.83.

HRMS calc'd for C₁₈H₁₅N₂ [M+H]⁺: 259.1230, found: 259.1228

### 2-(7-Methyl-1H-indol-3-yl)benzo[d]thiazole

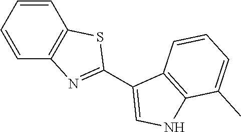 [SMILES: Cc1cccc2c(-c3nc4ccccc4s3)c[nH]c12]

¹H NMR (600 MHz, DMSO-d₆): δ 11.95 (s, 1H), 8.25 (d, J=2.9 Hz, 1H), 8.22 (d, J=7.9 Hz, 1H), 8.06 (d, J=7.9 Hz, 1H), 7.97 (d, J=8.1 Hz, 1H), 7.49 (t, J=7.6 Hz, 1H), 7.37 (t, J=7.6 Hz, 1H), 7.17 (t, J=7.5 Hz, 1H), 7.07 (d, J=7.1 Hz, 1H), 2.54 (s, 3H).

¹³C NMR (150 MHz, DMSO-d₆): δ 162.90, 153.68, 136.26, 133.03, 128.52, 126.09, 124.32, 124.17, 123.25, 121.71, 121.63, 121.53, 121.30, 118.22, 110.81, 16.74.

HRMS calc'd for C₁₆H₁₃N₂S [M+H]⁺: 265.0794, found: 265.0790

### 4-Bromo-2-(7-methyl-1H-indol-3-yl)thiazole

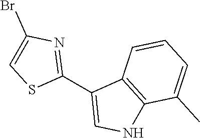 [SMILES: Cc1cccc2c(-c3nc(Br)cs3)c[nH]c12]

¹H NMR (600 MHz, DMSO-d₆): δ 11.86 (s, 1H), 8.16 (s, 1H), 7.93 (d, J=7.7 Hz, 1H), 7.62 (s, 1H), 7.13 (t, J=7.3 Hz, 1H), 7.04 (d, J=6.6 Hz, 1H), 2.52 (s, 3H).

¹³C NMR (151 MHz, DMSO-d₆): δ 164.43, 136.06, 126.82, 123.71, 123.69, 123.09, 121.67, 121.19, 117.45, 113.69, 110.13, 16.71.

HRMS calc'd for C₁₂H₁₀BrN₂S [M+H]⁺: 292.9743 and 294.9722, found: 292.9743 and 294.9723

A person of skill in the art based on the general knowledge in the art and the information provided herein would be able to synthesize the compounds described herein or modify the compounds described herein.

Cell Culture: LNCaP and PC3 human prostate cancer cells were obtained from American Type Culture Collection (ATCC, Manassas, VA) and grown in RPMI 1640 medium supplemented with 5% fetal bovine serum (FBS) (Invitrogen™). The LNCaP eGFP cell line was stably transfected with an androgen-responsive probasin-derived promoter fused to an eGFP reporter (LN-ARR2PB-eGFP) using a lentiviral approach, and were grown in phenol-red-free RPMI 1640 supplemented with 5% CSS. In-house developed MDV3100-resistant LNCaP cells were cultured in RPMI 1640 supplemented with 5% FBS and 10 μM MDV3100. All cells were maintained at 37° C. in 5% CO2.

eGFP Cellular AR Transcription Assay: The AR transcriptional activity was assayed as previously described (Tavassoli, Snoek et al. 2007). LNCaP cells were incubated with compound 13789 for 3 days and fluorescence was measured.

Prostate-Specific Antigen (PSA) Assay: The evaluation of PSA levels secreted into the media was performed in parallel to the eGFP assay using the same plates. After cells were incubated with compound 13789, 13789 prodrug, or 13789 N-methyl for 3 days, 150 μl of the media was taken from each well, and added to 150 μl of PBS. PSA levels were then evaluated using Cobase 411™ analyzer instrument (Roche Diagnostics™) according to the manufacturer's instructions.

Cell Viability Assay: The PC3, LNCaP, and MDV3100-resistant cells were plated at 3,000 cells per well in RPMI 164o containing 5% charcoal stripped serum (CSS) in a 96-well plate, treated with 0.1 nM R1881 and compounds (0-25 μM) for 96 hrs. After 3 days of treatment, cell density was measured using the Prestoblue™ assay (Thermo Fisher Scientific™).

Androgen Displacement Assay: The androgen displacement was assessed with the Polar Screen Androgen Receptor Competitor Green Assay Kit™ as per the instructions of the manufacturer (Lack, Axerio-Cilies et al. 2011).

Assessment of Prostate Surface Antigen and Tumour Growth for Castration-resistant LNCaP Xenografts: 6-8-Week-old nude mice (Harlan Sprague-Dawley) weighing 25-31 g were subcutaneously inoculated with LNCaP cells (106 cells in BD Matrigel, BD Biosciences) at the posterior dorsal site. Tumor volume, body weight, and serum PSA levels were measured weekly. Mice were castrated when serum PSA levels reached 50 ng/ml. When PSA recovered to 25 ng/ml, mice were randomized into three oral treatment groups as follows: vehicle, enzalutamide (MDV), or 13789 prodrug. Calipers were used to measure the three perpendicular axes of each tumor to calculate the tumor volume.

### High Performance Liquid Chromatography (HPLC)

HPLC analyses were conducted on Agilent 1100 HPLC with the a G1322A vacuum degasser; G1312A binary pump; G1367A autosampler; G1316A column compartment; and G1321A fluorescence detector, using the full-range fluorescence scans on compounds 13566 and 13822 (prodrug of 13789) were performed to determine the best excitation and emission wave lengths (i.e. excitation at 230 nm and emission at 450 nm) suitable for the detection of both compounds. A Halo5 C18 column was used (3.0×50 mm, 5 μm particle size; injection volume: 1 μL) having a mobile phase A: 10% acetonitrile, 90% water, 10 mM NH₄HCO₃ and a mobile phase B: 80% acetonitrile, 20% water, 10 mM NH₄HCO₃, with a flow rate: 0.7 mL/min.

### Calibration and Validation

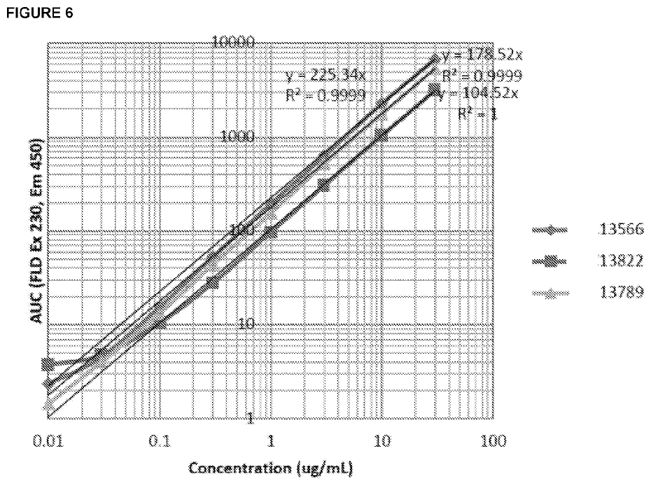

Standard samples of 13566, 13789, and 13822 were prepared by serial dilutions from 100 μg/mL stock solutions in 50% acetonitrile. Nine levels with half-log spacing were prepared (100, 30, 10, 3, 1, 0.3, 0.1, 0.03, and 0.01 μg/mL) to construct the calibration curve (see FIG. 6). Area under curve (AUC) values vs. concentrations were plotted in Excel and a linear fit was conducted. A constraint to cross the curve at (0,0) were applied. All compounds achieved 0.03 μg/mL LOQ sensitivity (0.01 μg/mL LOD), and good linearity between 0.03 and 30 μg/mL (R>0.999). At 100 μg/mL detector saturation was observed.

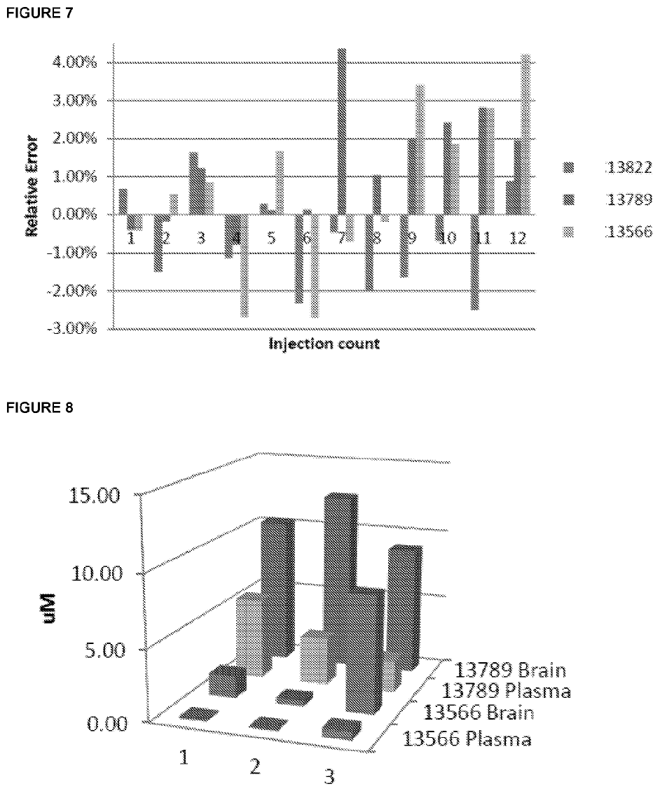

To probe the method precision, QC blocks consisting two blanks and one injection of a 3-component mixture at 0.3 μg/mL were inserted throughout the calibration curve and sample analysis sequence, and the results of repeated injections were plotted (as percent differences from mean values). The result gave standard error of 2.2%, 1.5%, and 1.4% for 13566, 13789, and 13822, respectively (see FIG. 7).

### Sample Formulation, Dosing, and Sample Collection

13822 was water soluble and was formulated as plain aqueous solution at 3.75 mg/mL. 13566 was not soluble in water at the required concentration and although a 4:1 mixture of propylene glycol and poly(ethylene glycol)-400 had been used in the past as the dosing vehicle, there was concern that when the 100% organic solution hit the aqueous content in the GI tract, uncontrolled precipitation would occur that can cause unpredictable variations in drug uptake and other PK parameters. A formulation screen was conducted to use various pharmaceutically acceptable surfactants and additives to develop a low organic solution or metastable heterogeneous suspension suitable for oral dosing.

Finally, a formulation consisted of 12.5% PEG400, 1.25% Poloxamer-188, and 0.38% methyl cellulose was found to give a milk-like uniform micro-suspension at 3.75 mg/mL that was stable for at least 24 h. The dosing solutions were given to mice (C57 Black 6) by oral gavage at 8 μL per gram of body weight (rounded to the nearest 10 μL), corresponding to 30 mg/kg. After 3 h, the animals were euthanized, and then blood and brain samples were collected and stored at 4° C. until analysis.

### Sample Preparation and Analysis

Blood samples (clotted) were centrifuged at 9,000 RPM for 2 min×2. Aliquots of the serum (30 μL) were placed in 150 μL micro-centrifuge tubes and acetonitrile (30 μL) were added. The mixture was vortexed briefly and allowed to stand at room temperature for 30 min. Full brains were weighed into 2 mL micro-centrifuge tubes and equal amount of acetonitrile (w/v) was added. The mixture was homogenized with an Omni Tissue Master 125 homogenizer and the mixtures were allowed to stand at room temperature for 30 min. The samples were centrifuged at 9000 RPM for 2 min×2. The supernatants were transferred to HPLC sample vials with conical inserts, and then analyzed by HPLC.

TABLE 1
HPLC analysis results of blood and brain samples

| | Blood | | | Brain | | | Brain/ |
|---|---|---|---|---|---|---|---|
| Group/ | | μg/ | | | μg/ | | Blood |
| Animal | AUC | mL | μM | AUC | mL | μM | Ratio |
| 13566/M1 | 0.028 | 0.11 | 0.110 | 45.82 | 0.41 | 1.57 | 14.3 |
| 13566/M2 | 0.019 | 0.07 | 0.073 | 11.59 | 0.10 | 0.40 | 5.4 |
| 13566/M3 | 0.157 | 0.61 | 0.606 | 237.45 | 2.11 | 8.16 | 13.5 |
| 13822/M1** | 194.03 | 2.17 | 5.67 | 354.96 | 3.98 | 10.37 | 1.8 |
| 13822/M2 | 114.14 | 1.28 | 3.34 | 429.33 | 4.81 | 12.55 | 3.8 |
| 13822/M3 | 76.82 | 0.86 | 2.24 | 308.62 | 3.46 | 9.02 | 4.0 |

*The dilution factor (2) has been taken into consideration when calculating the drug concentrations
**Plasma sample failed to inject due to trapped air bubbles in the vial insert; sample was re-analyzed five days later, at which point partial evaporation of the solvent likely had happened and the concentration would be unreliable

### Mice Testing

All compounds were dosed orally to mice, and after 3 hours, the animals were euthanized and blood and brain samples were collected. Drug levels were analyzed by HPLC equipped with a fluorescence detector as described herein.

### Example 1—In Silico Modelling for Potential BF3 Binders

Using a previously described (Axerio-Cilies, P. et al. Inhibitors of Androgen Receptor Activation Function-2 (AF2) Site Indentified Through Virtual Screening. J Med Chem 2011 54(18):6197-205), consensus-based in silico methodology we conducted a virtual screen of ˜10 million purchasable chemical substances from the ZINC database to identify BF3-specific binders (also one NCI compound). The screening method used a combination of large-scale docking, ligand-based QSAR modeling, pharmacophore search, molecular field analysis, molecular-mechanic and molecular dynamic simulations (Cherkasov, A. et al. Progressive docking: a hybrid QSAR/docking approach for accelerating in silico high throughput screening. J Med Chem. 2006, 49, 7466-7478; Cherkasov, A. et al. ‘Inductive’ charges on atoms in proteins: comparative docking with the extended steroid benchmark set and discovery of a novel SHBG ligand. J Chem Inf Model 2005, 45, 1842-1853; and Santos-Filho, O. A. and Cherkasov, A. Using molecular docking, 3D-QSAR, and cluster analysis for screening structurally diverse data sets of pharmacological interest. J Chem Inf Model 2008, 48, 2054-2065). The results from each stage of this multi-parametric approach were compiled and the compounds were ranked using a consensus scoring procedure. The highest ranked compounds were visualized and initial candidates, predicted to have a high potential for binding to the BF3 pocket, were selected for empirical testing.

### Example 2—Cell-Based Testing

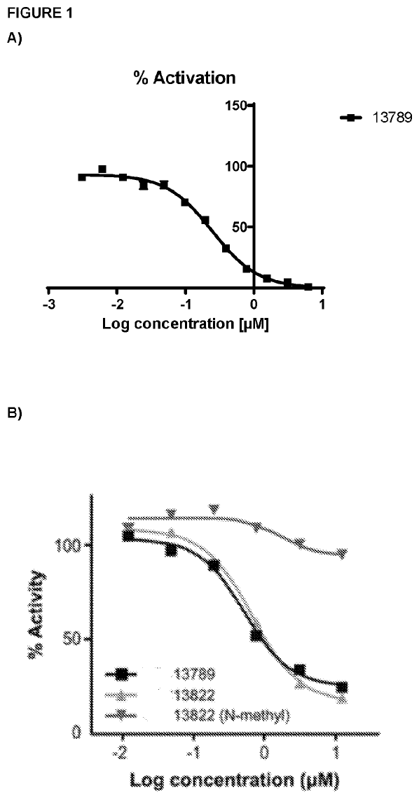

Compounds were tested for their ability to inhibit AR-related transcription events. Using an eGFP expression assay, compound 13789 inhibits AR transcriptional activity (FIG. 1A). In this assay, the expression of eGFP is under the control of an androgen-responsive probasin-derived promoter and can be used to quantify AR transcriptional activity. Compound 13789 and 13789 prodrug suppress AR-mediated PSA expression in a concentration dependant manner using LNCaP cells (FIG. 1B). The 13789 N-Methyl form is a closely related derivative to 13789 prodrug that is insensitive to phosphatases and acts as a negative control in the assay.

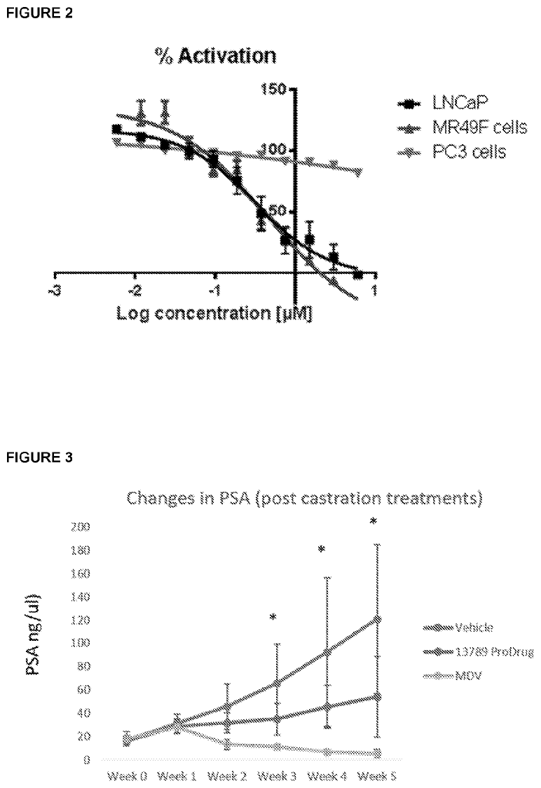

Based on the inhibitory activity of these compounds toward AR transcription and PSA expression, the ability of compound 13789 to inhibit the proliferation of AR-dependent human PCa cells was further evaluated. LNCaP, PC3 and MR49F human PCa cells were serum-starved in RPMI 1640 media (Invitrogen™) supplemented with 5% charcoal-stripped serum (CSS) (RPMI 1640 medium with 5% CSS) for 5 days prior to transfection. Cells were then treated with compound 13789 at various concentrations and 0.1 nM R1881 (in 100% ethanol) for 72 h and read using Prestoblue™ assay. Following hormone activation of the AR (0.1 nM R1881), compound 13789 elicited a concentration-dependent inhibition of growth in AR-dependent LNCaP cells (FIG. 2). A similar potency for cell-growth inhibition was achieved when compound 13789 was evaluated using the Enzalutamide-resistant cell line, MR49F, but not with AR-independent PC3 cells.

The ability of the compound 13789 to displace DHT from the androgen binding site (ABS) of recombinant wild type LBD was measured using a fluorescent polarization assay (PolarScreen™, Life Technologies™) at various concentrations (TABLE 2). Very little displacement was observed confirming that this compound does not have a mechanism of action through the ABS.

TABLE 2
DHT Displacement from the ABS site
of recombinant wild type LBD

| CPDS | % Disp. @ 1 uM | % Disp. @ 10 UM |
|---|---|---|
| Casodex | 75 ± 1.1 | 100 ± 2.3 |
| Enzalutamide | 33 ± 0.4 | 90 ± 0.8 |
| 13789 | 4 ± 3.8 | 11 ± 3.1 |

Even at high concentrations of 13789 (10 μM) no significant ABS inhibition was observed (Table 2). Overall these results demonstrate that this BF3 inhibitor is a potent AR antagonist.

The binding pose of 13566 at the BF3-binding site (FIG. 12) was determined using AR co-crystal structure (4HLW with 2.5 Å resolution) and the ICM docking program. The predicted 13566 binding pose suggests that there was a limited volume to accommodate large substitutions around the 5′, 6′ and 7′ positions of the indole; therefore the H atoms of positions 5′, 6′ and 7′ were replaced with halogens hoping to improve the stability of the indole fragment and retain the AR-BF3 inhibition activity (Table 3). Small substitutions such as —F at 5′, 6′ and 7′ positions led to improved activity with the corresponding IC₅₀ established between 0.01 and 0.23 μM (13610, 13642, 13770, 13622, 13624, 13641, 13607, 13618 and 13601). On the other hand, compounds with larger substitutions such as —Cl (13641), or methyl substitution at positions 5′ or 6′ (13627, 13592, 13625 and 13619) appeared weaker or even inactive (Table 3). Furthermore, no significant improvement towards in vitro metabolic stability could be achieved without compromising potency. Thus, more stable analogues 13627 (T1/2=154 min.) and 13592 (T1/2=94 min.) exhibited a significant decrease in AR inhibition potency (1.5 and 1.22 μM respectively), suggesting that or substituent pattern on the indole ring alone could affect the compounds' inhibitory profile, but not biostability.

Structural modifications of 13566 were expanded on its quinoline moiety, where substitutions at 5, 7 and 8 positions resulted in favorable effects on metabolic stability in human microsomes (Table 4). With substitutions such as 5-CF3 and —CONHCH3; 7-F and —Cl; and 8-F, the half-life measured in microsomes approximately doubled (T1/2>53 min) compared to 13566 (T1/2=21 min). This improvement in stability was further improved with 8-Me, —Cl, —Br substitutions (T1/2>97 min).

Based on the above observations, the next batch of derivatives was designed with substitutions on both indole and quinoline moieties. Thus, the 5′, 6′, and 7′ tri-fluoro-substituted indole moiety of 13770 demonstrated comparable potency with 13566 (Table 3), yet appeared more stable. Further enhancements on the quinoline moiety were designed using the 13770 backbone to possibly further improve the metabolic stability. The 5-substituted quinoline analogs of 13770 (13808, 13795, 13719, 13807 and 13789) all demonstrated potent inhibition of AR transcriptional activity (eGFP assay) resulting in IC50 ranging from 0.035 to 0.19 μM and good microsome stability (Table 5). From these, 13789 was identified as the most promising candidate (FIG. 12) with potent AR inhibition and significantly improved microsomal stability (t1/2=206 min). Docking of 13789 at the AR BF3 site revealed that the molecule forms a favorable hydrogen-bonding interaction between its amide fragment and the carbonyl of Asn833 of the pocket (FIG. 12), which could be attributed to strong on-target activity of the substance.

TABLE 3
Effects of various substitution on 5′-, 6′-, 7′- positions of the indole
moiety of 13566 on the AR inhibition activity and microsomal half-life.

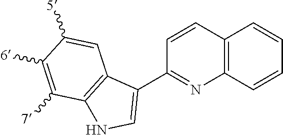

| | | | | IC₅₀-eGFP | T_{1/2} |
|---|---|---|---|---|---|
| Compound | 5′ | 6′ | 7′ | (μM) | (min.) |
| 13610 | F | H | F | 0.03 | 27 |
| 13642 | | F | Cl | 0.03 | 11 |
| 13770 | F | F | F | 0.09 | 15 |
| 13622 | | | Cl | 0.10 | 17 |
| 13624 | F | | Cl | 0.10 | 23 |
| 13641 | Cl | | F | 0.13 | 38 |
| 13607 | | F | Me | 0.17 | 26 |
| 13618 | | F | F | 0.19 | 10 |
| 13601 | F | | Me | 0.23 | 66 |
| 13627 | Cl | | Cl | 1.50 | 154 |
| 13592 | Me | | Me | 1.59 | 94 |
| 13625 | Me | | Cl | 1.90 | 18 |
| 13619 | | Me | F | 2.17 | 15 |

TABLE 4
Effects of various substitution on 5′-, 7′-, 8′- positions of
the quinoline moiety of VPC-13566
on the AR inhibition activity and microsomal half-life.

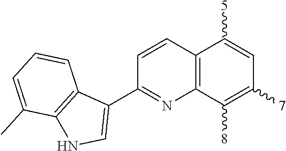

| | | | | IC₅₀-eGFP | T_{1/2} |
|---|---|---|---|---|---|
| Compound | 5 | 7 | 8 | (μM) | (min.) |
| 13566 | H | H | H | 0.21 | 21 |
| 13620 | OH | H | H | inactive | 17 |
| 13623 | F | H | H | inactive | 13 |
| 13602 | CF₃ | H | H | 0.24 | 75 |
| 13597 | CONHCH₃ | H | H | 0.23 | 59 |
| 13611 | H | F | H | 0.30 | 53 |
| 13606 | H | Cl | H | 1.65 | 89 |
| 13595 | H | H | Me | 0.18 | 97 |
| 13692 | H | H | F | 0.10 | 59 |
| 13593 | H | H | Cl | 0.20 | 105 |
| 13677 | H | H | Br | 0.16 | 144 |

TABLE 5
Analogs of VPC-13770 and their IC₅₀-eGFP and microsomal stability T1/2

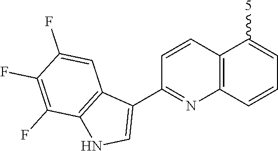

| | | IC₅₀-eGFP | T_{1/2} |
|---|---|---|---|
| Compound | 5′ | (μM) | (min.) |
| 13808 | 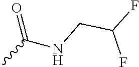 | 0.04 | ND |
| 13795 | 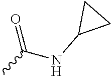 | 0.07 | ND |
| 13793 | 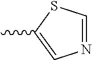 | 0.07 | ND |
| 13719 | 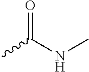 | 0.11 | 70 |
| 13807 | 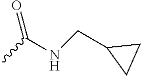 | 0.18 | ND |
| 13789 | 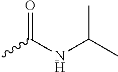 | 0.19 | 206 |
| 13801 | 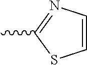 | 0.50 | ND |
| 13761 | 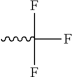 | 0.55 | 67 |
| 13806 | 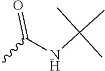 | 0.74 | ND |
| 13804 | 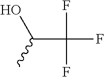 | 32 | ND |

### Example 3—In Vivo Testing

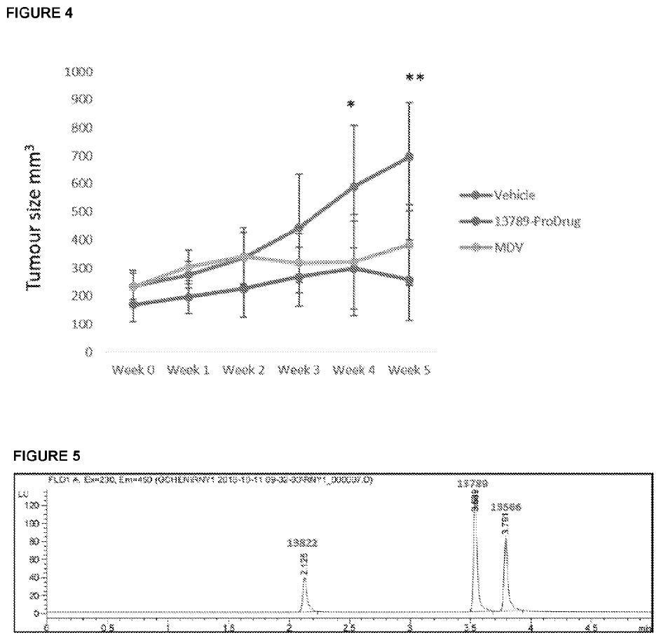

FIGS. 3 and 4 show that 13789 prodrug has favorable therapeutic characteristics in vivo. In addition to reducing serum PSA levels (FIG. 3), treatment with 13789 prodrug inhibited tumour growth to levels comparable with enzalutamide treatment (FIG. 4). Thus, targeting DNA binding by the AR can be as effective in vivo as preventing nuclear translocation by enzalutamide.

### Example 4—Cell-Based Testing of Tri-Substituted Indole Compounds

TABLE 6 shows the eGFP and PSA IC₅₀ values and half-life values for a series of compounds tested and their associated structures with identifiers.

TABLE 6
Cell-based Testing of Tri-substituted Indole Compounds

| | | IC₅₀ eGFP | PSA IC₅₀ | |
|---|---|---|---|---|
| Identifier | Structure | [μM] | [μM] | Half-life |
| 13789 | 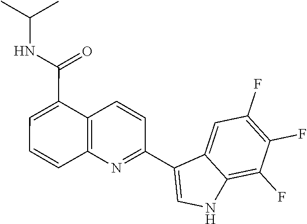 [SMILES: CC(C)NC(=O)c1cccc2nc(-c3c[nH]c4c(F)c(F)c(F)cc34)ccc12] | 0.2 (0.19) | 0.08 | 206 (206.26) |
| 13822 | 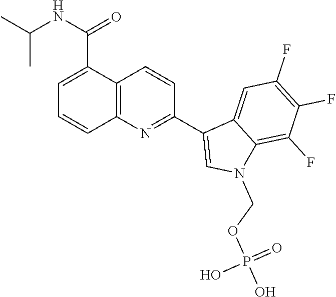 [SMILES: CC(C)NC(=O)c1cccc2nc(-c3cn(COP(=O)(O)O)c4c(F)c(F)c(F)cc34)ccc12] | | | |
| 13795 | 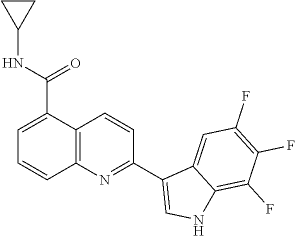 [SMILES: O=C(NC1CC1)c1cccc2nc(-c3c[nH]c4c(F)c(F)c(F)cc34)ccc12] | 0.07 | 0.23 | 261.13 |
| 13797 | 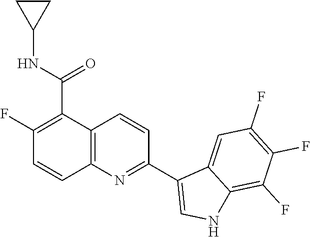 [SMILES: O=C(NC1CC1)c1c(F)ccc2nc(-c3c[nH]c4c(F)c(F)c(F)cc34)ccc12] | 1.98 | 1.5 | ND |
| 13807 | 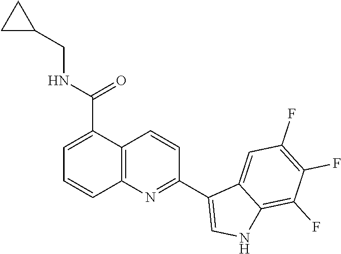 [SMILES: O=C(NCC1CC1)c1cccc2nc(-c3c[nH]c4c(F)c(F)c(F)cc34)ccc12] | 0.18 | 0.16 | ND |
| 13800 | 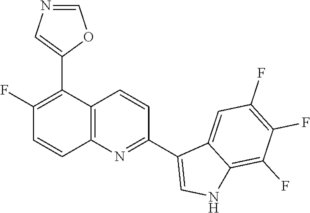 [SMILES: Fc1cc2c(-c3ccc4c(-c5cnco5)c(F)ccc4n3)c[nH]c2c(F)c1F] | | | ND |
| 13794 | 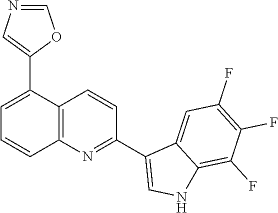 [SMILES: Fc1cc2c(-c3ccc4c(-c5cnco5)cccc4n3)c[nH]c2c(F)c1F] | IC50 > 10 μM | IC50 > 10 μM | ND |
| 13801 | 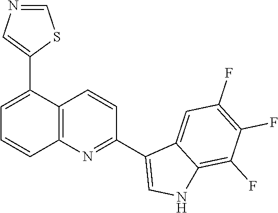 [SMILES: Fc1cc2c(-c3ccc4c(-c5cncs5)cccc4n3)c[nH]c2c(F)c1F] | 0.18 | IC50 > 10 μM | ND |
| 13802 | 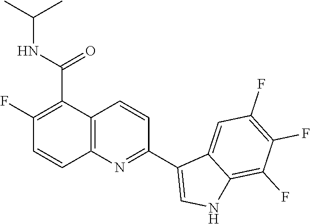 [SMILES: CC(C)NC(=O)c1c(F)ccc2nc(-c3c[nH]c4c(F)c(F)c(F)cc34)ccc12] | IC50 > 10 μM | IC50 > 10 μM | ND |
| 13791 | 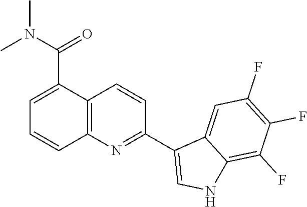 [SMILES: CN(C)C(=O)c1cccc2nc(-c3c[nH]c4c(F)c(F)c(F)cc34)ccc12] | IC50 > 10 μM | IC50 > 10 μM | ND |
| 13803 | 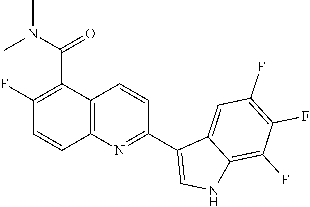 [SMILES: CN(C)C(=O)c1c(F)ccc2nc(-c3c[nH]c4c(F)c(F)c(F)cc34)ccc12] | IC50 > 10 μM | Not tested | ND |
| 13806 | 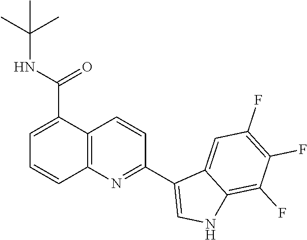 [SMILES: CC(C)(C)NC(=O)c1cccc2nc(-c3c[nH]c4c(F)c(F)c(F)cc34)ccc12] | 0.74 | Not tested | ND |
| 13808 | 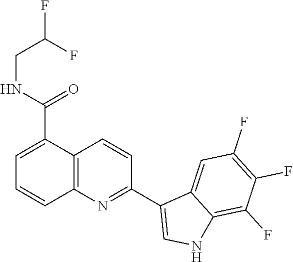 [SMILES: O=C(NCC(F)F)c1cccc2nc(-c3c[nH]c4c(F)c(F)c(F)cc34)ccc12] | 0.04 | | ND |
| 13814 | 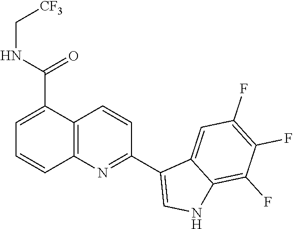 [SMILES: O=C(NCC(F)(F)F)c1cccc2nc(-c3c[nH]c4c(F)c(F)c(F)cc34)ccc12] | | | ND |
| 13813 | 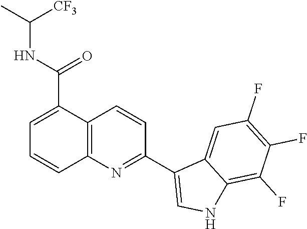 [SMILES: CC(NC(=O)c1cccc2nc(-c3c[nH]c4c(F)c(F)c(F)cc34)ccc12)C(F)(F)F] | | | ND |
| 13793 | 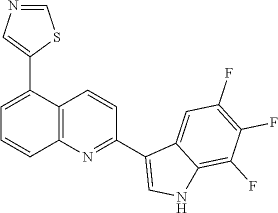 [SMILES: Fc1cc2c(-c3ccc4c(-c5cncs5)cccc4n3)c[nH]c2c(F)c1F] | 0.065 | 0.33 | 104.02 |
| 13799 | 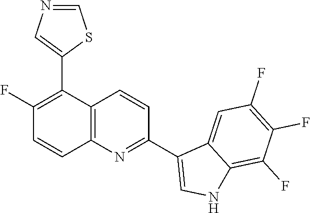 [SMILES: Fc1cc2c(-c3ccc4c(-c5cncs5)c(F)ccc4n3)c[nH]c2c(F)c1F] | 0.56 | 0.75 | ND |
| 13719 | 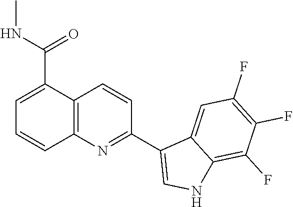 [SMILES: CNC(=O)c1cccc2nc(-c3c[nH]c4c(F)c(F)c(F)cc34)ccc12] | 0.11 | 0.13 | 72.3 |
| 13796 | 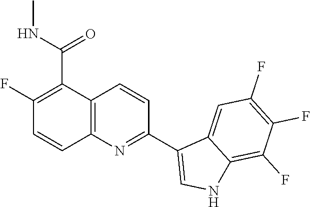 [SMILES: CNC(=O)c1c(F)ccc2nc(-c3c[nH]c4c(F)c(F)c(F)cc34)ccc12] | 5.479 | 0.95 | ND |
| 13761 | 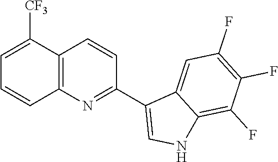 [SMILES: Fc1cc2c(-c3ccc4c(C(F)(F)F)cccc4n3)c[nH]c2c(F)c1F] | 0.5481 | 0.3829 | 66.77 |

### Example 5—In Silico-Based Predictions for Tri-Substituted Indole Compounds

TABLE 7 shows in silico predictions for eGFP and PSA IC₅₀ values and predicted half-life values for a series of compounds not yet tested in cell based assays and their associated structures with identifiers.

TABLE 7
In Silico -based predictions for Tri-substituted Indole Compounds

| | | Probability | Probability | |
|---|---|---|---|---|
| | | of IC₅₀ eGFP ≤ | of PSA IC₅₀ ≤ | Predicted |
| Identifier | Structure | 100 nM | 100 nM | Half-life |
| 16 | 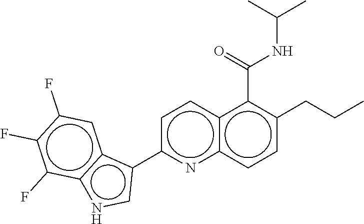 | 0.96865296 | 0.80802625 | 102.38139 |
| 20 | 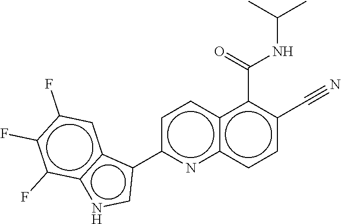 | 0.83548725 | 0.48090386 | 108.08457 |
| 29 | 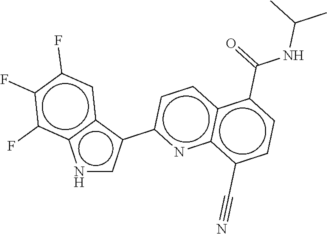 | 0.8218851 | 0.4361342 | 111.53476 |
| 1 | 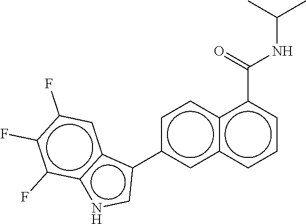 | 0.794252 | 0.89061284 | 52.619255 |
| 21 | 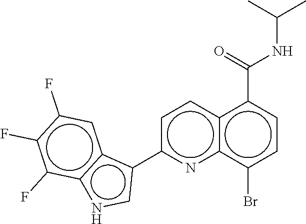 | 0.7933944 | 0.66410923 | 114.58189 |
| 23 | 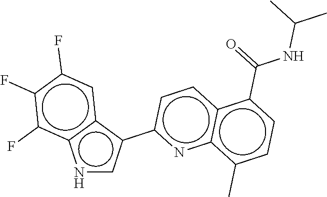 | 0.78051984 | 0.8121626 | 90.26188 |
| 26 | 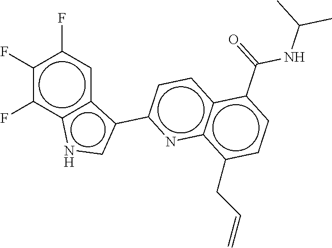 | 0.75747466 | 0.73914516 | 97.88232 |
| 14 | 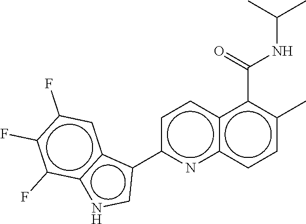 | 0.75101453 | 0.7432456 | 88.28867 |
| 17 | 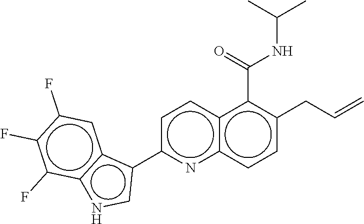 | 0.74024886 | 0.6261453 | 97.692795 |
| 27 | 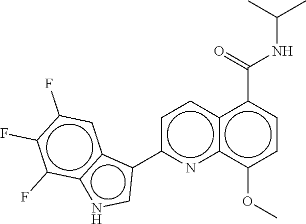 | 0.73744476 | 0.5634492 | 84.75737 |
| 3 | 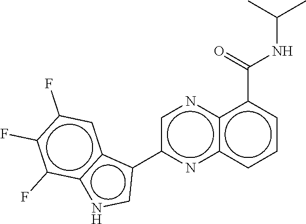 | 0.73655343 | 0.6201855 | 107.10255 |
| 6 | 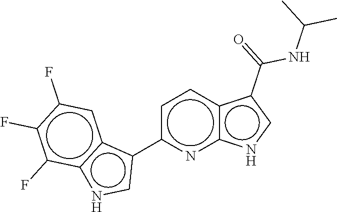 | 0.7171539 | 0.7977825 | 87.24245 |
| 25 | 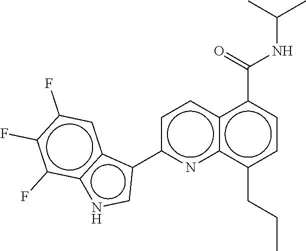 | 0.7087841 | 0.7168357 | 100.04743 |
| 22 | 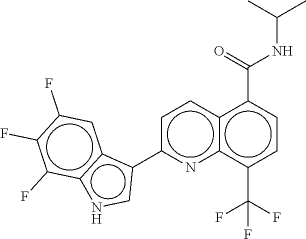 | 0.6739269 | 0.59597635 | 114.10974 |
| 15 | 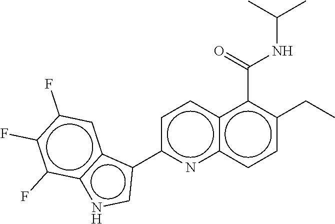 | 0.65619487 | 0.7299891 | 96.06192 |
| 2 | 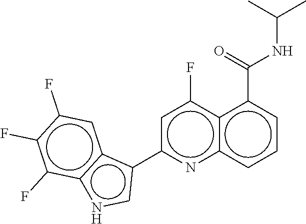 | 0.6444289 | 0.6260849 | 71.81796 |
| 4 | 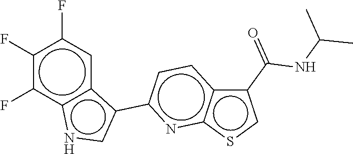 | 0.63058436 | 0.42327526 | 67.6395 |
| 28 | 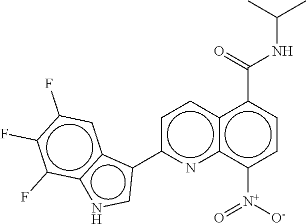 | 0.52370036 | 0.8241459 | 83.45431 |
| 13 | 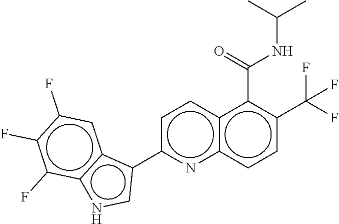 | 0.51333535 | 0.43831307 | 114.78608 |
| 38 | 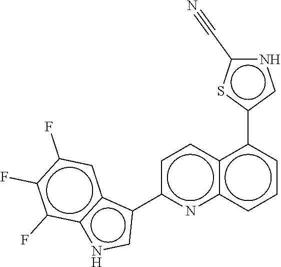 | 0.48128557 | 0.2335806 | 89.51144 |
| 5 | 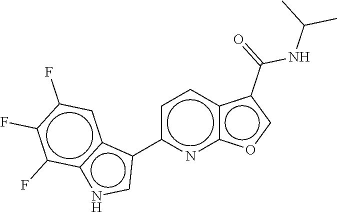 | 0.45798564 | 0.7150696 | 64.35336 |
| 12 | 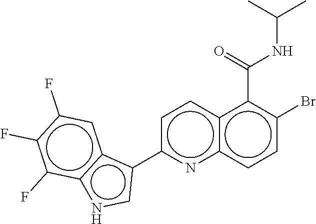 | 0.4553445 | 0.37682232 | 102.54438 |
| 7 | 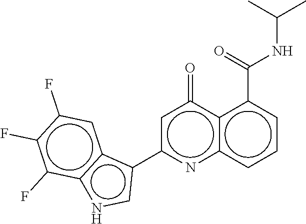 | 0.42580903 | 0.69605815 | 116.79881 |
| 24 | 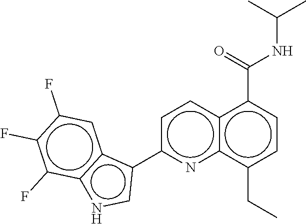 | 0.41952834 | 0.7006095 | 94.19311 |
| 18 | 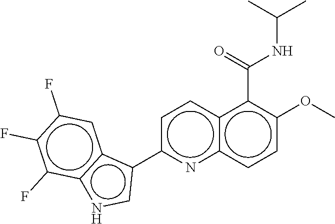 | 0.36890936 | 0.37265715 | 84.3317 |
| 19 | 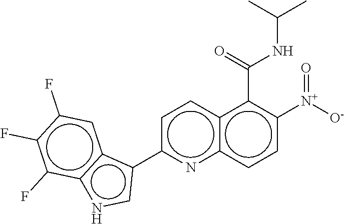 | 0.35139894 | 0.61016613 | 80.30853 |
| 10 | 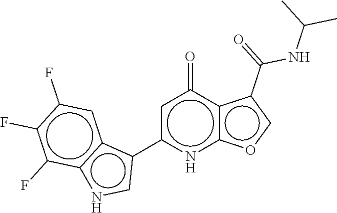 | 0.17063648 | 0.4727874 | 148.70947 |
| 11 | 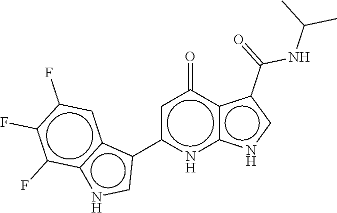 | 0.1396808 | 0.5091994 | 182.05763 |
| 9 | 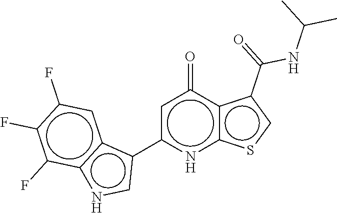 | 0.13099119 | 0.29438242 | 159.3706 |
| 32 | 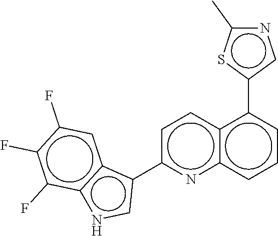 | 0.12873246 | 0.23380616 | 81.86962 |
| 30 | 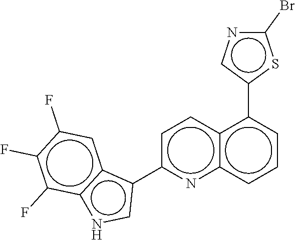 | 0.08408331 | 0.05632016 | 95.73325 |
| 34 | 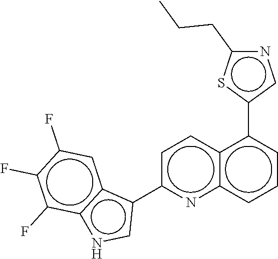 | 0.06470869 | 0.14603078 | 99.39938 |
| 35 | 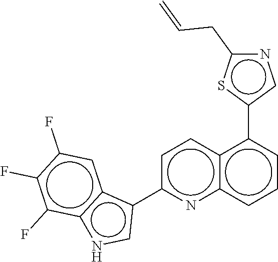 | 0.059941817 | 0.09870722 | 94.806335 |
| 37 | 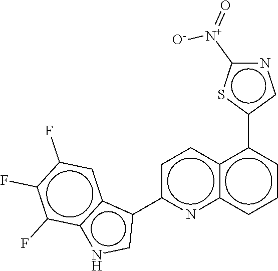 | 0.05596575 | 0.19147971 | 97.16237 |
| 33 | 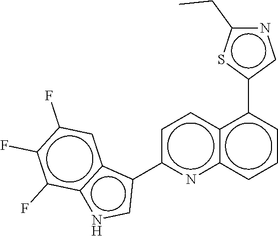 | 0.02373283 | 0.1606717 | 92.37815 |
| 36 | 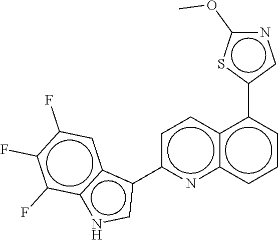 | 0.022648659 | 0.024899796 | 94.27411 |
| 31 | 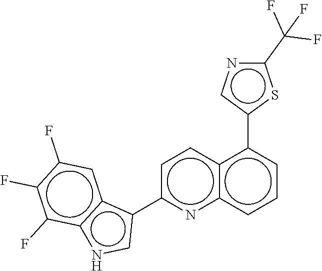 | 0.016029054 | 0.04108787 | 111.55029 |
| 8 | 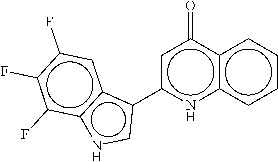 | 0.007128249 | 0.38325062 | 75.85661 |
| 39 | 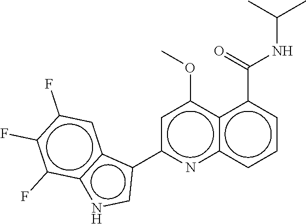 | | | |
| 40 | 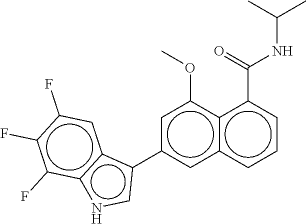 | | | |

### Example 6—13789 Inhibits AR Transcription in Prostate Tumor Cells

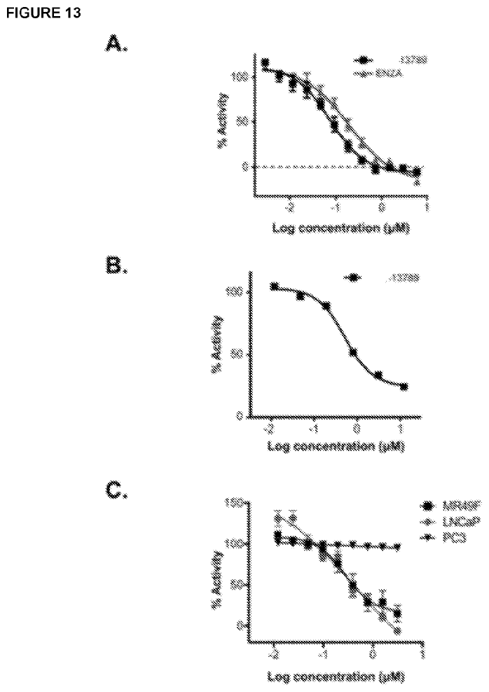

13789 had comparable AR inhibition activity to enzalutamide with an IC₅₀ of 0.19 μM and 0.075 μM, respectively (FIG. 13A) in LNCaP cells using the AR transcriptional green fluorescent protein (eGFP) assay. To further validate 13789 as an AR inhibitor, its effect on the production of endogenous prostate specific antigen (PSA), an androgen-regulated serine protease that is widely used as a biomarker of AR activity in PCa was tested. 13789 inhibited the level of PSA secretion in LNCaP cells with an IC₅₀ of 0.16 μM (FIG. 13B). Next the in vitro activity of 13789 was assessed against androgen-dependent LNCaP, enzalutamide-resistant MR49F cell line, and AR-negative PC3 prostate cancer cells. 13789 treatment caused a significant decrease in the growth of LNCaP cells, with an IC₅₀ of 0.32 μM (FIG. 13C). In contrast, 13789 did not exhibit any inhibitory effect on AR-negative PC3 cells, confirming an AR-directed mechanism of action (FIG. 13C). To validate that this activity occurred via BF3 inhibition rather than androgen displacement at the ABS, the ability of 13789 to displace a fluorescent-tagged androgenic ligand (FluormoneAL Green) from the AR-LBD was measure. Even at high concentrations of 13789 (10 μM) no significant ABS inhibition was observed (Table 4). Overall these results demonstrate that this BF3 inhibitor is a potent AR antagonist. Most importantly, 13789 also demonstrated a great balance of microsomal-stability of T_{1/2}=206 minutes and inhibition activity IC₅₀-eGFP=0.19 μM (FIG. 13A).

### Example 7—13789 Mechanism of Action

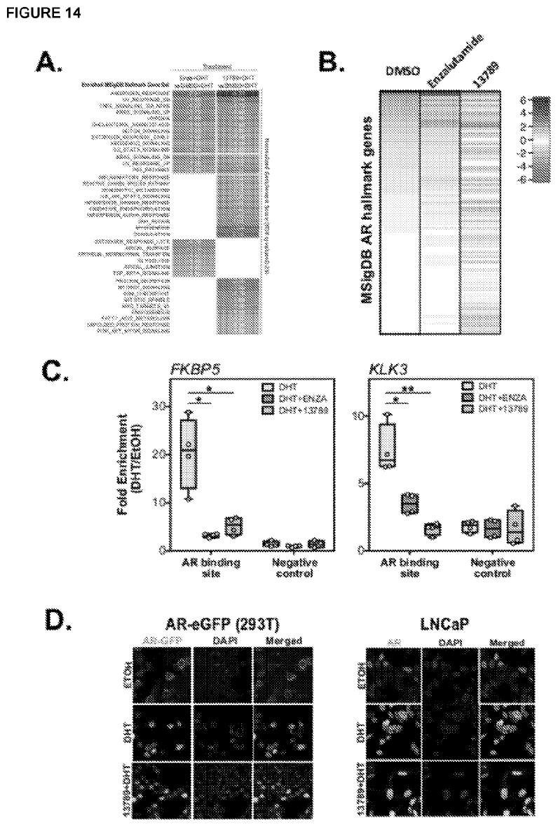

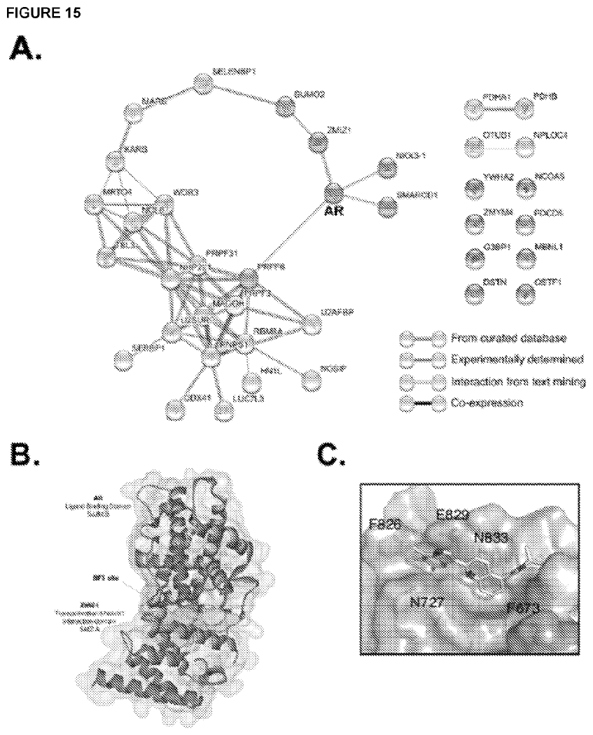

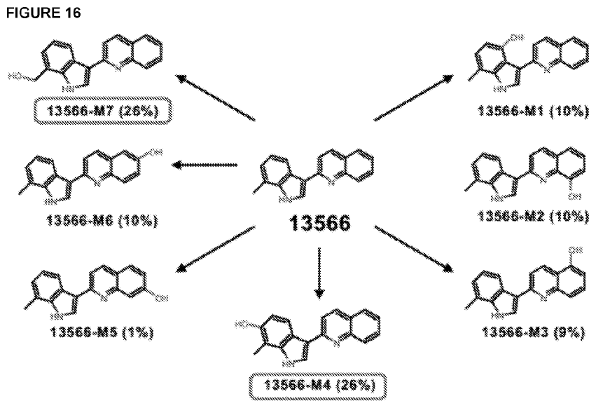

RNA-seq on LNCaP cells was conducted to understand how BF3 inhibition impacted androgen-mediated transcription. Compared to vehicle, 13789 induced differential expression of 1514 genes; 547 upregulated and 967 downregulated. Many of these were unique to 13789 and not found in the enzalutamide treated cells which affected 586 differentially expressed genes (214 upregulated/372 downregulated). Both enzalutamide and 13789 treated cells showed a clear decrease in AR-regulated gene expression, with a greater number of canonical AR genes inhibited by 13789 than enzalutamide as demonstrated by the GSEA normalized enrichment scores (−3.33 vs. −2.31) (FIG. 14A,14B). In addition to the AR pathway, 13789 also caused an upregulation of the p53 pathway which was not observed with enzalutamide. This was not an off-target effect, as this pathway was not affected when cells were treated with 13789 without androgen. It was also found that 13789 significantly reduced the chromatin binding of AR to known enhancers near KLK3 and FKBP5 (FIG. 14C). This was not solely due to impaired translocation, as treatment with 13789 only marginally affected nuclear accumulation of either exogenous (293T) or endogenous (LNCaP) AR (FIG. 14D). Based on the proposed role of BF3 in co-regulator recruitment, we quantified changes to the AR interactome with rapid immunoprecipitation mass spectrometry of endogenous protein (RIME). In DHT treated cells we identified 112 proteins that were significantly enriched compared to EtOH controls including 21 known AR coregulators such as ARID1A, FOXA1 and TRIM24. When treated with 13789, 34 of these interactions were disrupted, including AR-interacting co-regulators (NKX3.1, ZMIZ1/SUMO2), RNA processing (PRPF6) and chromatin remodeling proteins (SMARCD1) (FIG. 14A). As a crystal structure of ZMIZ1 was available, potential AR LBD interaction interfaces was modeled in silico and ranked them by binding affinities. Among ZMIZ1-AR interactions, the highest affinity was found to occur through BF3 (FIG. 15B). The modeled interface area was 742.2 Å² with a solvation free energy (ΔiG) of −6.9 kcal/mol and a ΔiG P-value of 0.274, indicating a specific hydrophobic interaction. Specifically, this interaction occurred via the BF3 residues F826, E830, N834, F674, and N728 which make up the binding pocket of 13789 (FIG. 15C). Taken together, these experimental and computational results demonstrate that inhibition of the AR-BF3 with 13789 prevents co-regulatory recruitment which impairs chromatin binding and androgen-mediated transcription.

### Example 8—Determine Drug Concentration in Mice Brain Tissues

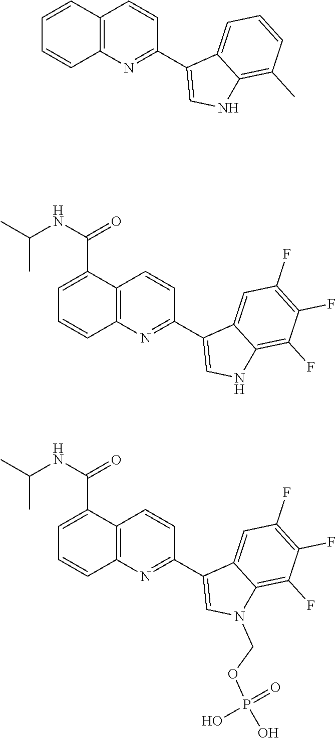

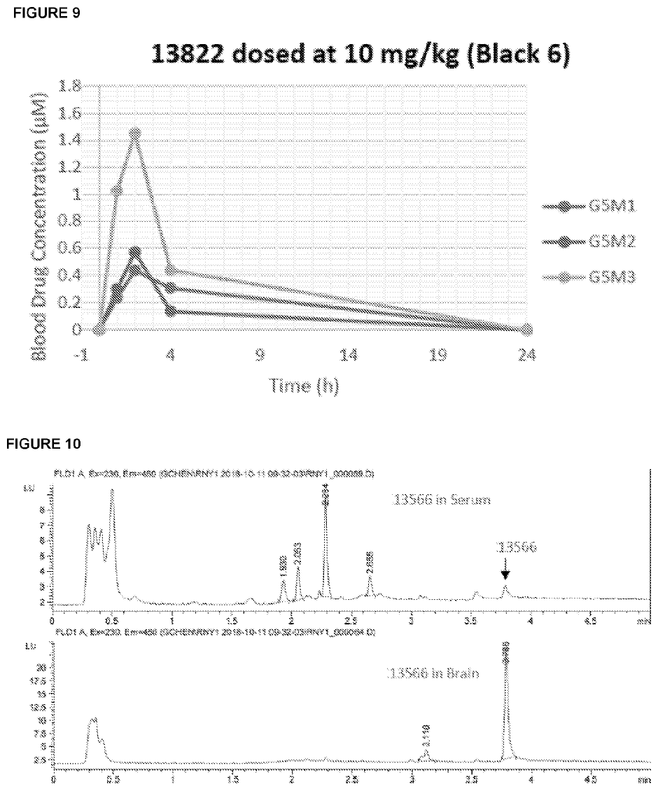

The compounds above were tested for their ability to cross the blood-brain-barrier in mice and to determine the concentrations of the compound in the mouse brain. Under optimized conditions, all three compounds were separated and good peak shapes were achieved (see FIG. 5). Both 13566 and 13789 (dosed as its pro-drug 13822) significantly penetrated the blood-brain-barrier (BBB), and the levels observed in the brain at 3 hour time point were higher than those observed in the blood (see FIG. 8). For 13789, blood drug levels were around 3 μM and brain drug levels were around 10 μM, which gave a brain-to-blood ratio of about three. The observed higher brain drug level could be due to enrichment of 13789 in the brain (preferentially partitioned in the brain due to its higher lipid content), or due to longer elimination half-life in the brain. Compared with a previous study where 13822 was dosed at 10 mg/kg, the observed blood drug level was roughly 3× higher, indicating at this level the drug uptake was likely linear (see FIG. 9).

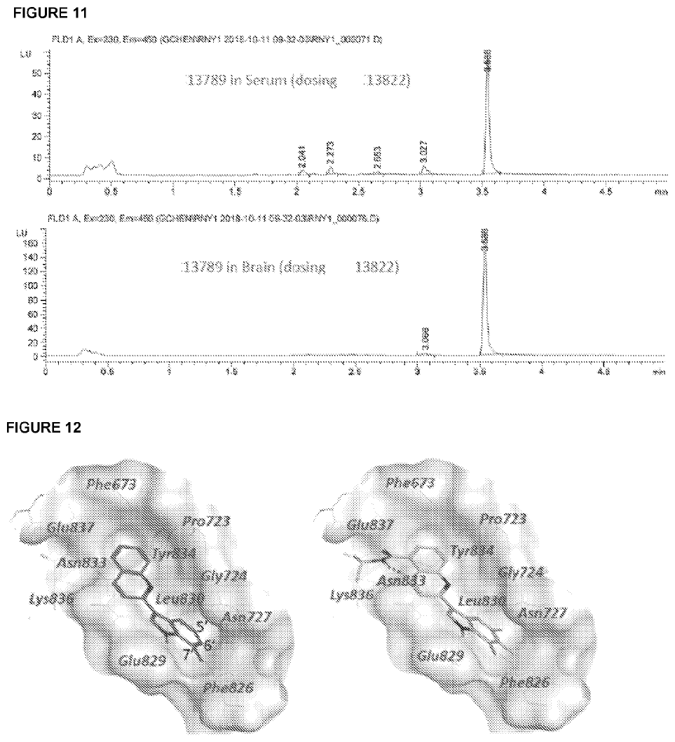

For 13566, the blood concentration level was much lower, and higher variability was observed; at the same time, a few more polar peaks were observed in the chromatogram (peaks before 0.5 min were common for all tissue samples and were likely due to background proteins) as shown in FIG. 10. These were likely due to metabolism of the drug (for example, oxidation, glucuronidation, phosphorylation, etc.). Interestingly, the amount of metabolism seemed to be less in the brain (as judged by the fewer more polar peaks in the chromatogram). This would explain the fact that much higher drug levels (about 10×) were observed in the brain than in the blood. In comparison, the more polar peaks in 13789 samples were far fewer, indicating this compound would be metabolically more stable as shown in FIG. 11.

### Example 9—In Silico-Based Predictions of the Ability to Cross the Blood-Brain-Barrier

TABLE 8 shows in silico predictions of a compounds ability to cross the blood-brain-barrier.

TABLE 8
In Silico -based predictions for Tri-substituted Indole Compounds

| | | | | | Predicted Ability |
|---|---|---|---|---|---|
| | | | | Good | to Cross BBB (0 = |
| Identifier | H logD | XSA | LM/Dhh | Prediction | fail and 1 = pass) |
| 13566 | 3.57 | 53.44 | 5.98 | 1 | 1 |
| 13719 | 3.98 | 42.69 | 5.00 | 1 | 1 |
| 13789 | 4.54 | 62.25 | 7.20 | 0 | 1 |
| 13791 | 4.48 | 53.63 | 5.95 | 1 | 1 |
| 13793 | 4.90 | 59.00 | 7.55 | 0 | 1 |
| 13794 | 4.57 | 46.25 | 6.48 | 0 | 1 |
| 13795 | 4.02 | 58.00 | 6.48 | 0 | 1 |
| 13797 | 4.36 | 59.50 | 6.85 | 0 | 1 |
| 13799 | 5.24 | 62.13 | 7.28 | 0 | 1 |
| 13800 | 4.92 | 48.81 | 6.60 | 0 | 1 |
| 13761 | 5.14 | 54.25 | 4.53 | 1 | 1 |
| 13807 | 4.24 | 68.06 | 6.42 | 0 | 1 |
| 13800 | 4.92 | 48.81 | 6.60 | 0 | 1 |
| 13801 | 4.86 | 66.38 | 8.21 | 0 | 1 |
| 13802 | 4.88 | 63.31 | 7.53 | 0 | 1 |
| 13803 | 4.82 | 56.25 | 6.18 | 0 | 1 |
| 13804 | 4.53 | 70.13 | 6.14 | 0 | 0 |
| 13806 | 4.82 | 67.38 | 9.13 | 0 | 1 |
| 13808 | 4.15 | 58.25 | 6.32 | 0 | 1 |
| 13813 | 4.81 | 78.31 | 7.55 | 0 | 0 |
| 13814 | 4.58 | 66.81 | 6.85 | 0 | 1 |

### Example 10—Pharmacokinetic Properties

TABLE 9

| | | | Pharmacokinetic Parameters | | | | |
|---|---|---|---|---|---|---|---|
| | | | | | | | AUC |
| | Dose | | T_{1/2} | Tₘₐₓ | Cₘₐₓ | Cₘₐₓ | ng*min/ |
| Compound | mg/Kg | Route | (h) | (h) | ng/mL | μM | mL |
| 13789 | 50 | PO | 2.4 | 2 | 1524 | 4.0 | 935991 |
| Sesame oil | | | | | | | |
| 13822 | 100 | PO | 2.9 | 2 | 8278 | 21.6 | 5167228 |
| 0.5% CMC | | | | | | | |

In a proof of concept experiment, steady conversion of the prodrug (13822) to the parent drug (13789) was observed in mouse small intestine tissue homogenate with a half-life of ˜2 hour. Interestingly, the prodrug was effectively saturated at 100 mg/kg in 0.5% CMC, twice the maximum amount that was possible with the parent drug in sesame oil. Oral administration of the phosphate prodrug formulated in 0.5% CMC showed excellent PK levels at 100 mg/kg (Table 9). A Cmax value of 8278 ng/mL was observed that corresponds to the peak concentration of 21.6 μM in plasma which is 5.5 times higher than the 50 mg/kg PO dose of the parent drug formulated in sesame oil.

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims

What is claimed is:

1. A compound of Formula I, 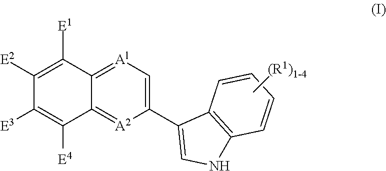
or a pharmaceutically acceptable salt thereof;
wherein
each R¹ is independently selected from the group consisting of halo, C₁₋₆ alkyl, C₁₋₆ haloalkyl, and OC₁₋₆ alkyl;
A¹ is selected from C—OH, C(OCH₃), N, CH, CF, and CCF₃;
A² is selected from N and CH;
E¹ is selected from C(O)NH(C₁₋₆ alkyl), C(O)N(C₁₋₆ alkyl)₂, C(O)NH(C₃₋₆ cycloalkyl), halo, OH, CO₂H, and 5-membered heteroaryl, wherein alkyl and heteroaryl are each optionally substituted with 1, 2, or 3 substituents selected from halo, hydroxy, C₁₋₆ alkyl, C₁₋₆ haloalkyl, C₁₋₆ alkenyl, NO₂, CN, and C₃₋₆ cycloalkyl; and
E², E³, and E⁴ are each independently selected from H, halo, CN, NO₂, C₁₋₆ alkyl, C₂₋₆ alkenyl, OC₁₋₆ alkyl, and C₁₋₆ haloalkyl.

2. The compound of claim 1, wherein the compound of Formula I is a compound of Formula Ia: 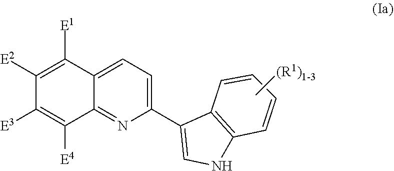
or a pharmaceutically acceptable salt thereof.

3. The compound of claim 1 or 2, wherein each R¹ is independently halo or C₁₋₆ alkyl.

4. The compound of any one of claims 1-3, wherein the compound of Formula I is a compound of Formula Ib: 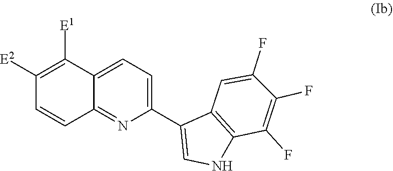
or a pharmaceutically acceptable salt thereof.

5. The compound of any one of claims 1-4, wherein E² is H or halo.

6. The compound of any one of claims 1-5, wherein the compound of Formula I is 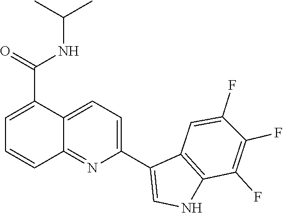 [SMILES: CC(C)NC(=O)c1cccc2nc(-c3c[nH]c4c(F)c(F)c(F)cc34)ccc12]
or a pharmaceutically acceptable salt thereof.

7. A compound of Formula II: 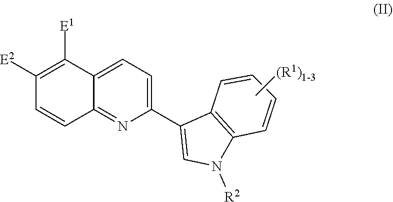
or a pharmaceutically acceptable salt thereof;
wherein
E¹ is selected from C(O)NH(C₁₋₆ alkyl), C(O)N(C₁₋₆ alkyl)₂, C(O)NH(C₃₋₆ cycloalkyl), halo, OH, CO₂H, and 5-membered heteroaryl, wherein alkyl and heteroaryl are each optionally substituted with 1, 2, or 3 substituents selected from halo, hydroxy, C₁₋₆ alkyl, C₁₋₆ haloalkyl, C₁₋₆ alkenyl, NO₂, CN, and C₃₋₆ cycloalkyl; and
E² is selected from H, halo, CN, NO₂, C₁₋₆ alkyl, C₂₋₆ alkenyl, OC₁₋₆ alkyl, and C₁₋₆ haloalkyl;
each R¹ is independently selected from the group consisting of halo, C₁₋₆ alkyl, C₁₋₆ haloalkyl, and OC₁₋₆ alkyl; and
R² is C₁₋₆ alkyl optionally substituted with OPO₃H₂.

8. The compound of claim 7, wherein the compound of Formula II is a compound of Formula IIa: 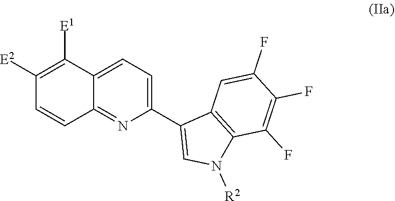
or a pharmaceutically acceptable salt thereof.

9. The compound of claim 7 or 8, wherein E¹ is C(O)NH(C₁₋₆ alkyl).

10. The compound of any one of claims 7-9, wherein E² is H or halo.

11. The compound of any one of claims 7-10, wherein the compound of Formula II is 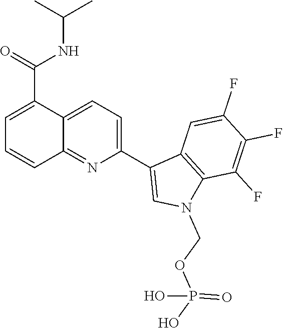 [SMILES: CC(C)NC(=O)c1cccc2nc(-c3cn(COP(=O)(O)O)c4c(F)c(F)c(F)cc34)ccc12]
or a pharmaceutically acceptable salt thereof.

12. A compound of Formula III: 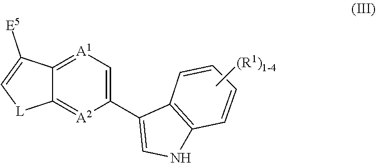
or a pharmaceutically acceptable salt thereof;
wherein
each R¹ is independently selected from the group consisting of halo, C₁₋₆ alkyl, C₁₋₆ haloalkyl, and OC₁₋₆ alkyl;
A¹ is selected from C—OH, C(OCH₃), N, CH, CF, and CCF₃;
A² is selected from N and CH;
L is selected from NH, O, and S; and
E⁵ is C(O)NH(C₁₋₆ alkyl).

13. The compound of claim 12, wherein the compound of Formula III is a compound of Formula IIIa: 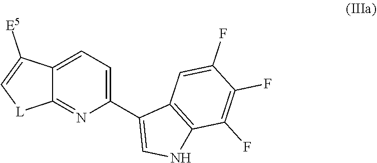
or a pharmaceutically acceptable salt thereof.

14. A pharmaceutical composition comprising a compound of any one of claims 1-13, or a pharmaceutically acceptable salt thereof, and a pharmaceutically acceptable carrier.

15. A method of treating Spinal Bulbar Muscular Atrophy (SBMA) in a subject in need thereof comprising administering to the subject a therapeutically effective amount of a compound of any one of claims 1-13, or a pharmaceutically acceptable salt thereof, or the pharmaceutical composition of claim 14.

16. A method of treating prostate cancer in a subject in need thereof comprising administering to the subject a therapeutically effective amount of a compound of any one of claims 1-13, or a pharmaceutically acceptable salt thereof, or the pharmaceutical composition of claim 14.

17. The method of claim 16, wherein the prostate cancer is castration-resistant prostate cancer (CRPC).

18. A method of modulating androgen receptor (AR) activity in a subject in need thereof comprising administering to the subject a therapeutically effective amount of a compound of any one of claims 1-13, or a pharmaceutically acceptable salt thereof, or the pharmaceutical composition of claim 14.
